SEQUENCE LISTING (66 sequences)

SEQ ID NO: 1 - protein, 18 aa, synthetic construct
MKWVTFISLLFLFSSAYS

SEQ ID NO: 2 - protein, 27 aa, synthetic construct
MDPPRPALLALLALPALLLLLLAGARA

SEQ ID NO: 3 - protein, 17 aa, synthetic construct
MGVKVLFALICIAVAEA

SEQ ID NO: 4 - protein, 20 aa, synthetic construct
MVSQALRLLCLLLGLQGCLA

SEQ ID NO: 5 - DNA, 72 bp, synthetic construct
  source 1..72: mol_type = other DNA
atgaagtgggtaacctttatttcccttctttttctctttagctcggcttattccaggggtgtgtttcgtcga

SEQ ID NO: 6 - DNA, 81 bp, synthetic construct
  source 1..81: mol_type = other DNA
atggacccgccgaggcccgcgctgctggcgctgctggcgctgcctgcgctgctgctgctgctgctggcgggcgccagggcc

SEQ ID NO: 7 - DNA, 51 bp, synthetic construct
  source 1..51: mol_type = other DNA
atgggcgtgaaagtgctgtttgcgctgatttgcattgcggtggcggaagcg

SEQ ID NO: 8 - DNA, 60 bp, synthetic construct
  source 1..60: mol_type = other DNA
atggtctcccaggccctcaggctcctctgccttctgcttgggcttcagggctgcctggct

SEQ ID NO: 9 - protein, 15 aa, synthetic construct
MLLLLLLLLLAAVAA

SEQ ID NO: 10 - protein, 16 aa, synthetic construct
MKVLLLLLFLLLLTLA

SEQ ID NO: 11 - protein, 17 aa, synthetic construct
MWLLLALLLLLAGAAAA

SEQ ID NO: 12 - protein, 18 aa, synthetic construct
MKLLLLLLLLLLLSGALA

SEQ ID NO: 13 - protein, 19 aa, synthetic construct
MRLLLLLLLLLLLLASALA

SEQ ID NO: 14 - protein, 20 aa, synthetic construct
MALLLLLLLLLLLLLALALA

SEQ ID NO: 15 - protein, 21 aa, synthetic construct
MALLALLLLLLLLLLLALAQA

SEQ ID NO: 16 - protein, 22 aa, synthetic construct
MALLRLLLLLLLLLLLLAGVLG

SEQ ID NO: 17 - protein, 23 aa, synthetic construct
MALSFALLLLLLLLLLLSSCSLA

SEQ ID NO: 18 - protein, 24 aa, synthetic construct
MARLALLLLLLLLLLLLLLPAVEA

SEQ ID NO: 19 - protein, 25 aa, synthetic construct
MGSLGLLLLLLLLLLLLLLLGGALA

SEQ ID NO: 20 - protein, 26 aa, synthetic construct
MAAPLRSALLLLALLLLLLLLGEASA

SEQ ID NO: 21 - protein, 27 aa, synthetic construct
MALARLLLLLLLLLLLLLLLLLGGVLA

SEQ ID NO: 22 - protein, 28 aa, synthetic construct
MAPGRTPLLALLLLLLLLLLLLPAAALA

SEQ ID NO: 23 - protein, 29 aa, synthetic construct
MPARGLRALLLLLLLLLLLLLLPLRPAQA

SEQ ID NO: 24 - protein, 30 aa, synthetic construct
MAAARSRGARWLKLLLLLLLLLLLPPGALA

SEQ ID NO: 25 - protein, 31 aa, synthetic construct
MALASRGSAALALLALLLLLLLALLLAGAAA

SEQ ID NO: 26 - protein, 32 aa, synthetic construct
MRAGPALAVRAALRALLLLLLLGLLLLDGSDA

SEQ ID NO: 27 - protein, 33 aa, synthetic construct
MAALAPKVSPAAAGVLLLLLLLLLLLLPGGAAA

SEQ ID NO: 28 - protein, 34 aa, synthetic construct
MARATLSAAPSNPRLLRVALLLLLLLAALRRAAG

SEQ ID NO: 29 - protein, 35 aa, synthetic construct
MAGWRRMAKALPAPAGGPACALLLLLLLLAAGAQA

SEQ ID NO: 30 - DNA, 45 bp, synthetic construct
  source 1..45: mol_type = other DNA
atgctgctgctgctgctgctgctgctgctggcggcggtggcggcg

SEQ ID NO: 31 - DNA, 48 bp, synthetic construct
  source 1..48: mol_type = other DNA
atgaaagtgctgctgctgctgctgtttctgctgctgctgaccctggcg

SEQ ID NO: 32 - DNA, 51 bp, synthetic construct
  source 1..51: mol_type = other DNA
atgtggctgctgctggcgctgctgctgctgctggcgggcgcggcggcggcg

SEQ ID NO: 33 - DNA, 54 bp, synthetic construct
  source 1..54: mol_type = other DNA
atgaaactgctgctgctgctgctgctgctgctgctgctgagcggcgcgctggcg

SEQ ID NO: 34 - DNA, 57 bp, synthetic construct
  source 1..57: mol_type = other DNA
atgcgcctgctgctgctgctgctgctgctgctgctgctgctggcgagcgcgctggcg

SEQ ID NO: 35 - DNA, 60 bp, synthetic construct
  source 1..60: mol_type = other DNA
atggcgctgctgctgctgctgctgctgctgctgctgctgctgctggcgctggcgctggcg

SEQ ID NO: 36 - DNA, 63 bp, synthetic construct
  source 1..63: mol_type = other DNA
atggcgctgctggcgctgctgctgctgctgctgctgctgctgctgctggcgctggcgcaggcg

SEQ ID NO: 37 - DNA, 66 bp, synthetic construct
  source 1..66: mol_type = other DNA
atggcgctgctgcgcctgctgctgctgctgctgctgctgctgctgctgctggcgggcgtgctgggc

SEQ ID NO: 38 - DNA, 69 bp, synthetic construct
  source 1..69: mol_type = other DNA
atggcgctgagctttgcgctgctgctgctgctgctgctgctgctgctgctgagcagctgcagcctggcg

SEQ ID NO: 39 - DNA, 72 bp, synthetic construct
  source 1..72: mol_type = other DNA
atggcgcgcctggcgctgctgctgctgctgctgctgctgctgctgctgctgctgctgccggcggtggaagcg

SEQ ID NO: 40 - DNA, 75 bp, synthetic construct
  source 1..75: mol_type = other DNA
atgggcagcctgggcctgctgctgctgctgctgctgctgctgctgctgctgctgctgctgggcggcgcgctggcg

SEQ ID NO: 41 - DNA, 78 bp, synthetic construct
  source 1..78: mol_type = other DNA
atggcggcgccgctgcgcagcgcgctgctgctgctggcgctgctgctgctgctgctgctgctgggcgaagcgagcgcg

SEQ ID NO: 42 - DNA, 81 bp, synthetic construct
  source 1..81: mol_type = other DNA
atggcgctggcgcgcctgctgctgctgctgctgctgctgctgctgctgctgctgctgctgctgctgggcggcgtgctggcg

SEQ ID NO: 43 - DNA, 84 bp, synthetic construct
  source 1..84: mol_type = other DNA
atggcgccgggccgcaccccgctgctggcgctgctgctgctgctgctgctgctgctgctgctgctgccggcggcggcgctggcg

SEQ ID NO: 44 - DNA, 87 bp, synthetic construct
  source 1..87: mol_type = other DNA
atgccggcgcgcggcctgcgcgcgctgctgctgctgctgctgctgctgctgctgctgctgctgctgccgctgcgcccggcgcaggcg

SEQ ID NO: 45 - DNA, 90 bp, synthetic construct
  source 1..90: mol_type = other DNA
atggcggcggcgcgcagccgcggcgcgcgctggctgaaactgctgctgctgctgctgctgctgctgctgctgccgccgggcgcgctggcg

SEQ ID NO: 46 - DNA, 93 bp, synthetic construct
  source 1..93: mol_type = other DNA
atggcgctggcgagccgcggcagcgcggcgctggcgctgctggcgctgctgctgctgctgctgctggcgctgctgctggcgggcgcggcggcg

SEQ ID NO: 47 - DNA, 96 bp, synthetic construct
  source 1..96: mol_type = other DNA
atgcgcgcgggcccggcgctggcggtgcgcgcggcgctgcgcgcgctgctgctgctgctgctgctgggcctgctgctgctggatggcagcgatgcg

SEQ ID NO: 48 - DNA, 99 bp, synthetic construct
  source 1..99: mol_type = other DNA
atggcggcgctggcgccgaaagtgagcccggcggcggcgggcgtgctgctgctgctgctgctgctgctgctgctgctgctgccgggcggcgcggcggcg

SEQ ID NO: 49 - DNA, 102 bp, synthetic construct
  source 1..102: mol_type = other DNA
atggcgcgcgcgaccctgagcgcggcgccgagcaacccgcgcctgctgcgcgtggcgctgctgctgctgctgctgctggcggcgctgcgccgcgcggcgggc

SEQ ID NO: 50 - DNA, 105 bp, synthetic construct
  source 1..105: mol_type = other DNA
atggcgggctggcgccgcatggcgaaagcgctgccggcgccggcgggcggcccggcgtgcgcgctgctgctgctgctgctgctgctggcggcgggcgcgcaggcg

SEQ ID NO: 51 - DNA, 711 bp, synthetic construct
  source 1..711: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaattgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagtgtcgtaccatgggctgggaggcctccactgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcgacatcaagttggacatcctttcccacaacgaggactacaccatcgtggaacagtacgaacgcgccgagggccgccactccaccggcggcatggacgagctgtacaagtaa

SEQ ID NO: 52 - DNA, 1404 bp, synthetic construct
  source 1..1404: mol_type = other DNA
ttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 53 - DNA, 1458 bp, synthetic construct
  source 1..1458: mol_type = other DNA
gaggtccagttggtagagtctgggggcggcctcgtgcagcccggtggaagccttcgcctgagttgtgcagccagtggcttcaccttctctgatagctggattcattgggtaaggcaggcacccggtaaaggtctcgagtgggttgcatggatttccccttacggggggagcacttactatgctgattccgttaaaggccgctttacaatatctgcagatacctctaaaaacactgcatacttgcaaatgaactcactgcgagctgaagacactgccgtatattactgcgcaagaaggcactggcccggtggctttgattattggggacaaggaacccttgtaactgtatctggcgggggaggaagcggtggaggtgggtcaggcggtggaggtagcggagggggtggctctgatatacagatgacacagtcacctagtagcctgtctgctagtgttggggaccgagtcactataacatgtagagcttctcaggatgtctcaaccgcagtagcctggtaccagcaaaaacctggtaaggcccccaaactcctcatatactctgcttcatttctctattctggtgtgccaagcagattcagtggtagcggcagtggcaccgacttcaccctgaccatatcttccttgcagcctgaggactttgcaacatactactgtcagcagtacctctatcatcctgcaacattcggacaaggtaccaaagtagagatcaaagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaaaagcctctccctgtccccgggtaaaggcggctctcaccaccaccatcaccactga

SEQ ID NO: 54 - DNA, 501 bp, synthetic construct
  source 1..501: mol_type = other DNA
gcccccccccggctgatctgcgacagccgggtgctggagcggtacctgctggaggccaaggaggccgagaacatcaccaccggctgcgccgagcactgcagcctgaacgagaacatcaccgtgcccgacaccaaggtgaacttctacgcctggaagcggatggaggtgggccagcaggccgtggaggtgtggcagggcctggccctgctgagcgaggccgtgctgcggggccaggccctgctggtgaacagcagccagccctgggagcccctgcagctgcacgtggacaaggccgtgagcggcctgcggagcctgaccaccctgctgcgggccctgggcgcccagaaggaggccatcagcccccccgacgccgccagcgccgcccccctgcggaccatcaccgccgacaccttccggaagctgttccgggtgtacagcaacttcctgcggggcaagctgaagctgtacaccggcgaggcctgccggaccggcgaccggtga

SEQ ID NO: 55 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 56 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 485 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSGGGGSGGGGSGGGGSGGGGSDIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSHHHHHH

SEQ ID NO: 58 - protein, 166 aa, synthetic construct
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 59 - DNA, 50 bp, synthetic construct
  source 1..50: mol_type = other DNA
acatttgcttctgacacaactgtgttcactagcaacctcaaacagacacc

SEQ ID NO: 60 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 61 - DNA, 117 bp, synthetic construct
  source 1..117: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - protein, 256 aa, synthetic construct
MEDAKNIKKGPAPFYPLEDGMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 63 - protein, 254 aa, synthetic construct
MKWVTFISLLFLFSSAYSMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 64 - protein, 263 aa, synthetic construct
MDPPRPALLALLALPALLLLLLAGARAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 65 - protein, 253 aa, synthetic construct
MGVKVLFALICIAVAEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

SEQ ID NO: 66 - protein, 256 aa, synthetic construct
MVSQALRLLCLLLGLQGCLAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFNWERVMNFEDGGVVTVTQDSSLQDGEFIYKVKLRGTNFPSDGPVMQCRTMGWEASTERMYPEDGALKGEIKQRLKLKDGGHYDAEVKTTYKAKKPVQLPGAYNVDIKLDILSHNEDYTIVEQYERAEGRHSTGGMDELYK

(19) United States
(12) Patent Application Publication
(10) Pub. No.: US 2026/0337251 A1
(43) Pub. Date: Oct. 29, 2026

(54) NOVEL SECRETORY SIGNAL PEPTIDES

(71) Applicant: THE BOARD OF REGENTS OF THE UNIVERSITY OF TEXAS SYSTEM, Austin, TX (US)

(72) Inventors: Qiang Cheng, Dallas, TX (US); Daniel J. Siegwart, Dallas, TX (US); Lukas Farbiak, Dallas, TX (US)

(21) Appl. No.: 19/114,127

(22) PCT Filed: Sep. 22, 2023
(86) PCT No.: PCT/US2023/074861

§ 371 Date: Mar. 21, 2025

Related U.S. Application Data
(60) Provisional application No. 63/376,696, filed on Sep. 22, 2022.

(51) Int. Cl. C07K 14/765; A61K 38/00; A61P 17/06; C07K 14/775; C07K 16/28; C12N 9/64

(52) U.S. Cl. CPC C07K 14/765; A61P 17/06; C07K 14/775; C07K 16/2827; C12N 9/6408; A61K 38/00; C07K 2319/02; C07K 2319/30; C12Y 304/21021

(57) ABSTRACT

Provided here are novel engineered and isolated signal peptide sequences and compositions comprising these. Also provided are compositions and methods of using these signal peptides to enable secretion of heterologous polypeptides for therapeutic, diagnostic, and commercial value.

### CROSS REFERENCE TO RELATED APPLICATIONS

This application claims the benefit of the U.S. provisional application No. 63/376,696 filed Sep. 22, 2022, the disclosure of which is herein incorporated by reference in its entirety.

### SEQUENCE LISTING

This application contains a Sequence Listing that has been submitted in WIPO ST.26 .xml format via Patent Center and is hereby incorporated by reference in its entirety. The .xml copy is named “106546-757419 UTSD 3964.xml” and is 66 KB in size.

### BACKGROUND

### 1. Field

The present invention relates to use of novel secretory signal peptides for therapeutic applications.

### 2. Background

Recent scientific discoveries have highlighted the innumerable therapeutic applications of mRNA due to its modular nature and ability to provide customizable “instructions” to create functional proteins. Clinical studies continue to confirm its favorable efficacy, further underscoring its potential as a next-generation genetic medicine wherein scientists have only begun to scratch the surface of potential applications. In parallel with advances in mRNA biology, there has been substantial progress in the field of lipid nanoparticle (LNP), polymer nanoparticle, and other approaches to mediate safe and efficacious nucleic acid delivery. Most of the research focus so far has been on amplifying intracellular expression of the encoded polypeptide. However, less focus has been placed on post-translational control of encoded proteins and targeted localization and secretion of the protein within the body.

Within the cell, a variety of unique avenues and processes exist that allow for the shuttling of proteins to various organelles such as the nucleus and mitochondria, as well as the export of proteins into the extracellular space via secretion pathways. However, in order to take advantage of these transport systems, the mRNA encoding each protein also contains a metaphorical shipping label upstream from the protein sequence known as the signal peptide (SP). This label then informs the appropriate cellular machinery to either ship the protein to a specific location within the cell or package it up for secretion into the extracellular space. Design of novel signal sequences for therapeutic mRNA could greatly benefit delivery, localization, and clearance of the encoded therapeutic protein.

### SUMMARY OF THE INVENTION

In some aspects, the current disclosure encompasses an engineered signal peptide comprising an amino acid sequence of any one of SEQ. ID. NOS. 9-29 and variant or derivative thereof. In some aspects, the signal peptide is fused to a heterologous polypeptide. In some aspects the heterologous polypeptide is a therapeutic or diagnostic polypeptide non-limiting examples of which include anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

In some aspects, the heterologous polypeptide is an enzyme, nutraceutical, food additive, taste enhancer, and/or cosmetic. In some aspects, heterologous polypeptide is a reporter polypeptide non-limiting examples of which include fluorescent protein, LacZ (b-galactosidase), CAT (chloramphenicol acetyltransferase), or luciferase. In some aspects, the fluorescent protein is any one of GFP (green fluorescent protein), BFP (blue fluorescent protein), YFP (yellow fluorescent protein), RFP (red fluorescent protein), mCherry or luciferase.

In some aspects, the current disclosure also encompasses a recombinant polynucleotide sequence comprising a nucleic acid sequence encoding the signal peptide of any one of SEQ. ID. NOS. 9-29. In some aspects the nucleic acid sequence is a DNA sequence. In some aspects the nucleic acid sequence is an RNA sequence. In some aspects the recombinant polynucleotide comprises a nucleic acid sequence of any one of SEQ. ID. NOS. 30-50 or variant or derivative thereof. In some aspects, the polynucleotide sequence comprises a ribonucleic acid sequence corresponding to any one of SEQ. ID. NOS. 30-50 or variant or derivative thereof.

In some aspects the recombinant polynucleotide encodes a heterologous polypeptide in frame with the signal peptide. In some aspects the heterologous polypeptide is a therapeutic or diagnostic polypeptide non-limiting examples of which include anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

In some aspects, the heterologous polypeptide is an enzyme, nutraceutical, food additive, taste enhancer, and/or cosmetic. In some aspects, heterologous polypeptide is a reporter polypeptide non-limiting examples of which include fluorescent protein, LacZ (b-galactosidase), CAT (chloramphenicol acetyltransferase), or luciferase. In some aspects, the fluorescent protein is any one of GFP (green fluorescent protein), BFP (blue fluorescent protein), YFP (yellow fluorescent protein), RFP (red fluorescent protein), mCherry or luciferase.

In some exemplary aspects the recombinant polynucleotide encodes a heterologous polypeptide with any one of SEQ ID. NOS. 55-58 or functional fragment, derivative or variant thereof. In some aspects, the heterologous polypeptide is a anti-PD-L1 antibody, Enbrel, mCherry or hEPO or functional fragment, derivative or variant thereof.

In some aspects the current disclosure also encompasses a therapeutic composition comprising a delivery system and a polynucleotide sequence comprising a nucleic acid sequence encoding a signal peptide and a therapeutic polypeptide. In some aspects, the signal peptide comprises an amino acid sequence of any one of SEQ. ID. NOS. 9-29 or variant or derivative thereof. In some aspects the polynucleotide sequence encoding the signal peptide comprises a nucleic acid sequence corresponding to any one of SEQ. ID. NOS. 30-50 or variant or derivative thereof.

In some aspects, the delivery system is any one of a polymers, polyplexes, lipids, lipidoids, lipoplexes, liposomes, polymer nanoparticles, nanoparticles, lipid nanoparticles (LNPs), core-shell nanoparticles, solid lipid nanoparticles, metal nanoparticles, self-assembled nucleic acid nanoparticles, hyaluronidase, nanoparticle mimics, ribonucleoproteins, positively charged peptides, small molecule RNA-conjugates, aptamer-RNA chimeras, RNA-fusion protein complexes and any combination thereof. In some aspects the delivery system is a lipid nanoparticle comprising an ionizable amino lipid. In some aspects the lipid nanoparticle further comprises one or more of a phospholipid, cholesterol, or a polymer lipid. In some aspects the delivery system comprises any one of an iPhos LNPs, mDLNPs, liver SORT LNP, lung SORT LNP, or spleen SORT LNP. In some aspects the delivery system is a controlled system selected from a synthetic material depot, polymer depot, lipid depot, controlled release hydrogel depot, or controlled release polymer depot.

In some aspects, the therapeutic composition further comprises one or more pharmaceutically acceptable excipients. In some aspects, the therapeutic polypeptide in the therapeutic composition is any one of an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

In some aspects, the current disclosure also encompasses a method of diagnosis, prophylaxis or treatment comprising, administration to a subject in need thereof, an effective amount of the compositions disclosed herein.

In some aspects, the method of diagnosis, prophylaxis or treatment comprises administration of the compositions disclosed herein through one or more of a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or transdermal route. In some aspects, the administration is via a controlled system selected from an implant, synthetic material depot, polymer depot, lipid depot, controlled release hydrogel depot, or controlled release polymer depot.

In some aspects, the subject in need of diagnosis, prophylaxis or treatment is suspected of having or diagnosed with any one of an auto-immune disorder, cancer, diabetes, cardiovascular diseases, nerve disease, bacterial infection, fungal infection, viral infection, or fibrosis. In some aspects, the subject is in need of prophylaxis. In some aspects, the therapeutic polypeptide disclosed herein is systemically secreted in the subject in need thereof. In some aspects, the therapeutic polypeptide is adapted for expression in any one of the lungs, liver or spleen. In some aspects the subject is a mammal. In some aspects, the subject is a human.

In some aspects, the current disclosure also encompasses a recombinant polypeptide comprising: a signal peptide corresponding to any one of SEQ ID NOS. 1-4 or variant or derivative thereof; and a heterologous polypeptide.

In some aspects, the current disclosure also encompasses a recombinant polynucleotide comprising a nucleic acid sequence encoding a signal peptide corresponding to any one of SEQ ID NOS. 1-4 or variant or derivative thereof and a heterologous polypeptide in frame with the signal peptide. In some aspects the nucleic acid sequence can be a DNA or an RNA sequence corresponding to SEQ. ID. NOS. 5-8 or variant or derivative thereof

In some aspects, the heterologous polypeptide is a therapeutic or diagnostic polypeptide, for example an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide. In some aspects, the heterologous polypeptide is an enzyme, nutraceutical, food additive, taste enhancer, and/or cosmetic. In some aspects, the heterologous polypeptide is a reporter polypeptide selected from a fluorescent protein, LacZ (b-galactosidase), CAT (chloramphenicol acetyltransferase), or luciferase. In some aspects, the heterologous polypeptide is for example, anti-PD-L1 antibody, Enbrel, hEPO or functional fragment, derivative or variant thereof.

In some aspects, the current disclosure also encompasses a therapeutic composition comprising a delivery system and a polynucleotide sequence comprising a nucleic acid sequence encoding a signal peptide corresponding to any one of SEQ ID NOS. 1-4 or variant of derivative thereof and a therapeutic polypeptide. In some aspects, the polynucleotide sequence encoding the signal peptide comprises a nucleic acid sequence of any one of SEQ. ID. NOS. 5-8 or variant or derivative thereof. In some aspects, the polynucleotide sequence encoding the signal peptide comprises a ribonucleic acid sequence corresponding to any one of SEQ. ID. NOS. 5-8 or variant or derivative thereof. In some aspects, non-limiting examples of delivery system for the therapeutic compositions disclosed herein are polymers, polyplexes, lipids, lipidoids, lipoplexes, liposomes, polymer nanoparticles, nanoparticles, lipid nanoparticles (LNPs), core-shell nanoparticles, solid lipid nanoparticles, metal nanoparticles, self-assembled nucleic acid nanoparticles, hyaluronidase, nanoparticle mimics, ribonucleoproteins, positively charged peptides, small molecule RNA-conjugates, aptamer-RNA chimeras, RNA-fusion protein complexes and any combination thereof. In some aspects, the delivery system is a lipid nanoparticle comprising an ionizable amino lipid. In some aspects, the lipid nanoparticle further comprises one or more of a phospholipid, cholesterol, or a polymer lipid. In some aspects, the delivery system comprises any one of an iPhos LNPs, mDLNPs, liver SORT LNP, lung SORT LNP, or spleen SORT LNP. In some aspects, the delivery system is a controlled system selected from a synthetic material depot, polymer depot, lipid depot, controlled release hydrogel depot, or controlled release polymer depot.

In some aspects, the therapeutic compositions comprising a signal peptide corresponding to SEQ ID NOS. 1-4 or variant of derivative thereof further comprise one or more pharmaceutically acceptable excipients. In some aspects, the therapeutic composition comprise a therapeutic polypeptide for example an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

In some aspects, the current disclosure also encompasses methods of diagnosis, prophylaxis or treatment comprising, administration to a subject in need thereof, an effective amount of the compositions comprising a signal peptide corresponding to SEQ ID NOS. 1-4 or variant of derivative thereof. These therapeutic compositions can be administered for example through one or more of a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or transdermal route. In some aspects, the administration is via a controlled system selected from an implant, synthetic material depot, polymer depot, lipid depot, controlled release hydrogel depot, or controlled release polymer depot. In some aspects, the subject suspected of having or diagnosed with any one of an auto-immune disorder, cancer, diabetes, cardiovascular diseases, nerve disease, bacterial infection, fungal infection, viral infection, or fibrosis. In some aspects, the subject is a mammal, for example a human, in need of prophylaxis. In some aspects, the composition is systemically secreted in the subject in need thereof or is directed for organ specific expression in any one of the lungs, liver or spleen. In some aspects, the subject is suspected of or diagnosed with any one of an auto-immune disorder, cancer, diabetes, or fibrosis.

### BRIEF DESCRIPTION OF THE DRAWINGS

Aspects of the present inventive concept are illustrated by way of example in which like reference numerals indicate similar elements and in which:

FIG. 1A provides a schematic of the construct comprising mCherry with a signal peptide at the N-terminus and its use for signal peptide screening by pDNA transfection in vitro.

FIG. 1B provides fluorescent microscopy images showing time-dependent mCherry secretion in Hela cells (exposure time, 1/30 s). mCherry signal was observed clearly in the medium after two days treatment with gLuc-mCherry. PBS, phosphate buffered saline; WT, wild-type; gLuc, Gaussia luciferase.

FIG. 1C provides quantification of mCherry fluorescence in cell lysates and medium at different time points. mCherry with no signal peptide was used as a control (“WT-mCherry”). PBS, phosphate buffered saline; WT, wild-type; gLuc, Gaussia luciferase.

FIG. 1D provides fluorescent microscopy images showing mCherry secretion in Hela cells at 72 h (exposure time, 1/70 s). PBS, phosphate buffered saline; NC, Negative Control; hAlb, human Albumin; hApoB, human Apolipoprotein B; gLuc, Gaussia luciferase; hFVII, human Factor VII.

FIG. 1E provides photographs showing cell lysates and medium imaged at 72 h. PBS, phosphate buffered saline; NC, Negative Control; hAlb, human Albumin; hApoB, human Apolipoprotein B; gLuc, Gaussia luciferase; hFVII, human Factor VII.

FIG. 1F provides quantitation of mCherry fluorescence and cell lysate and medium after 72 h, data presented as mean±s.e.m. (n=5 biologically independent samples). Hela cells in 96-well plate were treated by Lipofectamine 2000/pDNA (50 ng per well), at given time-point, cells were imaged via microscope, mCherry signal was quantified by plate reader or captured by IVIS. A two-tailed unpaired t-test was used to determine the significance of the comparisons of data (*P<0.05; **P<0.01; ***P<0.001; ****P<0.0001). PBS, phosphate buffered saline NC, Negative Control; hAlb, human Albumin; hApoB, human Apolipoprotein B; gluc, Gaussia luciferase; hFVII, human Factor VII.

FIG. 1G shows a bar graph quantifying mCherry signal in cell lysates and medium at 72 h, confirming the signal peptide functions in Huh7 cells. Data presented as mean±s.e.m. (n=4 biologically independent samples). A two-tailed unpaired t-test was used to determine the significance of the comparisons of data (*P<0.05; **P<0.01; ***P<0.001; ****P<0.0001). PBS, phosphate buffered saline; NC, Negative Control; hAlb, human Albumin; hApoB, human Apolipoprotein B; gLuc, Gaussia luciferase; hFVII, human Factor VII. PBS, phosphate buffered saline; NC, Negative Control; hAlb, human Albumin; hApoB, human Apolipoprotein B; gLuc, Gaussia luciferase; hFVII, human Factor VII.

FIG. 1H shows confocal images showing a “circle-like” signal (indicated by yellow arrows) observed in Huh7 cells when signal peptide worked well (scale bar, 50 μm). PBS, phosphate buffered saline; NC, Negative Control; hAlb, human Albumin; hApoB, human Apolipoprotein B; gLuc, Gaussia luciferase; hFVII, human Factor VII.

FIG. 1I provides quantification of mCherry fluorescence in cell lysates and medium at 72 h after transfection. Data was presented as mean±s.e.m. (n=4 biologically independent samples). A two-tailed unpaired t-test was used to determine the significance (*P<0.05; **P<0.01; ***P<0.001; ****P<0.0001). PBS, phosphate buffered saline; NC, Negative Control; hAlb, human Albumin; hApoB, human Apolipoprotein B; gLuc, Gaussia luciferase; hFVII, human Factor VII.

FIG. 1J shows images of cell lysates and medium imaged by IVIS. Huh7 cells in 96-well plate were treated by Lipofectamine 2000/pDNA (50 ng per well), at 72 hours, mCherry signal was quantified by plate reader or captured by IVIS. PBS, phosphate buffered saline; NC, Negative Control; hAlb, human Albumin; hApoB, human Apolipoprotein B; gLuc, Gaussia luciferase; hFVII, human Factor VII.

FIG. 2A shows a schematic of synthesis of hFVII-mCherry mRNA via in vitro transcription (IVT), in vivo delivery encapsulated by LNPs and imaging using microscopy and plate reader.

FIG. 2B provides graph showing time-dependent (100 ng mRNA per well) and dose-dependent (at 72 h) mCherry secretion in Huh7 cells, medium and cell lysate.

FIG. 2C shows quantification of mCherry signal in different cell lines. Cells in 96-well plate were treated with mDLNPs-mRNA, at given time-points, mCherry signal was quantified by plate reader.

FIG. 2D shows fluorescence and bright field images HEK293T cells treated with hFVII-mCherry mRNA formulations. Obvious mCherry signal was observed in the medium at day 3 after hFVII-mCherry mRNA formulation treatment. HEK293T cells in 96-well plate were treated by mDLNPs-mRNA with different doses and imaged with microscope (exposure time, 1/6 s for 24 h and 1/15 s for 72 h). A close-up reproduction of the image showing HEK293T cells treated with mRNA formulation for 24 h (exposure time, 1/6 s) is shown. The reproduced image shows the clear “circle-like” mCherry distribution (indicated by yellow arrows) in hFVII-mCherry mRNA formulation group, which was similar with pDNA delivery shown in FIG. 1H.)

FIG. 2E shows effective liver-targeted Luc mRNA delivery by mDLNP. Mice were I.V. injected with Luc mRNA dose of 0.1 mg/kg and imaged at 3 h.

FIG. 2F shows successful mCherry secretion in vivo by mDLNP/hFVII-mCherry mediated liver-targeted delivery. Mice were I.V. injected with mRNA dose of 0.5 mg/kg, serum was collected from 2 h to 72 h after treatment and mCherry signal was detected by plate reader. At 55 h and 72 h, mice were sacrificed, and tissues were imaged by IVIS.

FIG. 2G shows mCherry signal in the kidney, indicating mCherry secretion in the blood.

FIG. 2H shows successful liver-, lung- and spleen-SORT LNPs mediated Luc mRNA and hFVII-mCherry delivery in vivo. SORT LNPs showed tissue-selective Luc mRNA delivery and all achieved successful mCherry secretion into the blood. For Luc assay, mice were I.V. injected with mRNA dose of 0.1 mg/kg and imaged at 3 h. For mCherry assay, mice were I.V. injected with mRNA dose of 0.5 mg/kg and imaged at 24 h.

FIG. 3A shows a schematic of the Enbrel protein production, TNF-α binding and disease treatment on dermatitis model.

FIG. 3B is a graph showing dose-dependent cytotoxicity of human source TNF-α (hTNF-α) and mouse source TNF-α (mTNF-α) in L929 cells. Cells were incubated 24 h at Actinomycin of 1 μg/ml and various concentrations of TNF-α before determining cytotoxicity.

FIG. 3C are graphs showing cell viability in the presence of TNF-α. hFVII-Enbrel mRNA pretreated L929 cells were resistant to both mouse and human TNF-α. Cells were pretreated by mDLNP-hFVII-Enbrel mRNA with 80 ng per well mRNA, after two days, cells were challenged by TNF-α with concentrations of 0-5 ng/ml and fixed Actinomycin of 1 μg/ml. After another 24 h, cell viability was detected.

FIG. 3D is a graph quantifying dose-dependent viability rescue by LNPs following pretreatment. Cells were pretreated by mDLNP-hFVII-Enbrel mRNA with mRNA doses of 0-1.25 ng/ml, after two days, cells were challenged by TNF-α with dose of 0.1 ng/ml and fixed Actinomycin of 1 μg/ml. After another 24 h, cell viability was detected.

FIG. 3E are graphs showing dose-dependent rescue with LNPs formulation. L929 cells were pretreated by mDLNP-hFVII-Enbrel mRNA with mRNA doses of 0-1.25 ng/ml, after two days, cells were challenged by TNF-α with dose of 0.02 ng/ml and fixed Actinomycin of 1 μg/ml (top left). After another 24 h, cell viability was detected. No significant cytotoxicity was observed by mRNA formulation only (top right). Dose-dependent viability rescue by medium which was from LNPs formulation treated cells. Cells were pretreated by mDLNP-hFVII-Enbrel mRNA with mRNA doses of 0-1.25 ng/ml. After two days, medium with secreted Enbrel were transferred into new L929 cells, meanwhile cells were challenged by TNF-α with dose of 0.1 ng/ml and actinomycin of 1 μg/ml (bottom left). After another 24 h, cell viability was detected (bottom right).

FIG. 3F shows a scheme of workflow for creation and treatment of imiquimod-induced psoriasis model.

FIG. 3G shows pharmacokinetic comparison between Enbrel protein and mRNA after single dosing. Mice were I.V. injected with dose of 0.5 mg/kg protein or mRNA formulation. Serum was collected at different time-point and Enbrel was detected by ELISA kit.

FIG. 3H provides images of dorsal skin of lanolin control mice or imiquimod treated mice, which were injected with mCherry mRNA or hFVII-Enbrel mRNA formulations. Images of H&E-stained sections, Ki-67 staining and Gr-1 staining of the dorsal skin of lanolin control or imiquimod treated mice.

FIG. 3I provides quantitative measurement of epidermal thickness in three groups, and the percentage of Ki-67+ cells in epidermal basal cells (per 50 cells) quantified from h. (****P<0.0001)

FIG. 4A shows the workflow of tumor immunotherapy by hFVII-anti-PDL1 mRNA. scheme of anti-PDL1 antibody production and tumor immunotherapy are shown.

FIG. 4B shows the scheme of experimental design of tumor immunotherapy by hFVII-anti-PDL1 mRNA using MC38, MC38-Luc, and B16F10-Luc xenograft mice models.

FIG. 4C provides data for pharmacokinetic study of anti-PDL1 antibody after single dosing with LNPs-mRNA formulation. Mice were I.V. injected with dose of 0.5 mg/kg mRNA formulation. Serum was collected at different time points and anti-PDL1 was detected by ELISA kit.

FIG. 4D shows PDL1 expression on membrane surface of MC-38 cells determined by flow cytometry.

FIG. 4E shows luminescence images of MC38-Luc tumors on different time points captured by IVIS.

FIG. 4F shows luminescence images of isolated MC38-Luc tumors at day 32. Also provided are tumor images at the day 32 (1/4 cleared). (*P<0.05)

FIG. 4G shows quantified luminescence signal in tumor area at different time point after tumor immunotherapy by hFVII-anti-PDL1 mRNA. (*P<0.05; **P<0.01; ***P<0.001; ****P<0.0001)

FIG. 4H shows tumor growth of MC38 model. (*P<0.05; **P<0.01; ***P<0.001; ****P<0.0001)

FIG. 4I shows mice survival data during treatments by mRNA formulations. (*P<0.05; **P<0.01; ***P<0.001; ****P<0.0001)

FIG. 4J shows luminescence images of B16F10-Luc tumors on day 3 and day 16 treated with different formulations.

FIG. 4K shows representative PDL1 expression on membrane surface of B16F10-Luc cells determined by flow cytometry.

FIG. 4L shows a graph depicting tumor growth of B16F10-Luc model. (*P<0.05; **P<0.01; ***P<0.001; ****P<0.0001)

FIG. 4M mice survival during treatments by mRNA formulations. (*P<0.05; **P<0.01; ***P<0.001; ****P<0.0001)

FIG. 4N shows that the mRNA formulation enabled better pharmacokinetics curve compared with protein. Pharmacokinetic comparison between PD1 protein and mRNA formulation after single dosing. Mice were I.V. injected with dose of 0.5 mg/kg mRNA formulation or proteins. Serum was collected at different time-point and PD1 was detected by ELISA kit.

FIG. 5 shows a schematic of the matrix generation for constructing novel signal peptide sequences.

FIG. 6 is an overview of the signal peptide structure with the mRNA sequence and generation of the SP-mRNA using in-vitro transcription.

FIG. 7 are graphs showing the serum hEPO concentration post injection. mRNAs with SP (1-21)-hEPO were encapsulated into iPhos LNPs and administered intravenously into mice at a dosage of 0.5 mg/kg. Blood was collected from mice at 6, 24, 48, and 72 h for each construct and analyzed via hEPO enzyme-linked immunosorbent assay (ELISA) to determine serum hEPO concentration mlU/mL.

The drawing figures do not limit the present inventive concept to the specific aspects disclosed and described herein. The drawings are not necessarily to scale, emphasis instead being placed on clearly illustrating principles of certain aspects of the present inventive concept.

### DETAILED DESCRIPTION

The following detailed description references the accompanying drawings that illustrate various aspects of the present inventive concept. The drawings and description are intended to describe aspects of the present inventive concept in sufficient detail to enable those skilled in the art to practice the present inventive concept. Other components can be utilized, and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is a result of intensive investigation by the inventors to identify and/or develop novel secretory signal peptides for therapeutic and non-therapeutic applications. While much work has been done on maximizing expression of therapeutic proteins after delivery of nucleic acid into a cell, post-translational control of protein localization via elucidation and exploitation of endogenous protein translocation pathways has largely been ignored. Delivery of therapeutically relevant polypeptides encoded by nucleic acids in cells, systemically or to specific organs can be greatly beneficial to the field of nucleic acid therapeutics. The current investigation used elaborate experimental and bioinformatic approaches to either identify and isolate naturally occurring or develop synthetic secretory signal peptide that can control systemic and organ specific secretion of polypeptides. This was followed by extensive experimental validation and development of therapeutic and non-therapeutic compositions using these signal peptide sequences. These naturally occurring and synthetic signal peptide can be fused to any heterologous protein of interest to control its transport and localization. The work is a gateway to the development of novel protein therapeutics that will likely impact patient outcomes for multiple different disease conditions. Additionally, the work can also be used in development of non-therapeutic applications like diagnostics, industrial production of heterologous proteins and various laboratory purposes.

### I. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to +5%, such as less than or equal to +2%, such as less than or equal to +1%, such as less than or equal to +0.5%, such as less than or equal to +0.2%, such as less than or equal to +0.1%, such as less than or equal to +0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps, or acts are in some way inherently mutually exclusive.

The terms “nucleic acid”, “nucleic acid molecule”, and “polynucleotide” are used interchangeably herein. The terms “nucleic acid encoding . . . ”, or “nucleic acid molecule encoding . . . ” should be understood as referring to the sequence of nucleotides which encoding encodes a polypeptide.

A polynucleotide described herein may comprise one or more nucleic acids each encoding a polypeptide, operably linked to (i.e., in a functional relationship with) one or more regulatory sequences, such as a promoter. Such a polynucleotide may alternatively be referred to herein as a “nucleic acid construct” or “construct”. As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

As used herein a polynucleotide sequence “corresponding” to a sequence provide encompasses DNA, RNA and cDNA sequences with reference to the sequence. For example, with respect to a sequence provided encoding a heterologous polypeptide, both the DNA and RNA sequence encoding the heterologous polypeptide are encompassed in the term. Therefore, the polynucleotide can be identical to, or complimentary RNA sequence of the reference sequence.

As used herein, “regulatory elements” refer to any sequence elements that regulate, positively or negatively, the expression of an operably linked sequence. “Regulatory elements” include, without being limiting, a promoter, an enhancer, a leader, a transcription start site (TSS), a linker, 5′ and 3′ untranslated regions (UTRs), an intron, a polyadenylation signal, and a termination region or sequence, etc., that are suitable, necessary or preferred for regulating or allowing expression of the gene or transcribable DNA sequence in a cell. Such additional regulatory element(s) can be optional and used to enhance or optimize expression of the gene or transcribable DNA sequence. A regulatory sequence can, for example, be inducible, non-inducible, constitutive, cell-cycle regulated, metabolically regulated, and the like. A regulatory sequence may be a promoter. As used herein, the term “promoter” refers to a DNA sequence that contains an RNA polymerase binding site, a transcription start site, and/or a TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present application can thus include variants of promoter sequences that are similar in composition, but not identical to, other promoter sequence(s) known or provided herein. As used herein, the term “enhancer” refers to a region of DNA sequence that operates to initiate, assist, affect, cause, and/or promote the transcription and expression of the associated transcribable DNA sequence or coding sequence, at least in certain tissue(s), developmental stage(s) and/or condition(s). In an aspect, an enhancer is a cis enhancer. In one aspect, an enhancer is a trans enhancer.

As used herein, the term “operably linked” refers to a functional linkage between a promoter or other regulatory element and an associated transcribable DNA sequence or coding sequence of a gene (or transgene), such that the promoter, etc., operates to initiate, assist, affect, cause, and/or promote the transcription and expression of the associated transcribable DNA sequence or coding sequence, at least in certain tissue(s), developmental stage(s) and/or condition(s).

The terms “polypeptide” and “protein,” as used interchangeably herein, refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation or any other manipulation or modification, such as conjugation with a labeling component. Also included within the definition are, for example, polypeptides containing one or more analogs of an amino acid (including, for example, unnatural amino acids, etc.), as well as other modifications known in the art. The terms “polypeptide” and “protein” as used herein specifically encompass antibodies.

An amino acid sequence that is “derived from” an amino acid sequence disclosed herein can refer to an amino acid sequence that differs by one or more amino acids compared to the reference amino acid sequence, for example, containing one or more amino acid insertions, deletions, or substitutions as disclosed herein. The terms “derivative,” “variant,” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO., or having at least 50%, or at least 60%, or at least 70%, or at least 80%, or at least 90% of the length (in amino acids) of the polypeptide.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having Sulphur-containing side chains is cysteine and methionine. Preferred conservative amino acids substitution groups are valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gin; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

The term “heterologous” when used in reference to a nucleic acid molecule (such as a coding sequence) or a polypeptide (such as an enzyme) refers to a nucleic acid molecule or a protein that is not natively found in the host organism or cell. “Heterologous” also includes a native coding region, or portion thereof, that is removed from the source organism and subsequently reintroduced into the source organism in a form that is different from the corresponding native gene, e.g., not in its natural location in the organism's genome. The heterologous nucleic acid molecule is deliberately introduced into the host cell. A “heterologous” nucleic acid molecule or protein may be derived from any source, e.g., eukaryotes, prokaryotes, viruses, etc. In an aspect, the heterologous nucleic acid molecule may be derived from a eukaryote (such as, for example, another yeast) or a prokaryote (such as, for example, a bacteria). The term “heterologous” as used herein also refers to an element (nucleic acid or protein) that is derived from a source other than the endogenous source. Thus, for example, a heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

As used herein, aspects of the invention are “engineered” when they have a feature or property, whether structural or chemical, that varies from a starting point, wild type or native molecule. As used herein, with respect to a signal peptide, the term “engineered” or “synthetic” means having an amino acid sequence with one or more amino acids in the sequence different from naturally occurring signal peptides. In some aspects, these signal peptides are designed using bioinformatic analysis of naturally occurring signal peptides. In some aspects, the signal peptide may be derived but not identical to a naturally occurring signal peptide. As used herein, with respect to a polypeptide, the term “engineered” or “recombinant” means having an altered amino acid sequence as a result of the application of genetic engineering techniques to nucleic acids that encode the polypeptide, and cells or organisms that express the polypeptide. Alterations include but are not restricted to insertions, deletions, or substitutions. With respect to a nucleic acid, the term “recombinant” or “engineered” means having an altered nucleic acid sequence as a result of the application of genetic engineering techniques. Alterations include but are not restricted to insertions, deletions, or substitutions. Genetic engineering techniques include, but are not limited to, PCR and DNA cloning technologies; transfection, transformation and other gene transfer technologies; homologous recombination; site-directed mutagenesis; and gene fusion.

The term “fusion protein” or “fusion polypeptide” are used interchangeably and refer to a polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more amino acids. The protein product encoded by a fusion construct is referred to as a fusion polypeptide.

The terms “host cell,” “host cell line,” “host cell culture” “genetically modified” or “engineered host” cell as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein. In some aspects, the host cell is a genetically modified cell. As defined herein in such a cell the nuclear, organellar or extrachromosomal nucleic acid may have been transformed, modified or transduced using recombinant DNA technology to comprise a heterologous nucleic acid molecule, and is used interchangeably with “engineered cell,” “transformed cell,” and “transduced cell.” Engineered cells may be “transduced cells” wherein the cells have been infected with e.g., a modified virus, for example, a retrovirus may be used but other suitable viruses may also be contemplated such as lentiviruses. Non-viral methods may also be used, such as transfections. Engineered cells may thus also be “stably transfected cells” or “transiently transfected cells”. Transfection refers to non-viral methods to transfer DNA (or RNA) to cells such that a gene is expressed. Transfection methods are widely known in the art, such as calcium phosphate transfection, PEG transfection, and liposomal or lipoplex transfection of nucleic acids. Such a transfection may be transient but may also be a stable transfection wherein cells can be selected that have the gene construct integrated in their genome. In some cases, genetic engineering systems such as CRISPR or Argonaute may be utilized to design engineered cells that express a polypeptide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems. A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “patient” or “subject” refers to a living mammalian organism, such as a human, monkey, cow, sheep, goat, dog, cat, mouse, rat, guinea pig, or transgenic species thereof. In certain aspects, the patient or subject is a primate. Non-limiting examples of human subjects are adults, juveniles, infants, and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

A “therapeutic polypeptide” is a polypeptide that may alleviate or reduce symptoms that result from an absence or defect in a protein in a cell or subject. Alternatively, a “therapeutic polypeptide” is one that otherwise confers a benefit to a subject, e.g., anti-cancer effects or improvement in transplant survivability. As used herein, the term “therapeutic polypeptide” also encompasses proteins useful as vaccines, therapeutics, and diagnostics.

### II. Compositions

In some aspects, the current disclosure encompasses compositions and methods comprising novel naturally occurring and synthetic or engineered signal peptides and their use for controlled protein transport and localization. In some aspects, the current disclosure also encompasses use of novel delivery formulations and methods for introducing the disclosed compositions into cell or a subject in need thereof. These compositions can be used in both therapeutic (treatment and diagnostics) and non-therapeutic applications for example industrial and laboratory applications to drive extracellular secretion and distribution of a protein of interest.

In some aspects, the current disclosure encompasses engineered signal peptides. These signal peptides were developed using extensive bioinformatic analysis followed by validation studies in cell cultures and in vivo. In some aspects, the signal peptide comprises an amino acid sequence at least about 60% identical to one or more of SEQ ID. NOS: 9-29. In some aspects, the signal peptide can be at least about 60% to about 75%, or about 75% to about 80%, or about 80% to about 85%, or about 85% to about 90%, or about 90% to about 95%, or about 95% to about 100% identical to any one of SEQ ID. NOS: 9-30. In some aspects, the signal peptide can be 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID. NOS: 9-29. In some aspects, the engineered signal peptide may vary in length from 5 amino acid residue to about 100 amino acid residues. In some aspects, the signal peptide can be 5, 6, 7, 8, 9, 10, 11, 12, 13, 14, 15, 6, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75 amino acids in length. In some exemplary aspects, the signal peptide is about 10-50 residues in length. In some aspects, the engineered signal peptide may comprise a stretch of leucine amino acid residues ranging from 1-50 in number. In some aspects, the stretch of leucine amino acid residues may be split into one or two or more sets of residues, separated by one or two or more amino acid residues other than leucine.

In some aspects, the signal peptide is derived from a naturally occurring signal sequence for instance a signal peptide from any one of an albumin, hAlb; apolipoprotein B, hApoB; Gaussia luciferase, gLuc or Factor VII, hFVII sequence. In some aspects, the signal peptide comprises an amino acid sequence at least about 60% identical to one or more of SEQ ID. NOS: 1-4. In some aspects, the signal peptide can be at least about 60% to about 75%, or about 75% to about 80%, or about 80% to about 85%, or about 85% to about 90%, or about 90% to about 95%, or about 95% to about 100% identical to any one of SEQ ID. NOS: 1-4. In some aspects, the signal peptide can be 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID. NOS: 1-4.

In some aspects, the current disclosure also encompasses recombinant polypeptides comprising one of more of the signal peptides provided herein and a heterologous polypeptide. The two molecules can be adjacent in the construct or separated by a linker polypeptide that contains, 1, 2, 3, or more amino acids. Linker molecules are described, for example, in Huston, J. S., et al., PNAS 85:5879-5883 (1988), Whitlow, M., et al., Protein Engineering 6:989-995 (1993), and Newton, D. L., et al., Biochemistry 35:545-25 553 (1996). In some aspects, the recombinant polypeptide may further comprise one or more of a linker sequences, degrons, degradation tags, protease cleavage sites, and/or purifications tags. In some aspects, the heterologous polypeptide can be any polypeptide of interest. In some aspects, the heterologous polypeptide may comprise an amino acid sequence identical to or a variant, derivative or a fragment of a naturally occurring protein. In some aspects, the heterologous polypeptide may comprise a synthetic amino acid sequence. In some aspects, the heterologous polypeptide may comprise an amino acid sequence identical to or a variant, derivative or a fragment of a prokaryotic protein. In some aspects, the heterologous polypeptide may comprise an amino acid sequence identical to or a variant, derivative, or a fragment of a eukaryotic protein. In some aspect the heterologous polypeptide is a variant, derivative, or a fragment of a non-human mammalian protein (e.g., mouse, rat, rabbit, dog, monkey, gibbon, chimp, ape, baboon, cow, pig, horse, sheep, cat and other species). In some aspects, the heterologous polypeptide is a variant, derivative, or a fragment of a human protein.

In some aspects, the heterologous polypeptide is a therapeutic polypeptide. In some aspect the heterologous polypeptide is a bioactive polypeptide. In some exemplary aspects, the heterologous polypeptide has one or more of an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotics, anti-pruritic, cardiovascular, chemotherapeutic, and/or hormonal activity(s).

In some aspects, the heterologous polypeptide is a protein replacement therapeutic, polypeptide therapeutic, vaccine, viral vaccine, secreted therapeutic, anti-cancer, anti-inflammatory, anti-viral, anti-microbial, hypocholestrolemic, anti-diabetic, or anti-fibrotic polypeptide.

In some aspects, the heterologous polypeptide is a negative checkpoint regulator non-limiting examples of which include: cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, and CD73.

In some aspects, the heterologous polypeptide is a tumor antigen, non-limiting examples of which include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand.

In some aspects, the heterologous polypeptide is an antibody.

In some aspects, the heterologous polypeptide is a Chimeric Antigen Receptor (CAR).

In some aspects, the heterologous polypeptide is active as a vaccine.

In some aspects, the heterologous polypeptide is a diagnostic polypeptide. In some aspects, the heterologous polypeptide is a theragnostic. In some aspects, the heterologous polypeptide is an antibody-based diagnostic. Generally, the heterologous polypeptide is labeled with a radionucleotide (such as 111In, 99Tc, 14C, 131I, 3H, 32P or 35S) and specifically binds to a tumor antigen so that the tumor can be localized using immunoscintiography. In one aspect, heterologous polypeptides or fragments thereof bind to the extracellular domains of specific cancer biomarkers. Heterologous polypeptides for diagnostic use may be labeled with probes suitable for detection by various imaging methods. Methods for detection of probes include, but are not limited to, fluorescence, light, confocal and electron microscopy; magnetic resonance imaging and spectroscopy; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides. Additionally, probes may be bi- or multi-functional and be detectable by more than one of the methods listed.

In some aspects, the heterologous polypeptide is not a therapeutic polypeptide. In some aspects, the heterologous polypeptide is a reporter polypeptide for example a fluorescent protein like GFP (green fluorescent protein), BFP (blue fluorescent protein), YFP (yellow fluorescent protein), RFP (red fluorescent protein), mCherry, LacZ (b-galactosidase), CAT (chloramphenicol acetyltransferase), luciferase. In some aspects, the heterologous polypeptide is fused to one more detectible probe for in vitro or ex vivo use.

In some aspects, the heterologous polypeptide has industrial or commercial application. In some aspects, the polypeptide may be or useful in, for example, an enzyme, nutraceutical, food additive, taste enhancer, and/or cosmetics. In some aspects, secretion of the heterologous polypeptide disclosed herein may provide benefits in production, isolation or use of commercially relevant polypeptides.

In some exemplary aspects the heterologous polypeptide comprises a sequence at least about 60% identical to SEQ ID NOS. 55-54 or variants, derivatives, or a fragment thereof. In some exemplary aspects the heterologous polypeptide is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID. NOS: 55-58 or a functional fragment thereof.

In some aspects, the current disclosure encompasses a recombinant polypeptide comprising a signal peptide sequence that is at least 60% identical to one or more of SEQ ID. NOS: 1-4 or 9-29 and a heterologous polypeptide. In some aspects, the signal peptide sequence comprises a sequence at least about 60% to about 75%, or about 75% to about 80%, or about 80% to about 85%, or about 85% to about 90%, or about 90% to about 95%, or about 95% to about 100% identical to any one of SEQ ID. NOS: 1-4, 9-29 and a heterologous polypeptide. In some aspects, the recombinant polypeptide comprises a signal peptide sequence that can be 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID. NOS: 1-4 or 9-29 and a heterologous polypeptide. In some exemplary aspects the recombinant polypeptide comprises a signal peptide sequence at least 60% identical to SEQ ID. NOS: 1-4 or 9-29 and a heterologous polypeptide sequence at least 60% identical to SEQ. ID. NOS. 55-58 or a functional fragment thereof.

In some aspects, the current disclosure also encompasses a polynucleotide comprising a nucleic acid sequence encoding a signal peptide disclosed herein. In some aspects, the polynucleotide comprises a nucleic acid sequence encoding an amino acid sequence at least about 60% identical to any one of SEQ ID. NOS: 1-4 or 9-29. In some aspects, the polynucleotide comprises a nucleic acid sequence encoding an amino acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one or more of SEQ ID. NOS: 1-4 or 9-29. In some aspects, the polynucleotide further comprises a nucleic acid sequence encoding a heterologous polypeptide as provided herein. In some aspects, the polynucleotide further comprises a nucleic acid sequence encoding a heterologous polypeptide at least about 60% identical to SEQ ID NOS. 55-58.

In some aspects, the polynucleotide sequence may comprise a nucleic acid sequence encoding a signal peptide having a nucleic acid sequence that is at least about 60% identical to or complementary to SEQ ID. NOS. 5-8 or 30-50. In some aspects, polynucleotide sequence may comprise a nucleic acid sequence encoding a signal peptide having an nucleic acid sequence that is at least about 60% to about 75%, or about 75% to about 80%, or about 80% to about 85%, or about 85% to about 90%, or about 90% to about 95%, or about 95% to about 100% identical to or complementary to any one of SEQ ID. NOS: 5-8 or 30-50. In some aspects, the polynucleotide sequence encoding the signal peptide may comprise a nucleic acid sequence at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to or corresponding to one or more of SEQ ID. NOS: 5-8 or 30-50. In some aspects, the polynucleotide may be a deoxyribonucleotide sequence (DNA). In some aspects, the polynucleotide may be a ribonucleic acid sequence (RNA).

In some aspects, the polynucleotide sequence may further comprise a nucleic acid sequence that corresponds to a sequence encoding a heterologous polypeptide in frame with the nucleic acid sequence encoding the signal peptide. In some aspects, the polynucleotide sequence may further comprise a nucleic acid sequence that corresponds to a sequence at least about 60% identical to SEQ ID. NOS: 51-54. In some aspects, the polynucleotide may be a deoxyribonucleotide sequence (DNA). In some aspects, the polynucleotide may be a ribonucleic acid sequence (RNA).

It is contemplated that the nucleic acid encoding the heterologous polypeptide can comprises a sequence based upon a naturally-occurring sequence. Allowing for the degeneracy of the genetic code, sequences that have at least about 30%, or about 40%, or about 50%, or about 60%, or about 70%, or about 80, or about 90%, or about 95%, or about 100% of nucleotides that are identical to the nucleic acid sequence of the naturally-occurring sequence. In another aspect, the nucleic acid is a complementary sequence to a naturally occurring sequence, or complementary to 75%, 80%, 85%, 90%, 95% and 100%. Polynucleotides encoding 50, 100, 250, 500, 1000, 1500, 2000, 2500, 3000 or longer and intermediate sizes are contemplated herein. In some aspects, the nucleic acid encoding the heterologous polypeptide may be derived from genomic DNA cloned directly from the genome of a particular organism. In some aspects, however, the nucleic acid would comprise complementary DNA (cDNA). In some aspects, the nucleic acid would comprise an mRNA encoding the heterologous polypeptide and an in-frame signal peptide.

In some aspects, the polynucleotide disclosed herein may further comprise one or more of, without being limiting, a promoter, an enhancer, a leader, a transcription start site (TSS), a linker, 5′ and 3′ untranslated regions (UTRs), Kozak sequence, an intron, a polyadenylation signal, cap sequences, enhancers, viral sequences, IRES sequences and a termination region or sequence, that are suitable, necessary or preferred for regulating or allowing expression of the heterologous protein in the cell. may comprise one or more regions or parts which act or function as an untranslated region. Where polynucleotides are designed to encode at least one polypeptide of interest, the polynucleotides may comprise one or more of these untranslated regions. By definition, wild type untranslated regions (UTRs) of a gene are transcribed but not translated. In mRNA, the 5′ UTR starts at the transcription start site and continues to the start codon but does not include the start codon; whereas, the 3′ UTR starts immediately following the stop codon and continues until the transcriptional termination signal. The regulatory features of a UTR can be incorporated into the polynucleotides of the present invention to, for example, enhance the stability of the molecule. The specific features can also be incorporated to ensure controlled down-regulation of the transcript in case they are misdirected to undesired organs sites. In some aspects, any suitable naturally occurring or synthetic UTR sequence can be incorporated into the polynucleotides disclosed herein. Other non-UTR sequences may also be used as regions or subregions within the polynucleotides. For example, introns or portions of introns sequences may be incorporated into regions of the polynucleotides of the invention. Incorporation of intronic sequences may increase protein production as well as polynucleotide levels. Combinations of features may be included in flanking regions and may be contained within other features. For example, the ORF may be flanked by a 5′ UTR which may contain a strong Kozak translational initiation signal and/or a 3′ UTR which may include an oligo (dT) sequence for templated addition of a poly-A tail. 5′ UTR may comprise a first polynucleotide fragment and a second polynucleotide fragment from the same and/or different genes.

In some embodiments, the 5′ UTR sequence may be SEQ ID NO: 59. In some embodiments the 3′ UTR sequence may be SEQ ID NO: 60.

In some aspects, the polynucleotides disclosed herein may be assembled inside a cell. In some aspects, the polynucleotide may be synthesized in vivo. In some aspects, the polynucleotide may be synthesized in vitro using methods known in the art for example in vitro transcription, DNA, RNA and cDNA synthesis methods. In some aspects, the polynucleotides disclosed herein maybe incorporated into a suitable viral vector, expression cassette, expression vector, transposon, extrachromosomal element, integrated into the chromosome, host cell, delivery systems.

In some aspects, the polynucleotide is a chemically modified polynucleotide. In some aspects, the polynucleotide may comprise one or more modified nucleosides comprising a modified sugar moiety. Such compounds comprising one or more sugar-modified nucleosides may have desirable properties, such as enhanced nuclease stability relative to an oligonucleotide comprising only nucleosides comprising naturally occurring sugar moieties. In some aspects, modified sugar moieties are substituted sugar moieties. In some aspects, modified sugar moieties are sugar surrogates. Such sugar surrogates may comprise one or more substitutions corresponding to those of substituted sugar moieties. In some aspects, the modified polynucleotide may comprise a modified backbone, for example, phosphorothioates, phosphotriesters, morpholinos, methyl phosphonates, short chain alkyl or cycloalkyl intersugar linkages or short chain heteroatomic or heterocyclic inter sugar linkages.

In some aspects, modified sugar moieties are substituted sugar moieties comprising one or more non-bridging sugar substituent, including but not limited to substituents at the 2′ and/or 5′ positions. Examples of sugar substituents suitable for the 2′-position, include, but are not limited to: 2′-F, 2′-OCH₃ (“OMe” or “O-methyl”), and 2′-O(CH₂)₂OCH₃ (“MOE”). In certain aspects, sugar substituents at the 2′ position is selected from allyl, amino, azido, thio, O-allyl, O—C1-C10 alkyl, O—C1-C10 substituted alkyl; OCF₃, O(CH₂)₂SCH₃, O(CH₂)₂—O—N(Rm)(Rn), and O—CH₂—C(═O)—N(Rm)(Rn), where each Rm and Rn is, independently, H or substituted or unsubstituted C1-C10 alkyl. Examples of sugar substituents at the 5′-position, include, but are not limited to: 5′-methyl (R or S); 5′-vinyl, and 5′-methoxy. In some aspects, substituted sugars comprise more than one non-bridging sugar substituent, for example, T-F-5′-methyl sugar moieties (see, e.g., PCT International Application WO 2008/101157, for additional 5′,2′-b is substituted sugar moieties and nucleosides).

Nucleosides comprising 2′-substituted sugar moieties are referred to as 2′-substituted nucleosides. In some aspects, a 2′-substituted nucleoside comprises a 2′-substituent group selected from halo, allyl, amino, azido, SH, CN, OCN, CF₃, OCF₃, O, S, or N(Rm)-alkyl; O, S, or N(Rm)-alkenyl; O, S or N(Rm)-alkynyl; O-alkylenyl-O-alkyl, alkynyl, alkaryl, aralkyl, O-alkaryl, O-aralkyl, O(CH₂)₂SCH₃, O(CH₂)₂—O—N(Rm)(Rn) or O—CH₂—C(═O)—N(Rm)(Rn), where each Rm and Rn is, independently, H, an amino protecting group or substituted or unsubstituted C1-C10 alkyl. These 2′-substituent groups can be further substituted with one or more substituent groups independently selected from hydroxyl, amino, alkoxy, carboxy, benzyl, phenyl, nitro (NO₂), thiol, thioalkoxy (S-alkyl), halogen, alkyl, aryl, alkenyl and alkynyl.

In some aspects, a 2′-substituted nucleoside comprises a 2′-substituent group selected from F, NH₂, N₃, OCF₃, O—CH₃, O(CH₂)₃NH₂, CH₂—CH—CH₂, O—CH₂—CH—CH₂, OCH₂CH₂OCH₃, O(CH₂)₂SCH₃, O—(CH₂)₂—O—N(Rm)(Rn), O(CH₂)₂O(CH₂)₂N(CH₃)₂, and N-substituted acetamide (O—CH₂—C(═O)—N(Rm)(Rn) where each Rm and R. is, independently, H, an amino protecting group or substituted or unsubstituted C1-C10 alkyl. In some aspects, a 2′-substituted nucleoside comprises a sugar moiety comprising a 2′-substituent group selected from F, OCF₃, O—CH₃, O₂CH₂OCH₃, O(CH₂)₂SCH₃, O(CH₂)₂—O—N(CH₃)₂, —O(CH₂)₂O(CH₂)₂N(CH₃)₂, and O—CH₂—C(═O)—N(H)CH₃. In some aspects, a 2′-substituted nucleoside comprises a sugar moiety comprising a 2′-substituent group selected from F, O—CH₃, and OCH₂CH₂OCH₃.

Certain modified sugar moieties comprise a bridging sugar substituent that forms a second ring resulting in a bicyclic sugar moiety. In some such aspects, the bicyclic sugar moiety comprises a bridge between the 4′ and the 2′ furanose ring atoms. Examples of such 4′ to 2′ sugar substituents, include, but are not limited to: —[C(Ra)(Rb)]—, —[C(Ra)(Rb)]n-O—, —C(RaRb)—N(R)—O— or, —C(RaRb)—O—N(R)—; 4′-CH₂-2′, 4′-(CH₂)₂-2′, 4′-(CH₂)—O-2′ (LNA); 4′-(CH₂)—S-2′; 4′-(CH₂)₂—O-2′ (ENA); 4′-CH(CH₃)—O-2′ (cEt) and 4′-CH(CH₂OCH₃)—O-2′, and analogs thereof (see, e.g., U.S. Pat. No. 7,399,845); 4′-C(CH₃)(CH₃)—O-2′ and analogs thereof, (see, e.g., WO 2009/006478); 4′-CH₂—N(OCH₃)-2′ and analogs thereof (see, e.g., WO2008/150729); 4′-CH₂—O—N(CH₃)-2′ (see, e.g., US2004/0171570, published Sep. 2, 2004); 4′-CH₂—O—N(R)-2′, and 4′-CH₂—N(R)—O-2′-, wherein each R is, independently, H, a protecting group, or C1-C12 alkyl; 4′-CH₂—N(R)—O-2′, wherein R is H, C1-C12 alkyl, or a protecting group (see, U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ and analogs thereof (see, PCT International Application WO 2008/154401).

In some aspects, such 4′ to 2′ bridges independently comprise from 1 to 4 linked groups independently selected from —[C(Ra)(Rb)]n-, —C(Ra)—C(Rb)—, —C(Ra)═N—,—C(═NRa)—, —C(═O)—, —C(═S)—, —O—, —Si(Ra)2-S(═O)x-, and —N(Ra)—; wherein: x is 0, 1, or 2; n is 1, 2, 3, or 4; each Ra and Rb is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and each J1 and J2 is, independently, H, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl, or a protecting group.

Nucleosides comprising bicyclic sugar moieties are referred to as bicyclic nucleosides or BNAs. Bicyclic nucleosides include, but are not limited to, (A) α-L-Methyleneoxy (4′-CH2-O-2′) BNA, (B) 3-D-Methyleneoxy (4′-CH2-O-2′) BNA (also referred to as locked nucleic acid or LNA), (C) Ethyleneoxy (4′-(CH₂)2-O-2′) BNA, (D) Aminooxy (4′-CH2-O—N(R)-2′) BNA, (E) Oxyamino (4′-CH2-N(R)—O-2′) BNA, (F) Methyl(methyleneoxy) (4′-CH(CH3)-O-2′) BNA (also referred to as constrained ethyl or cEt), (G) methylene-thio (4′-CH2-S-2′) BNA, (H) methylene-amino (4′-CH2-N(R)-2′) BNA, (I) methyl carbocyclic (4′-CH2-CH(CH3)-2′) BNA, (J) propylene carbocyclic (4′-(CH2)3-2′) BNA, and (K) Methoxy (ethyleneoxy) (4′-CH(CH2OMe)-O-2′) BNA (also referred to as constrained MOE or cMOE).

Additional bicyclic sugar moieties are known in the art, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 129 (26) 8362-8379 (Jul. 4, 2007); Elayadi et al., Curr. Opinion Invens. Drugs, 2001, 2, 5561; Braasch et al., Chem. Biol., 2001, 8, 1-7; Orum et al., Curr. Opinion Mol. Ther., 2001, 3, 239-243; U.S. Pat. Nos. 7,053,207, 6,268,490, 6,770,748, 6,794,499, 7,034,133, 6,525,191, 6,670,461, and 7,399,845; WO 2004/106356, WO 1994/14226, WO 2005/021570, and WO 2007/134181; U.S. Patent Publication Nos. US 2004/0171570, US 2007/0287831, and US 2008/0039618; U.S. Ser. Nos. 12/129,154, 60/989,574, 61/026,995, 61/026,998, 61/056,564, 61/086,231, 61/097,787, and 61/099,844; and PCT International Applications Nos. PCT/US2008/064591, PCT/US2008/066154, and PCT/US2008/068922.

In some aspects, bicyclic sugar moieties and nucleosides incorporating such bicyclic sugar moieties are further defined by isomeric configuration. For example, a nucleoside comprising a 4′-2′ methylene-oxy bridge, may be in the .alpha.-L configuration or in the .beta.-D configuration. Previously, α-L-methyleneoxy (4′-CH2-O-2′) bicyclic nucleosides have been incorporated into antisense polynucleotides that showed antisense activity (Frieden et al., Nucleic

In some aspects, substituted sugar moieties comprise one or more non-bridging sugar substituent and one or more bridging sugar substituent (e.g., 5′-substituted and 4′-2′ bridged sugars; PCT International Application WO 2007/134181, wherein LNA is substituted with, for example, a 5′-methyl or a 5′-vinyl group).

In some aspects, modified sugar moieties are sugar surrogates. In some such aspects, the oxygen atom of the naturally occurring sugar is substituted, e.g., with a sulfur, carbon or nitrogen atom. In some such aspects, such modified sugar moiety also comprises bridging and/or non-bridging substituents as described above. For example, certain sugar surrogates comprise a 4′-sulfur atom and a substitution at the 2′-position (see, e.g., published U.S. Patent Application US 2005/0130923) and/or the 5′ position. By way of additional example, carbocyclic bicyclic nucleosides having a 4′-2′ bridge have been described (see, e.g., Freier et al., Nucleic Acids Research, 1997, 25 (22), 4429-4443 and Albaek et al., J. Org. Chem., 2006, 71, 7731-7740).

In some aspects, sugar surrogates comprise rings having other than 5-atoms. For example, in some aspects, a sugar surrogate comprises a six-membered tetrahydropyran (THP). Such tetrahydropyrans may be further modified or substituted. Nucleosides comprising such modified tetrahydropyrans include, but are not limited to, hexitol nucleic acid (HNA), anitol nucleic acid (ANA), manitol nucleic acid (MNA) (see Leumann, C J. Bioorg. & Med. Chem. (2002) 10:841-854), and fluoro HNA (F-HNA).

Many other bicyclo and tricyclo sugar surrogate ring systems are also known in the art that can be used to modify nucleosides for incorporation into antisense compounds (see, e.g., review article: Leumann, J. C, Bioorganic & Medicinal Chemistry, 2002, 10, 841-854).

Combinations of modifications are also provided without limitation, such as 2′-F-5′-methyl substituted nucleosides (see PCT International Application WO 2008/101157 for other disclosed 5′,2′-b is substituted nucleosides) and replacement of the ribosyl ring oxygen atom with S and further substitution at the 2′-position (see U.S. Patent Publication US 2005/0130923) or alternatively 5′-substitution of a bicyclic nucleic acid (see PCT International Application WO 2007/134181 wherein a 4′-CH2-O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., 2007).

In some aspects, the present invention provides polynucleotides comprising modified nucleosides. Those modified nucleotides may include modified sugars, modified nucleobases, and/or modified linkages. The specific modifications are selected such that the resulting polynucleotides possess desirable characteristics. In some aspects, polynucleotides comprise one or more RNA-like nucleosides. In some aspects, polynucleotides comprise one or more DNA-like nucleotides.

In some aspects, nucleosides of the present invention comprise one or more unmodified nucleobases. In certain aspects, nucleosides of the present invention comprise one or more modified nucleobases.

In some aspects, modified nucleobases are selected from: universal bases, hydrophobic bases, promiscuous bases, size-expanded bases, and fluorinated bases as defined herein. 5-substituted pyrimidines, 6-azapyrimidines and N-2, N-6 and 0-6 substituted purines, including 2-aminopropyladenine, 5-propynyluracil; 5-propynylcytosine; 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, 3-deazaguanine and 3-deazaadenine, universal bases, hydrophobic bases, promiscuous bases, size-expanded bases, and fluorinated bases as defined herein. Further modified nucleobases include tricyclic pyrimidines such as phenoxazine cytidine([5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g., 9-(2-aminoethoxy)-H-pyrimido[5,4-13][1,4]benzoxazin-2(3H)-one), carbazole cytidine (2H-pyrimido[4,5-b]indol-2-one), pyridoindole cytidine (H-pyrido[3′,2′: 4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; those disclosed by Englisch et al., 1991; and those disclosed by Sanghvi, Y. S., 1993.

Representative United States Patents that teach the preparation of certain of the above noted modified nucleobases as well as other modified nucleobases include without limitation, U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594, 121; 5,596,091; 5,614,617; 5,645,985; 5,681,941; 5,750,692; 5,763,588; 5,830,653 and 6,005,096, each of which is herein incorporated by reference in its entirety.

In some aspects, the present invention provides polynucleotides comprising linked nucleosides. In such aspects, nucleosides may be linked together using any internucleoside linkage. The two main classes of internucleoside linking groups are defined by the presence or absence of a phosphorus atom. Representative phosphorus containing internucleoside linkages include, but are not limited to, phosphodiesters (P═O), phosphotriesters, methylphosphonates, phosphoramidate, and phosphorothioates (P—S). Representative non-phosphorus containing internucleoside linking groups include, but are not limited to, methylenemethylimino (—CH2-N(CH3)-O—CH2-), thiodiester (—O—C(O)—S—), thionocarbamate (—O—C(O)(NH)—S—); siloxane (—O—Si(H)2-O—); and N,N′-dimethylhydrazine (—CH2-N(CH3)-N(CH3)-). Modified linkages, compared to natural phosphodiester linkages, can be used to alter, typically increase, nuclease resistance of the polynucleotide. In some aspects, internucleoside linkages having a chiral atom can be prepared as a racemic mixture, or as separate enantiomers. Representative chiral linkages include, but are not limited to, alkylphosphonates and phosphorothioates. Methods of preparation of phosphorous-containing and non-phosphorous-containing internucleoside linkages are well known to those skilled in the art.

The polynucleotides described herein may comprise one or more asymmetric centers and thus give rise to enantiomers, diastereomers, and other stereoisomeric configurations that may be defined, in terms of absolute stereochemistry, as (R) or(S), a or R such as for sugar anomers, or as (D) or (L) such as for amino acids etc. Included in the antisense compounds provided herein are all such possible isomers, as well as their racemic and optically pure forms.

Neutral internucleoside linkages include without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), and thioformacetal (3′-5-CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

Additional modifications may also be made at other positions on the polynucleotide, particularly the 3′ position of the sugar on the 3′ terminal nucleotide and the 5′ position of 5′ terminal nucleotide. For example, one additional modification of the polynucleotides of the present invention involves chemically linking to the polynucleotide one or more additional moieties or conjugates which enhance the activity, cellular distribution or cellular uptake of the polynucleotide. Such moieties include but are not limited to lipid moieties such as a cholesterol moiety (Letsinger et al., 1989), cholic acid (Manoharan et al., 1994), a thioether, e.g., hexyl-5-tritylthiol (Manoharan et al., 1992; Manoharan et al., 1993), a thiocholesterol (Oberhauser et al., 1992), an aliphatic chain, e.g., dodecandiol or undecyl residues (Saison-Behmoaras et al., 1991; Kabanov et al., 1990; Svinarchuk et al., 1993), a phospholipid, e.g., di-hexadecyl-rac-glycerol or triethylammonium 1,2-di-O-hexadecyl-rac-glycero-3-H-phosphonate (Manoharan et al., 1995; Shea et al., 1990), a polyamine or a polyethylene glycol chain (Manoharan et al., 1995), or adamantane acetic acid (Manoharan et al., 1995), a palmityl moiety (Mishra et al., 1995), or an octadecylamine or hexylamino-carbonyl-oxycholesterol moiety (Crooke et al., 1996).

Representative United States patents that teach the preparation of such polynucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574, 142; 5,585,481; 5,587,371; 5,595,726; 5,597,696; 5,599,923; 5,599,928 and 5,688,941, each of which is herein incorporated by reference.

In some aspects, the current disclosure also encompasses a host cell comprising the polynucleotide disclosed herein. In some aspects, the host cell or a population of host cell comprises a polynucleotide comprising a nucleic acid sequence encoding a signal peptide. In some aspects, the host cell or a population of host cell comprises a polynucleotide comprising a nucleic acid sequence encoding a heterologous polypeptide in frame with the signal peptide. In some aspects, the host cell is a eukaryotic cell. In some aspects, the host cell is a mammalian cell. In some exemplary aspects, the host cell is a human cell. In some aspects, the host cell is an in vitro cell line or an ex vivo cell. In some aspects, the host cell is present in vivo. In some aspects, the host cell is a somatic cell. In some aspects, the host cell is a differentiated cell. In some aspects, the host cell is a stem cell. In some aspects, the host cell is a tumor cell. In some aspects, the host cell is selected form the group comprising: CHO-K1 cells; HEK293 cells; Hela cells; Caco2 cells; U2-OS cells; NIH 3T3 cells; NSO cells; SP2 cells; CHO—S cells; DG44 cells; K-562 cells, U-937 cells; MRC5 cells; IMR90 cells; Jurkat cells; HepG2 cells; Hela cells; HT-1080 cells; HCT116 cells; Hu-h7 cells; Huvec cells; Molt 4 cells. In some exemplary aspects, the host cell is selected from a Hela A549 cell, Huh7 cell or IGROV1.

Compositions for Cell Cultures, Tissue Culture, Ex Vivo and/or In Vivo Delivery

In some aspects, the current disclosure encompasses compositions comprising the polypeptides or polynucleotides provided herein combined with a suitable delivery system for cell culture, tissue culture, ex vivo and/or in vivo delivery. In some aspects, the suitable delivery system is one that introduces the polypeptides or polynucleotides disclosed herein to a cell.

In some aspects, the current disclosure encompasses compositions comprising the polynucleotides comprising a nucleic acid sequence encoding a signal peptide and a recombinant polypeptide combined with a suitable delivery system. In some aspects, the current disclosure encompasses the use of any suitable delivery system known in the art.

In some aspects, the suitable delivery system may be a viral vector. In some aspects, the viral vector is an RNA viral vector. In some aspects, the viral vector is a DNA viral vector. Non-limiting examples of suitable viral vectors include adenovirus, adeno associated virus (AAV), retrovirus, herpesvirus, lentivirus, poxvirus, or papilloma virus vector.

In some aspects, the delivery system is a non-viral delivery system. Non-limiting examples of non-viral delivery systems include polymers, polyplexes, lipids, lipidoids, lipoplexes, liposomes, lipid fusion constructs, polymer nanoparticles, nanoparticles, lipid nanoparticles (LNPs), core-shell nanoparticles, solid lipid nanoparticles, metal nanoparticles, self-assembled nucleic acid nanoparticles, hyaluronidase, nanoparticle mimics, ribonucleoproteins, positively charged peptides, small molecule RNA-conjugates, aptamer-RNA chimeras, RNA-fusion protein complexes and any combination thereof.

In some aspects, the polynucleotide of the current disclosure can be formulated using natural and/or synthetic polymers. The polymer may comprise one or more of polymer such as, but not limited to, polyethenes, polyethylene glycol (PEG), poly(I lysine) (PLL), PEG grafted to PLL, cationic lipopolymer, biodegradable cationic lipopolymer, polyethyleneimine (PEI), cross-linked branched poly (alkylene imines), a polyamine derivative, a modified poloxamer, a biodegradable polymer, biodegradable block copolymer, biodegradable random copolymer, biodegradable polyester copolymer, biodegradable polyester block copolymer, biodegradable polyester block random copolymer, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly (ethylene imine), poly (serine ester), poly (L-lactide-co-L-lysine), poly(4-hydroxy-L-proline ester), acrylic polymers, methyl methacrylate copolymers, ethoxyethyl methacrylates, cyanoethyl methacrylate, amino alkyl methacrylate copolymer, poly(acrylic acid), poly(methacrylic acid), polycyanoacrylates, amine-containing polymers or combinations thereof. Non-limiting examples of polymers which may be used for delivery include, but are not limited to, Dynamic POLYCONJUGATE™ formulations from MIRUS® Bio (Madison, Wis.) and Roche Madison (Madison, Wis.), PHASERX™ polymer formulations such as, without limitation, SMARTT POLYMER TECHNOLOGY™ (Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly(lactic-co-glycolic acid) (PLGA) polymers. RONDEL™ (RNAi/Oligonucleotide Nanoparticle Delivery) polymers (Arrowhead Research Corporation, Pasadena, Calif.) and pH responsive co-block polymers such as, but not limited to, PHASERX™ (Seattle, Wash.).

In some aspects, the delivery system comprises a liposome. Non-limiting examples include but are not restricted to N-[1-(2,3-dioleoloxy)-propyl]-N, N, N-trimethylammonium chloride (DOTMA), N-[1-(2,3-dioleoloxy)-propyl]-N, N,N-trimethylammonium methyl sulfate (DOTAP), dioleoyl dimethylammonium-propane (DODAP) and dipalmitoylphosphatidyl ethanolamine (DOPE) or dioleoyl phosphatidylethanolamine (DPPE), distearoyl phosphatidylcholine (DSPC), DPPC (1,2-dipalmitoyl-sn-glycero-3-phosphocholine), DMPE (1,2-dimyristoyl-sn-glycero-3-phosphoethanolamine), DOPG (,2-dioleoyl-sn-glycero-3-phospho-(1′-rac-glycerol)), 3β-[N—(N′,N′-dimethylaminoethane) carbamoyl]cholesterol (DC-Chol), 2,3,-dioleyloxy-N-[2 (sperminecarboxamido)ethyl]-N, N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB) and any combination thereof.

In some aspects, the delivery system comprises one or more nanoparticles. Nanoparticles can be solid in nature, and comprise materials including polysaccharides, lipids, proteins, polymers, biodegradable polymers, metal oxides, and any combination thereof. Other nanoparticles are in a liquid form, and are mainly liposomes, micelles or emulsion systems composed of amphiphilic molecules or polymers. Lipid nanoparticles (LNP) are one of the most promising types of nanoparticles due to high encapsulating efficiency of nucleic acids, high stability and compatibility with biologic environments.

In some aspects, the LNPs may be made from cationic, anionic, zwitterionic or neutral lipids or any combination thereof. LNPs may also be comprised of hydrophobic lipids, hydrophilic lipids, or both hydrophobic and hydrophilic lipids. Any lipid or combination of lipids that are known in the art can be used to produce an LNP. Non-limiting examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Non-limiting examples of often used cationic lipids are: polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin, Eufectins, 98N12-5, C12-200, DDAB, DLin-KC2-DMA (KC2), DLin-MC3-DMA (MC3), XTC, MD1, and 7C1. Non-limiting examples of often used neutral lipids are: DPSC, DPPC, POPC, DOPE, and SM. Non-limiting examples of often used PEG-modified lipids are: PEG-DMG, PEG-DSG, PEG-CerC14, and PEG-CerC20. Neutral lipids, such as the fusogenic phospholipid DOPE or the membrane component cholesterol, may be included in LNPs to enhance transfection activity and nanoparticle stability. In some aspects, the lipid nanoparticles comprise an ionizable amino lipid (e.g., heptatriaconta-6,9,28,31-tetraen-19-yl 4-(dimethylamino)butanoate, DLin-MC3-DMA), a phospholipid for example phosphatidylcholine (PC), phosphatidylglycerol (PG), phosphatidylethanolamine (PE), phosphatidylserine (PS), phosphatidic acid (PA), phosphatidylinositol (PI), egg phosphatidylcholine (EPC), egg phosphatidylglycerol (EPG), egg phosphatidylethanolamine (EPE), egg phosphatidylserine (EPS), egg phosphatidic acid (EPA), egg phosphatidylinositol (EPI), soy phosphatidylcholine (SPC), soy phosphatidylglycerol (SPG), soy phosphatidylethanolamine (SPE), soy phosphatidylserine (SPS), soy phosphatidic acid (SPA), soy phosphatidylinositol (SPI), dipalmitoylphosphatidylcholine (DPPC), 1,2-dioleoyl-sn-glycero-3-phosphatidylcholine (DOPC), dimyristoylphosphatidylcholine (DMPC), dipalmitoylphosphatidylglycerol (DPPG), diolelphosphatidylglycerol (DOPG), dimyristoylphosphatidylglycerol (DMPG), hexadecylphosphocholine (HEPC), hydrogenated soy phosphatidylcholine (HSPC), distearoylphosphatidylcholine (DSPC), distearoylphosphatidylglycerol (DSPG), dioleylphosphatidylethanolamine (DOPE), palmitoylstearoylphosphatidylcholine (PSPC), palmitoylstearoylphosphatidylglycerol (PSPG), monooleoylphosphatidylethanolamine (MOPE), 1-palmitoyl-2-oleoyl-sn-glycero-3-phosphatidylcholine (POPC), polyethyleneglycol distearoylphosphatidylethanolamine (PEG-DSPE), dipalmitoylphosphatidylserine (DPPS), 1,2-dioleoyl-sn-glycero-3-phosphatidylserine (DOPS), dimyristoylphosphatidylserine (DMPS), distearoylphosphatidylserine (DSPS), dipalmitoylphosphatidic acid (DPPA), 1,2-dioleoyl-sn-glycero-3-phosphatidic acid (DOPA), dimyristoylphosphatidic acid (DMPA), distearoylphosphatidic acid (DSPA), dipalmitoylphosphatidylinositol (DPPI), 1,2-dioleoyl-sn-glycero-3-phosphatidylinositol (DOPI), dimyristoylphosphatidylinositol (DMPI), distearoylphosphatidylinositol (DSPI), and a mixture thereof., a cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5 (3): 498-507. A variety of such LNP systems are known and are disclosed in, for example, but not limited to, Hou, X. et al. Lipid nanoparticles for mRNA delivery. Nat Rev Mater 6, 1078-1094 (2021), U.S. Pat. Nos. 7,166,745; 7,173,154; 7,323,594; 7,470,817; 7,479,573; 7,601,872; 7,915,450; 8,158,827; 8,785,200; 9,358,300, International Patent Publication No. WO 2016/011203, and U.S. Patent Publication No. 2017/0107539, all of which are hereby expressly incorporated by reference in their entirety as though fully set forth herein. In some aspects, the lipid nanoparticle may comprise a combination of lipids for example a cationic lipid, a phospholipid (for example and a PEGylated lipid for example an iPhos LNPs (9A1-P9/Cholesterol/DODAP/DMG-PEG, 25:30:30:1 mol/mol; 18:1 9A1-P9: nucleic acid, wt/wt). In some aspects, the lipid nanoparticle is an Organ Targeting (SORT) lipid nanoparticle as provided in U.S. Pat. No. 11,304,911B2, the entirety of which is incorporated by reference. In some aspects, the LNP may be selected from any one of iPhos LNPs, mDLNPs, liver SORT LNP, lung SORT LNP, or spleen SORT LNP and any combination thereof.

In some exemplary aspects, the delivery system comprises one or more of lipid nanoparticle (LNPs). In some aspects, a lipid nanoparticle has a mean diameter between about 10 and about 1000 nm. In some aspects, a lipid nanoparticle has a diameter that is less than 300 nm. In some aspects, a lipid nanoparticle has a diameter between about 10 and about 300 nm. In some aspects, a lipid nanoparticle has a diameter that is less than 200 nm. In some aspects, a lipid nanoparticle has a diameter between about 25 and about 200 nm. In some aspects, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

In some aspects, of the compositions comprising LNPs, the mole ratio of the LNP to the nucleic acid from about 5:1 to about 1000:1. In some aspects, the mole ratio of the LNP to the nucleic acid is from about 100:1 to about 1000:1. In other aspects, the mole ratio is from about 250:1 to about 750:1. In some aspect mole ratio is 5:1, 10:1, 50:1, 100:1, 200:1, 250:1, 300:1, 350:1, 400:1, 450:1, 500:1, 550:1, 600:1, 650:1, 700:1, 750:1, 800:1, 850:1, 900:1, 950:1, 100:1 or any intermediate ratio.

In some aspects, the compositions disclosed herein may be a therapeutic composition and may further comprise one or more pharmaceutically acceptable excipients. Pharmaceutically acceptable excipients used in the manufacture of pharmaceutical compositions include, but are not limited to, inert diluents, dispersing and/or granulating agents, suspension aids, isotonic agents, thickening agents, surface active agents and/or emulsifiers, disintegrating agents, binding agents, preservatives, buffering agents, lubricating agents, preservatives, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006). The use of a conventional excipient medium may be contemplated within the scope of the present disclosure.

In some aspects, the delivery system may be a therapeutic delivery system geared to sustained or controlled release formulation for example synthetic material depot, polymer depot, lipid depot, controlled release hydrogel depot, liquid crystalline depots, liposome depots, oil-based depots, and controlled release polymer depot. Depot formulations are one way to administer drugs, with a reduced dosing frequency and a simultaneous improvement of therapeutic efficacy and compliance of patients. In some aspects, these formulations are characterized by a slower release of the therapeutic with respect to a conventional release dosage form administered by the same route. In some aspects, the formulations are tunable and released at a predetermined rate within the therapeutic range for a specified period.

In some aspects, the compositions disclosed herein are formulated for administration into a subject in need thereof via one or more routes for example oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneously, sublingual, topically, trans buccal, transdermal, vaginal, in cremes, in lipid compositions, via a catheter, via a lavage, via continuous infusion, via infusion, via inhalation, via injection, via local delivery, or via localized perfusion. In some aspects, the pharmaceutical compositions are formulated for administration via injection. In some aspects, the pharmaceutical compositions are formulated as a unit dose. In some aspects, the formulation may further comprise excipients suitable for administration via the routes provided herein.

In some aspects, the compositions disclosed herein may further comprise excipients suitable for one or more suitable administration means. In some aspects, the compositions may be formulated as injectables, liquids, emulsions, suspensions, syrups, pills, caplets, creams, ointments, lotions, patches, solutions, suspensions, suppositories, lyophilizates, gels and capsules. Methods of making pharmaceutical compositions are well known in the art (See, e.g., Remington, The Science and Practice of Pharmacy, Alfonso R. Gennaro (Ed.) Lippincott, Williams & Wilkins (pub)). The pharmaceutical composition may also be formulated so as to facilitate timed, sustained, pulsed, or continuous release. The pharmaceutical composition may also be administered in a device, such as a timed, sustained, pulsed, or continuous release device.

In some aspects, the current disclosure also encompasses compositions comprising the polypeptides disclosed here in and a suitable delivery system. Any suitable delivery systems for polypeptides known in the art can be used herein. Examples of suitable delivery systems include but are not restricted to polymers, polyplexes, microspheres, lipids, lipidoids, lipoplexes, liposomes, microparticles, polymer nanoparticles, nanoparticles, lipid nanoparticles (LNPs), core-shell nanoparticles, solid lipid nanoparticles, metal nanoparticles, nanoparticle mimics, and any combination thereof. Details of some of the commonly used delivery systems are provided in the disclosure for delivery of polynucleotides but can be suitably adapted for delivery of polypeptides.

### III. Methods

In some aspects, the current disclosure also encompasses methods and applications that use the compositions disclosed herein. In some aspects, these compositions comprising a signal peptide provided herein or polynucleotide encoding them provided herein may be used for any suitable application. In some aspects, secretion of a protein of interest out of a cell may be beneficial in such an application. In some aspects, the current disclosure provides several signal peptides sequences, each of which may be suitable for use in one or more applications. In some aspects, these compositions can be used for non-therapeutic purposes. In some aspects, these compositions may be used for therapeutic applications.

In some aspects, the current disclosure provides a variety of engineered polynucleotides, engineered polypeptides, expression cassettes, viral vectors, expression vectors, host cells, suitable formulation that can enable secretion of a heterologous protein of interest out of a cell. Post-translation secretion of a polypeptide of interest from a cell may be desirable, for example, for systemic or organ specific delivery of a therapeutic, diagnostic, theragnostic and/or reporter polypeptide in a subject in need thereof. In some aspects, post-translation secretion of a polypeptide of interest may be desirable from a cell in a non-therapeutic application, for example in a laboratory experiment. In some aspects, post-translation secretion of a polypeptide of interest may be desirable for industrial application for easy production and isolation of a protein product. All such applications for the disclosed secretory signal peptides are envisaged in the current disclosure.

In some exemplary aspects, the current disclosure encompasses methods of diagnosis, prevention and/or treatment comprising administration of an effective amount of the compositions disclosed herein. In some aspects, the composition comprises a therapeutic polypeptide fused to the signal peptide disclosed herein, or a polynucleotide composition encoding the same. In some aspects, compositions (polypeptides or polynucleotides) corresponding to any therapeutic polypeptide for which secretion is desirable can be used in the disclosed method. For example, the compositions can be used in methods of ameliorating the effects of a disease, preventing a disease, treating a disease, or inhibiting the progress of a disease in a subject in need thereof. Such methods include inhibiting cell rolling, inflammation, autoimmune disease, metastasis, growth and/or replication of tumor cells or leukemia cells or increase in number of tumor cells in a subject having a tumor or leukemia cells in a subject having leukemia. In addition, such methods include increasing the mortality rate of tumor cells or leukemia cells, alter the susceptibility of diseased cells to damage by anti-disease agents, tumor cells to damage by anti-cancer agents, or leukemia cells to damage by anti-cancer agents. Such methods also include inhibiting or decreasing viral entry in cells. Such methods further include preventing or inhibiting cardiovascular diseases. In some aspects, the therapeutic polypeptide used in the method may have, for example, anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotics, anti-pruritic, cardiovascular, chemotherapeutic agents, hormonal activity. In some aspects, the therapeutic polypeptide is a protein replacement therapeutic, polypeptide therapeutic, vaccine, viral vaccine, secreted therapeutic, anti-cancer, anti-inflammatory, anti-viral, anti-microbial, hypocholestrolemic, anti-diabetic, or anti-fibrotic polypeptide. In some aspect the theraeutic polypeptide is an antibody. In some aspects, the polypeptide is a negative checkpoint regulator non-limiting examples of which include cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, and CD73. In some aspects, the polypeptide is a tumor antigen, non-limiting examples of which include alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand. In some aspects, the heterologous polypeptide is an antibody. In some aspects, the polypeptide is a Chimeric Antigen Receptor (CAR). In some aspects, the polypeptide is active as a vaccine. In some aspects, the method also encompasses use of a diagnostic polypeptide. In some aspects, the polypeptide is a theragnostic. In some aspects, the polypeptide is an antibody-based diagnostic. Generally, the polypeptide is labeled with a radionucleotide (such as 111In, 99Tc, 14C, 131, 3H, 32P or 35S) and specifically binds to a tumor antigen so that the tumor can be localized using immunoscintiography. In one aspect, polypeptides or fragments thereof bind to the extracellular domains of specific cancer biomarkers. Polypeptides for diagnostic use may be labeled with probes suitable for detection by various imaging methods. Methods for detection of probes include, but are not limited to, fluorescence, light, confocal and electron microscopy; magnetic resonance imaging and spectroscopy; fluoroscopy, computed tomography and positron emission tomography. Suitable probes include, but are not limited to, fluorescein, rhodamine, eosin and other fluorophores, radioisotopes, gold, gadolinium and other lanthanides, paramagnetic iron, fluorine-18 and other positron-emitting radionuclides.

Effective dosages/amounts and schedules for administering the composition may be determined empirically, and making such determinations is within the skill in the art. Those skilled in the art will understand that the dosage of the compositions disclosed herein that must be administered will vary depending on, for example, the subject that will receive the composition, the route of administration, the particular type of composition used, and other drugs being administered. For instance, for an anti-cancer therapeutic, a therapeutically-administered composition amount that arrests tumor growth, results in tumor shrinkage, and/or prevents the development of new tumors, compared to the disease course that would occur in the absence of the administration, is an efficacious dose. The compositions can be administered as a single dose or require repeated administration. In some aspects, the method of treatment may further comprise administration of additional treatments including additional drugs for example anti-inflammatory, analgesics, antimicrobials or therapies for example radiation therapy.

In some aspects, the subject in the method of treatment may include an animal, human or non-human, to whom treatment according to the methods of the present disclosure is provided. Human and veterinary applications are anticipated by the present disclosure. The term includes but is not limited to birds, reptiles, amphibians, and mammals, e.g., humans, other primates, pigs, rodents, such as mice and rats, rabbits, guinea pigs, hamsters, horses, cows, cats, dogs, sheep, chickens, and goats. In some aspects, the subject is a human. Both pediatric and adult subjects are included.

In some aspects, the current disclosure also encompasses methods of using the compositions provided herein for in vivo diagnostics. In some aspects, the composition comprises or encodes a diagnostic antibody. In some exemplary aspects, the method disclosed herein is used for tumor detection.

In some aspects, the current disclosure also encompasses methods of using the compositions disclosed herein in cell culture and tissue culture to enable secretion of a heterologous polypeptide. In some aspects, the method comprises contacting a cell with the compositions disclosed herein. In an exemplary aspect, the cell can be transfected with polynucleotide composition provided herein. In some aspects, the heterologous polypeptide can be a reporter polypeptide for example a fluorescent polypeptide or an antibody and the secreted polypeptide can be used for visualization using microscopy or other suitable techniques.

In some aspects, the current disclosure also encompasses methods of using the compositions disclosed herein for industrial applications. Secreted proteins provide several advantages for industrial scale production of products. In some exemplary aspect, purification of secreted proteins maybe easier and desirable than cell or tissue extraction of heterologous proteins. In some exemplary aspects, systemic secretion of a protein may be useful in food industry for imparting flavor to meat products. Disclosed herein are simply exemplary applications, that should not be considered limiting.

### IV. Kits

In some aspects, the compositions and methods provided herein can also be provided in the form of kits with instructions for use. In some aspects, the kits comprise at least a composition comprising a polynucleotide encoding a signal peptide provided herein, and optionally suitable substrates, reagents, buffers, diluents, cells, standards, containers and instructions for use. In some aspects, the kits may comprise at least a cell comprising the polynucleotide or polypeptide disclosed herein and optionally suitable substrates, reagents, buffers, diluents, cells, standards, containers, and instructions for use.

In some aspects, the article of manufacture or kit comprises a container and a label or package insert on or associated with the container. Suitable containers include, for example, microfuge tubes, bottles, vials, assay plates, strips, matrices etc. The containers may be formed from a variety of materials such as glass, plastic, paper etc. The kit may further include other materials desirable from a commercial and user standpoint, including other buffers, diluents, filters, needles, and syringes.

A “package insert” is used to refer to instructions customarily included in commercial packages of products, that contain information about usage etc.

Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### EXAMPLES

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Methods

SP-mCherry Plasmid (pDNA) Construction

To determine optimal signal peptide (SP), SP-modified mCherry plasmids were constructed. Briefly, SP-mCherry coding region were obtained directly by PCR with well-designed primers. Several SPs were selected, including hAlb (human Albumin, SEQ ID NO: 1). hApoB (human Apolipoprotein, SEQ ID NO: 2), gLuc (Gaussia luciferase, SEQ ID NO: 3) and hFVII (human Factor VII: SEQ ID NO: 4). The polypeptide sequences of the signal sequence fused to mcherry are provided as SEQ ID NO: 62-66 and described in Table 1. Enzyme digested SP-mCherry products were cloned into pCS2-MT vector based on standard protocols. After validation by sequencing, SP-mCherry plasmids were ready for in vitro screening.

TABLE 1

| | | SEQ | | Predicted |
|---|---|---|---|---|
| Sample ID | AA sequence (SP + mCherry) | ID NO: | SP Sources | SP (7) |
| WT- | MVSKGEEDNMAIIKEFMRFKVHMEG | 55 | N/A | No |
| mCherry | SVNGHEFEIEGEGEGRPYEGTQTAK | | | |
| | LKVTKGGPLPFAWDILSPQFMYGSKA | | | |
| | YVKHPADIPDYLKLSFPEGFNWERV | | | |
| | MNFEDGGVVTVTQDSSLQDGEFIYK | | | |
| | VKLRGTNFPSDGPVMQCRTMGWEA | | | |
| | STERMYPEDGALKGEIKQRLKLKDG | | | |
| | GHYDAEVKTTYKAKKPVQLPGAYNV | | | |
| | DIKLDILSHNEDYTIVEQYERAEGRHS | | | |
| | TGGMDELYK | | | |
| NC- | MEDAKNIKKGPAPFYPLEDGMVSKG | 62 | GenBank: | No |
| mCherry | EEDNMAIIKEFMRFKVHMEGSVNGH | | CAB91857.1 | |
| | EFEIEGEGEGRPYEGTQTAKLKVTKG | | | |
| | GPLPFAWDILSPQFMYGSKAYVKHP | | | |
| | ADIPDYLKLSFPEGFNWERVMNFED | | | |
| | GGVVTVTQDSSLQDGEFIYKVKLRGT | | | |
| | NFPSDGPVMQCRTMGWEASTERMY | | | |
| | PEDGALKGEIKQRLKLKDGGHYDAE | | | |
| | VKTTYKAKKPVQLPGAYNVDIKLDILS | | | |
| | HNEDYTIVEQYERAEGRHSTGGMDE | | | |
| | LYK | | | |
| hAlb- | MKWVTFISLLFLFSSAYSMVSKGEED | 63 | GenBank | Yes |
| mCherry | NMAIIKEFMRFKVHMEGSVNGHEFEI | | AAA98797.1 | |
| | EGEGEGRPYEGTQTAKLKVTKGGPL | | | |
| | PFAWDILSPQFMYGSKAYVKHPADIP | | | |
| | DYLKLSFPEGFNWERVMNFEDGGVV | | | |
| | TVTQDSSLQDGEFIYKVKLRGTNFPS | | | |
| | DGPVMQCRTMGWEASTERMYPEDG | | | |
| | ALKGEIKQRLKLKDGGHYDAEVKTTY | | | |
| | KAKKPVQLPGAYNVDIKLDILSHNED | | | |
| | YTIVEQYERAEGRHSTGGMDELYK | | | |
| hApoB- | MDPPRPALLALLALPALLLLLLAGARA | 64 | NCBI Ref | Yes |
| mCherry | MVSKGEEDNMAIIKEFMRFKVHMEG | | NP_000375.3 | |
| | SVNGHEFEIEGEGEGRPYEGTQTAK | | | |
| | LKVTKGGPLPFAWDILSPQFMYGSKA | | | |
| | YVKHPADIPDYLKLSFPEGFNWERV | | | |
| | MNFEDGGVVTVTQDSSLQDGEFIYK | | | |
| | VKLRGTNFPSDGPVMQCRTMGWEA | | | |
| | STERMYPEDGALKGEIKQRLKLKDG | | | |
| | GHYDAEVKTTYKAKKPVQLPGAYNV | | | |
| | DIKLDILSHNEDYTIVEQYERAEGRHS | | | |
| | TGGMDELYK | | | |
| gLuc- | MGVKVLFALICIAVAEAMVSKGEEDN | 65 | GenBank: | Yes |
| mCherry | MAIIKEFMRFKVHMEGSVNGHEFEIE | | BAR71165.1 | |
| | GEGEGRPYEGTQTAKLKVTKGGPLP | | | |
| | FAWDILSPQFMYGSKAYVKHPADIPD | | | |
| | YLKLSFPEGFNWERVMNFEDGGVVT | | | |
| | VTQDSSLQDGEFIYKVKLRGTNFPSD | | | |
| | GPVMQCRTMGWEASTERMYPEDGA | | | |
| | LKGEIKQRLKLKDGGHYDAEVKTTYK | | | |
| | AKKPVQLPGAYNVDIKLDILSHNEDYT | | | |
| | IVEQYERAEGRHSTGGMDELYK | | | |
| hFVII- | MVSQALRLLCLLLGLQGCLAMVSKG | 66 | GenBank: AA | Yes |
| mCherry | EEDNMAIIKEFMRFKVHMEGSVNGH | | P33841.1 | |
| | EFEIEGEGEGRPYEGTQTAKLKVTKG | | | |
| | GPLPFAWDILSPQFMYGSKAYVKHP | | | |
| | ADIPDYLKLSFPEGFNWERVMNFED | | | |
| | GGVVTVTQDSSLQDGEFIYKVKLRGT | | | |
| | NFPSDGPVMQCRTMGWEASTERMY | | | |
| | PEDGALKGEIKQRLKLKDGGHYDAE | | | |
| | VKTTYKAKKPVQLPGAYNVDIKLDILS | | | |
| | HNEDYTIVEQYERAEGRHSTGGMDE | | | |
| | LYK | | | |

In Vitro SP Screening by pDNA Transfection

To perform SPs screening, pDNA transfection was executed in cells. Hela and Huh7 cells were seeded into 96-well plate with 1×10⁴ cells per well. After 24 hours, cells were treated by Lipo2k-pDNA formulations with 50 ng pDNA per well. At day 1, day 2 and (or) day 3 after treatment, cells were imaged immediately by Keyence Scope. Meanwhile, cell lysates and medium were further collected, then mCherry signal was quantified by plater reader. To observe mCherry signal clearly, cell lysates and medium were further transferred into EP tubes and imaged by IVIS Lumina system. To test subcellular signal distribution, confocal microscopy was used. Huh7 cells were treated as described above, after 3 days, cells were washed three timed by 1×PBS, stained by Hoechst 33342 and imaged by confocal microscopy.

mRNA Synthesis

All mRNAs this work used were produced by in vitro transcription (IVT) as described before (Cheng et al. 2020). Briefly, linear pDNA with optimized 5′ (3′)-untranslated regions (UTR) and poly A sequences were obtained first by enzyme digestion, then IVT reactions were prepared with standard protocols with N1-methylpseudouridine-5′-triphosphate modification. Finally, mRNA was capped (Cap-1) by Vaccinia Capping Enzyme and 2′-O-methyltransferase (NEB).

mRNA-Nanoparticle Formation

mRNA-loaded LNP formulations were formed using the ethanol dilution method described previously (Cheng et al. 2020). The liver-targeted mRNA formulation (mDLNP) and tissue selective SORT LNPs were developed and reported (Cheng et al. 2018, 2020). Briefly, all lipids with specified molar ratios were dissolved in ethanol and RNA was dissolved in 10 mM citrate buffer (pH 4.0) first. Then the two solutions were rapidly mixed at an aqueous to ethanol ratio of 3:1 by volume (3:1, aq.:ethanol, vol:vol) to satisfy a final weight ratio of 40:1 (total lipids:mRNA). After incubation 10 min at room temperature, the mRNA LNPs formulations were added immediately into cells or dialyzed against PBS for 2 h for in vivo experiments.

In Vitro mCherry Secretion Driven by Optimal hFVII-SP Verified with mRNA Formulation

hFVII-mCherry mRNA was transfected in several kinds of cell lines, including Huh7, 293T, Hela, A549 and IGROV1. Cells were seeded into 96-well plates with 1×10⁴ cells per well for 24 hours. The mRNA mDLNPs formulations was prepared as described above, then cells were treated with various mRNA doses (0 to 750 ng per well) and time points (24 h to 72 h). At given time, cells were imaged directly by Keyence Scope, and then mCherry signal in medium and cell lysates were quantified by plater reader. WT-mCherry mRNA formulation was used as a control group.

In Vivo mCherry Secretion Driven by Optimal hFVII-SP Verified with mRNA Formulation

All animal experiments were approved by the Institution Animal Care and Use Committees of The University of Texas Southwestern Medical Center and were consistent with local, state and federal regulations as applicable. C57BL/6 mice were obtained from the UTSW Mouse Breeding Core Facility. For luciferase mRNA testing, mDLNPs, SORT LNPs (liver-, lung- and spleen-) formulations were I.V. injected into mice with dose of 0.1 mg/kg mRNA. At 3 hours, mice were injected with D-Luciferin (150 mg/kg, intraperitoneal (IP)) and imaged by an IVIS Lumina system (Perkin Elmer). To test mCherry secretion, hFVII-mCherry mRNA was encapsulated into mDLNPs and I.V. injected into mice with dose of 0.5 mg/kg mRNA. At different time points (2 h, 6 h, 24 h, 30 h, 48 h, 55 h and 72 h), serum was separated and mCherry signal was quantified by plate reader. Meanwhile, tissues were imaged by IVIS to confirm mCherry secretion in the blood. PBS and WT-mCherry formulations treated groups were used as control. To further test mCherry secretion, SORT LNPs, liver-, lung- and spleen-targeted, were used to deliver hFVII-mCherry mRNA (0.5 mg/kg) into liver, lung and spleen, respectively. And tissues were imaged by IVIS at 24 hours.

Cytotoxicity Rescue of hFVII-Enbrel mRNA

L929 cells were used to evaluate TNF-α mediated cytotoxicity. Both mouse TNF-α (mTNF-α) and human TNF-α (hTNF-α) were selected. Cells were seeded into 96-well plate with density of 1×10⁴ cells per well for 24 h. Medium was replaced with 180 μl fresh medium contained the Actinomycin and TNF-α to make the Actinomycin final concentration of 1 μg/ml and TNF-α concentrations of 0 to 0.1 ng/ml. After incubation another 24 h, cell viability was detected by CellTiter-Glo kit based on the standard protocol.

To evaluate cytotoxicity rescue of hFVII-Enbrel mRNA formulations, cells were pretreated with hFVII-Enbrel mRNA mDLNPs for two days before challenging with TNF-α. Dose-dependent rescue for both mRNA and TNF-α was tested, respectively. For dose dependent rescue of mRNA formulations, mRNA doses of 0 ng/ml to 1.25 ng/ml per well were tested, then challenged with TNF-α of 0.1 ng/ml for 24 h. For dose dependent rescue of TNF-α, transfected mRNA concentration of 0.4 ng/ml was fixed, then challenged with TNF-α of 0 ng/ml to 5 ng/ml.

To further verify the rescue effects that was from secreted Enbrel in medium, the cytotoxicity rescue pretreated with functional medium was measured. Cells were seeded and treated as described above, with mRNA concentrations of 0 ng/ml to 1.25 ng/ml, then collected the medium and transferred into a new 96-well plate with attached L929 cells. At the same time, the new plate was challenged with TNF-α (0.1 ng/ml) and Actinomycin (1 μg/ml). After another 24 h, cell viability was detected.

### Pharmacokinetics Study

Male C57BL/6 mice, weight of 20 g, were randomly divided into groups. Enbrel protein and hFVII-Enbrel mRNA mDLNPs formulation were I.V. injected with dose of 0.5 mg/kg. At time-points of 2 h to 216 h, serum was collected and Enbrel in serum was quantified by ELISA kit (MyBioSource).

### Psoriasis Therapy

For the imiquimod-induced psoriasiform hyperplasia model, 8 week old female C57BL/6 mice were shaved and chemically depilated with Nair (indicated day 1). At day 3, the mice were I.V. injected with hFVII-Enbrel formulation with dose of 0.5 mg/kg. Then the shaved dorsal-skin samples were treated topically with 60 mg of Aldara cream (5% imiquimod) (Aldara, 3 M Pharmaceuticals) daily for a total of 5 days. At day 9, the whole body were pictured by camera to show difference between groups, herein the Lanolin+PBS treated mice and imiquimod+mCherry mDLNPs treated mice were control groups. The dorsal skin was harvested at end point to measure the thickness by H&E staining and the cell proliferation was analyzed by immunohistochemistry of Ki-67 and Gr-1.

### Assessment of PDL1 Expression

MC38, B16F10 cell lines were used to study in vivo tumor immunotherapy. To assess the PDL1 expression in cell membrane, flow cytometry was used. Cells were seeded into 6-well plate with density of 3×10⁵ cells per well for 24 h. Cells were incubated with IFN-γ (100 ng/ml) for additional 24 h, after staining by primary anti-PDL1 antibody and Alexa Fluor 647 labelled the second antibody, PDL1 expression was analyzed by flow cytometry. Isotype antibody-stained cells were used for gating.

### Tumor Immunotherapy

MC38 or MC38-Luc (stably expressing luciferase) cells were grown in DMEM medium with 10% FBS. At day 0, total 1×10⁶ cells in 100 ul PBS were S.C. injected into right flank of C57BL/j mice. The hFVII-antiPDL1 mRNA mDLNPs formulation was I.V. continuously injected with dose of 0.5 mg/kg mRNA at day 3 for 3 times per every 4 days. For MC38 model, tumors were measured, and survival curve was monitored. For MC38-Luc model, luciferase expression was continuously captured by IVIS at day 3, day 10, day 24 and day 32, and luciferase signal was quantified by IVIS software. For B16F10-Luc model, total 4×10⁵ cells were S.C. injected, and mRNA formulation was I.V. injected as the same as described above. Luciferase signal, tumor size and survival were monitored from day 0 to day 32. For both tumor models, the mCherry mDLNPs formulation was used as control group. Tumor was measured with a digital caliper and the tumor sizes were calculated using the formula: volume=0.5×length×width. Mice were sacrificed and recorded as death when the tumor volume reached 1500 cm³ or larger.

### Example 1: Selection of Signal Peptides for Therapeutic Applications

Initially, multiple naturally occurring signal peptides (SP) were screened to test their ability to drive secretion of reporter proteins in cell cultures. Towards this end several different SPs from three known endogenously secreted proteins (albumin, hAlb; apolipoprotein B, hApoB; and Factor VII, hFVII) and one known synthetic secreted protein (Gaussia luciferase; gLuc) along with a negative control (NC) SP leader sequence were cloned into a pCS2-MT plasmid backbone directly upstream from a reporter mCherry mRNA sequence.

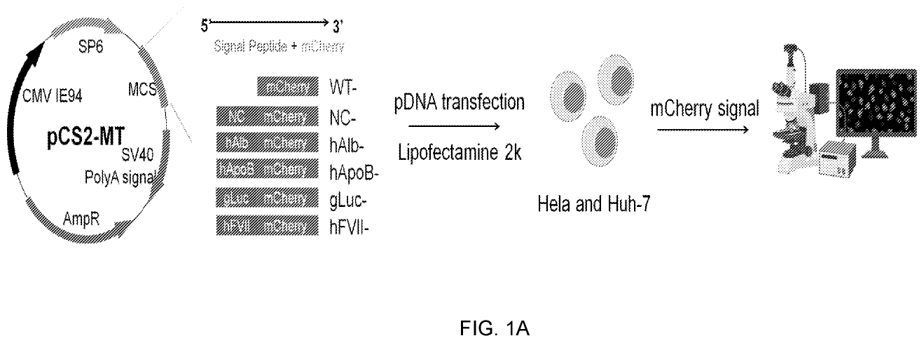

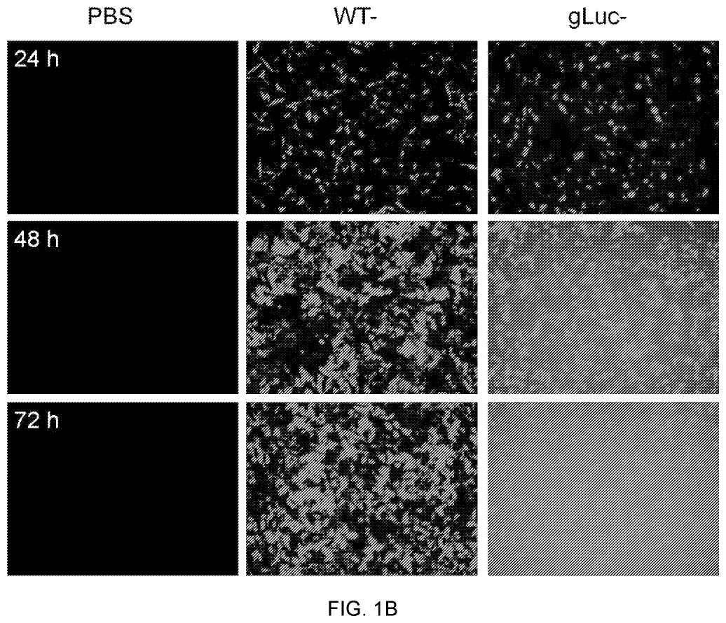

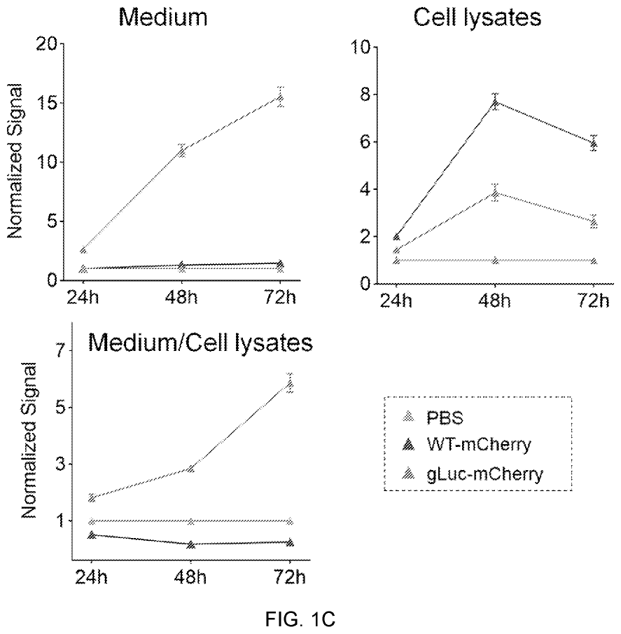

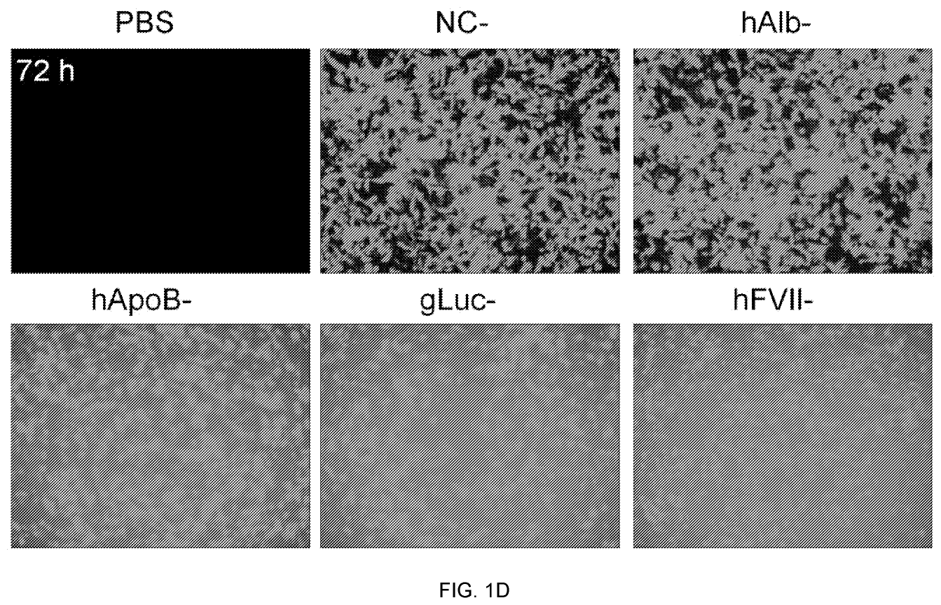

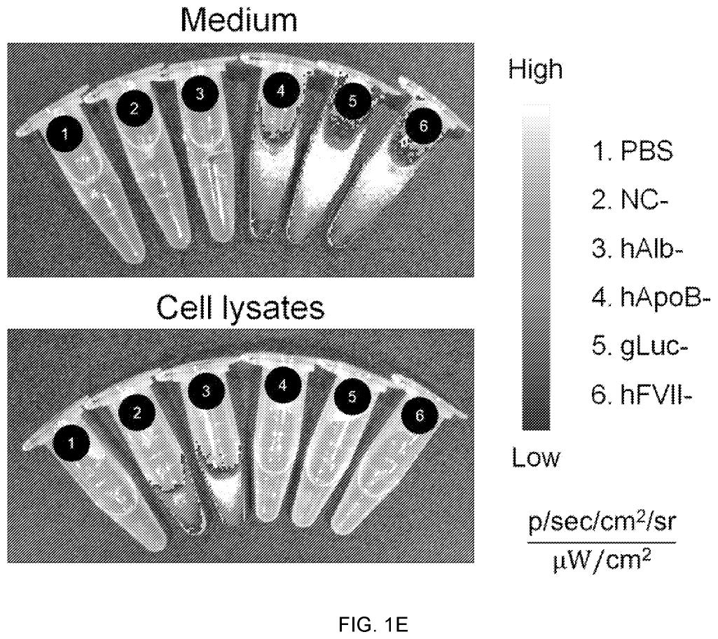

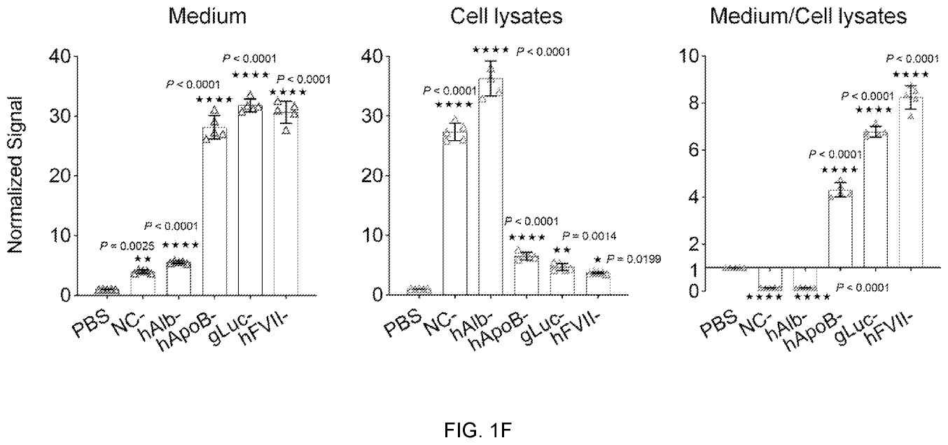

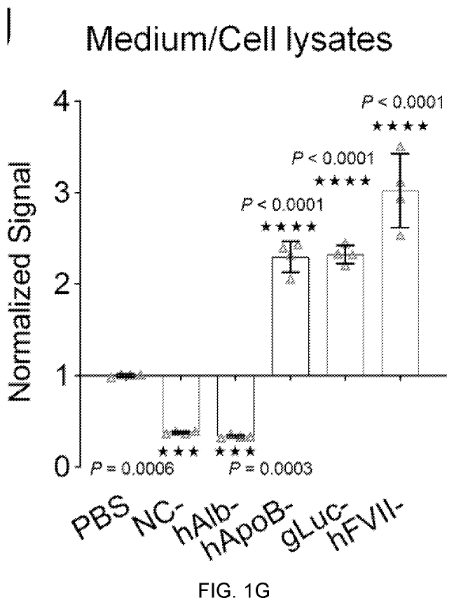

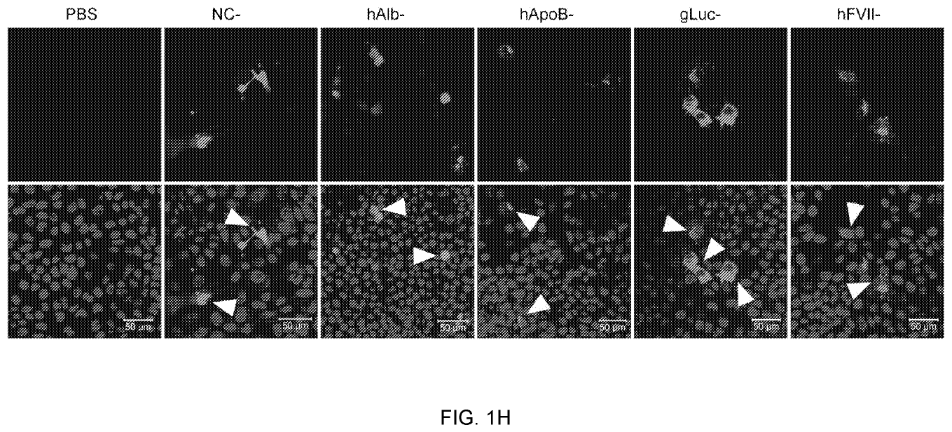

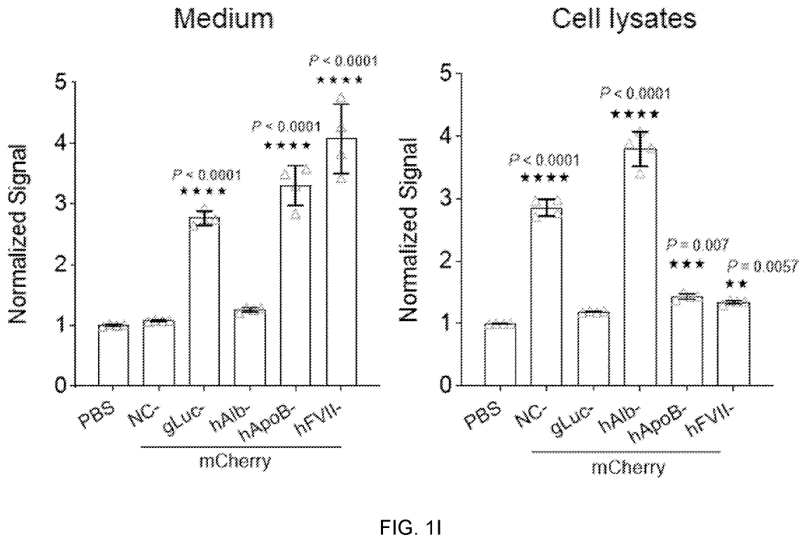

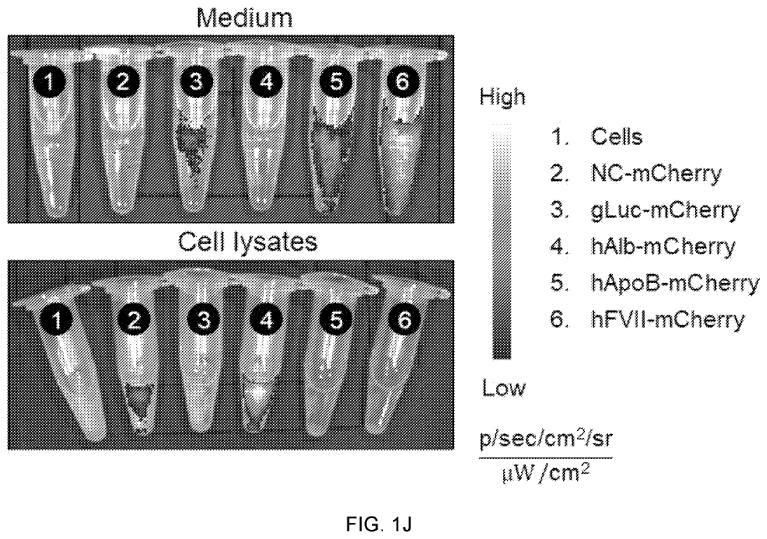

The construct was preceded by the SP6 promoter and an optimized 5′ UTR, and followed with an optimized 3′ UTR and polyA tail (FIG. 1A). Initially, Hela cells were transfected with wild type (WT) mCherry pDNA containing no SP and gLuc-mCherry pDNA via Lipofectamine2000, and both intracellular and extracellular fluorescence were quantified via fluorescent microscopy at 24-, 48-, and 72-hours post transfection, wherein the gLuc SP induced high levels of mCherry secretion into media (FIG. 1B). Additionally, mCherry protein content present in cell medium and cell lysate were quantified via a fluorescence plate reader individually at 24-, 48-, and 72-hours revealing an increase in mCherry secretion into medium over time along with an enhanced medium to cell lysate ratio of mCherry fluorescence in the gLuc SP group (FIG. 1C). The set of SPs was then expanded to include a negative control (scramble sequence), hAlb, hApoB, and hFVII, in addition to gluc. Hela cells were again transfected with the pDNA constructs using Lipofectamine2000. Images taken 72 h post transfection via fluorescence microscopy and IVIS demonstrated that the SPs hApoB, gLuc, and hFVII all generated high levels of mCherry protein secretion, while the NC and hAlb constructs effectively mediated intracellular mCherry expression but did not promote significant extracellular secretion (FIG. 1D-F). The same set of SPs were evaluated in the liver cancer cell line Huh7 wherein the observed trends of mCherry secretion in Hela cells persisted (FIG. 1I-J); however, transfected cells were also analyzed using confocal microscopy which revealed morphological differences in mCherry signal between SPs driving extracellular secretion of mCherry and those that promoted only intracellular mCherry expression (FIG. 1G-H). Overall, it was determined that the hFVII SP was able to cultivate the highest levels of protein secretion across both cell lines.

### Example 2: Organ Delivery of mRNA Encoding Signal Peptides and Secretion of Encoded Polypeptide

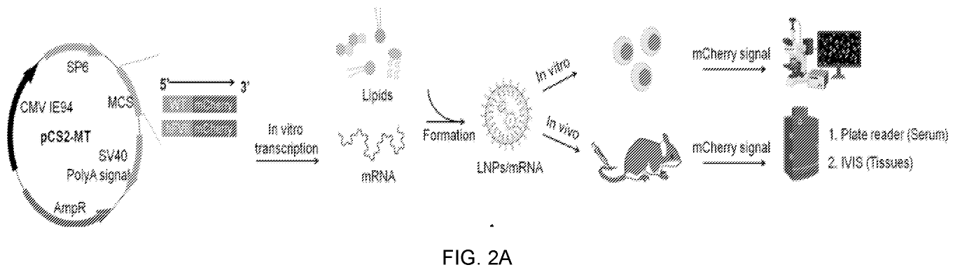

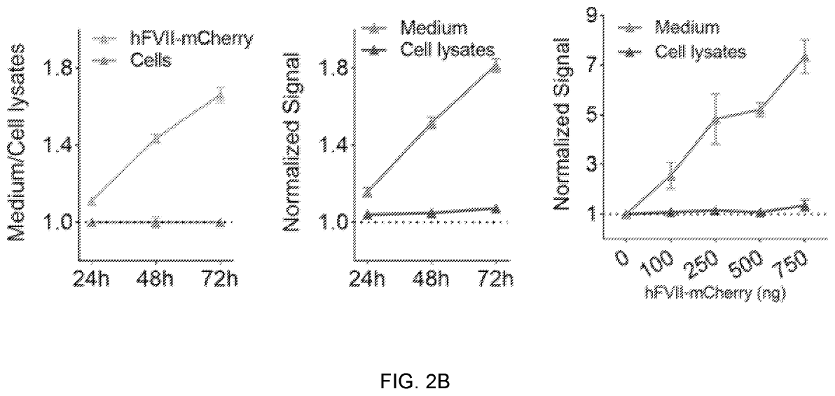

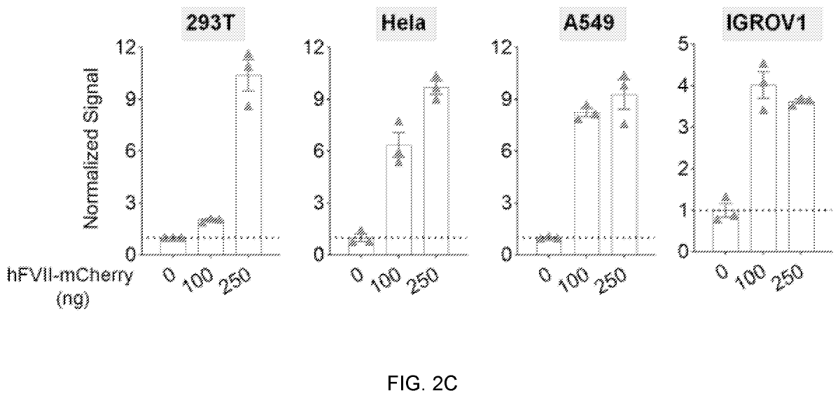

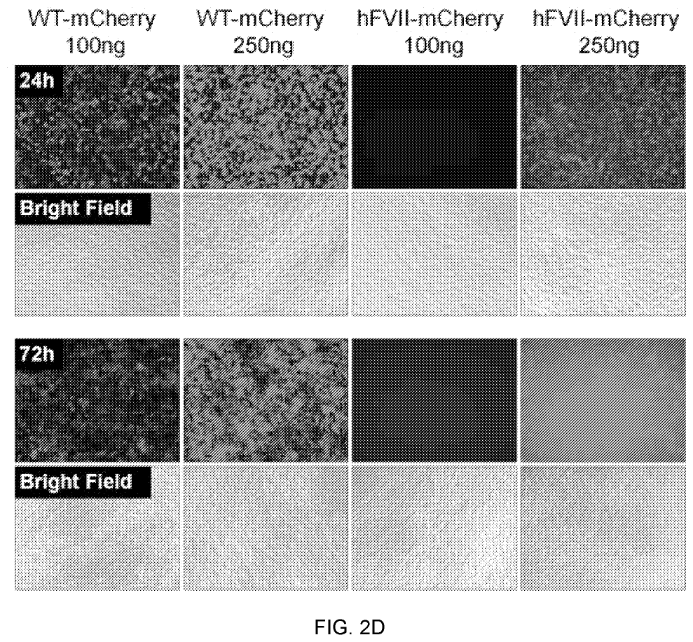

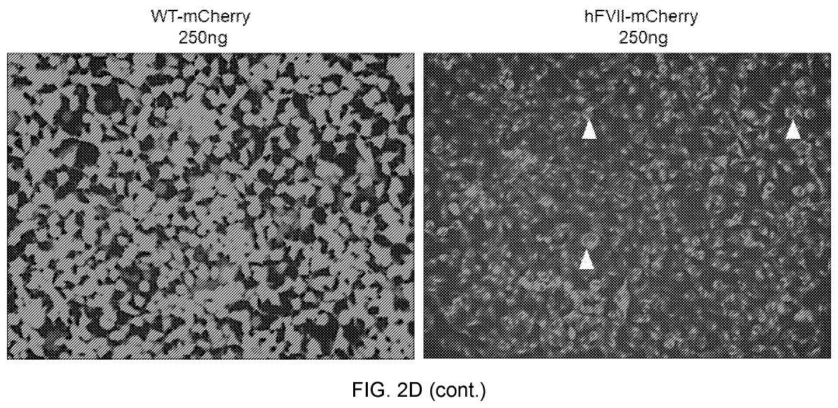

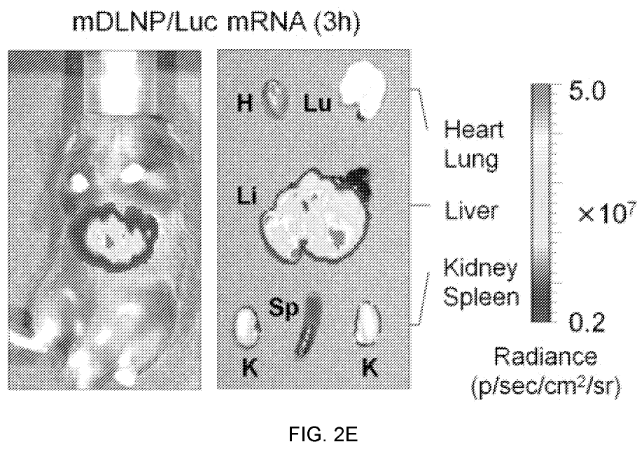

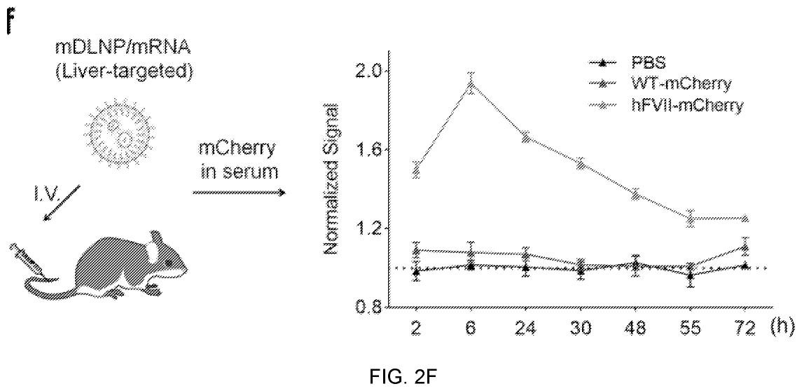

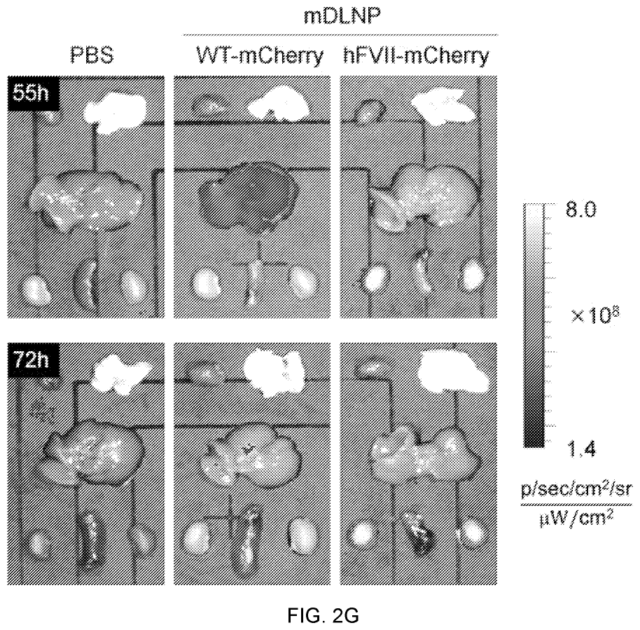

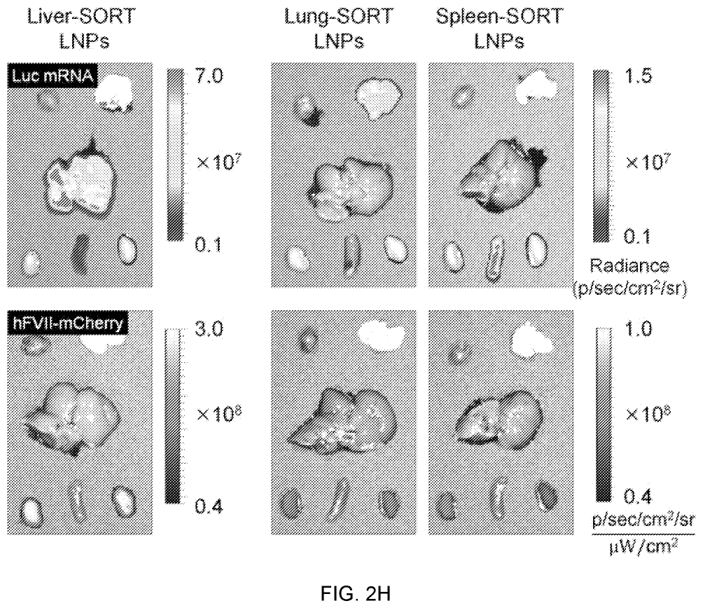

Based on the above observation, it was deemed interesting to see if mRNA containing an integrated SP sequence would yield similar observations to what was exhibited with pDNA. In pursuit of this query, hFVII-mCherry mRNA from the FVII-mCherry-pCS2-MT plasmid via in vitro transcription (IVT) were generated (FIG. 2A). A mDLNP lipid nanoparticle was tested for use as a initial carrier for the RNA. Transfection of multiple different cell lines with mDLNPs containing FVII-mCherry mRNA demonstrated that protein export into medium positively correlates with time post-transfection as well as dose, with greater fluorescence signal intensity being observed at longer time intervals and higher dosages across cell lines (FIG. 2B-D).

Next liver-targeting mDLNPs were tested to deliver FVII-mRNA in mice to the liver. mDLNPs liver targeting capability was first validated using luciferase mRNA-loaded mDLNPs delivered via intravenous injection with analysis via IVIS showing bright luminescence 6 h post injection (FIG. 2E). From there, hFVII-mCherry mRNA and WT-mCherry mRNA were encapsulated into mDLNPs and administered intravenously (IV) into mice. To determine whether mDLNPs containing hFVII mCherry mRNA were able to generate secretion of mCherry into systemic circulation thereby allowing the liver to act as protein factory, blood was collected at 2-, 6-, 24-, 30-, 48-, 55-, and 72-hours post injection.

Fluorescence analysis of serum from the mice revealed that no secretion occurred in the WT-mCherry group, but that mCherry signal was present in serum at all timepoints for the FVII-mCherry group, with peak concentration occurring 6 hours post injection (FIG. 2F). IVIS images were also taken at 55 h and 72 h post injection for the WT-mCherry and hFVII-mCherry injected mice. Interestingly, in the WT group mCherry fluorescence can be seen in the liver at 55 h with complete dissipation of signal by 72 h. However, at both time points, bright mCherry fluorescence can be seen in the kidneys of mice injected with mDLNPs containing hFVII mCherry mRNA, indicative of mCherry protein that is being cleared from systemic circulation via renal filtration (FIG. 2G).

Next it was tested if liver, as well as extrahepatic organs including lung and spleen were capable of promoting mCherry secretion when targeted and transfected with hFVII mCherry mRNA-loaded liver, lung, and spleen SORT LNP formulations, respectively. To investigate this hypothesis further, the targeting capabilities of SORT technology with luciferase-loaded liver, lung, and spleen SORT LNPs administered IV were first confirmed.

Indeed, bright luminescence was present in each of the respective organs following injection with the corresponding SORT LNPs. Each SORT LNP formulation was then loaded with hFVII mCherry mRNA and IV injected into mice which were imaged via IVIS 24 h following injection. Due to inclusion of the hFVII SP, mCherry protein was secreted by all of the SORT LNPs from each respective tissue. The signal in all cases was present in the kidneys for all groups, indicating systemic clearance of the mCherry protein through the kidney. The signal was notably higher in the Liver SORT group. Taken together, it can be determined that the liver, lung, and spleen are all capable of mediating intracellular mCherry protein manufacturing and extracellular protein secretion into systemic circulation following transfection with hFVII mCherry mRNA loaded Liver, Lung, and Spleen SORT LNPs (FIG. 2H).

### Example 3: Psoriasis Disease Treatment by hFVII-Enbrel mRNA Formulations

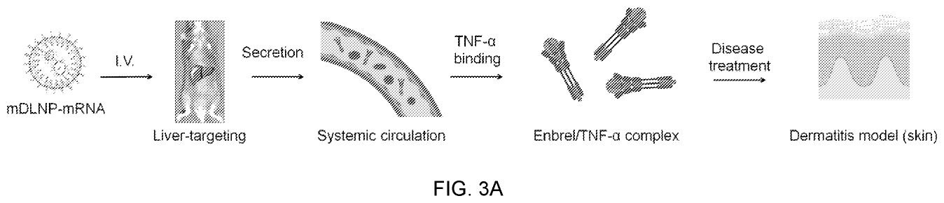

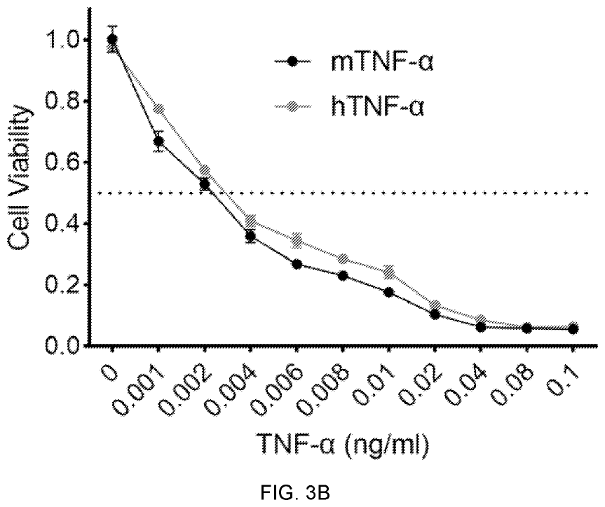

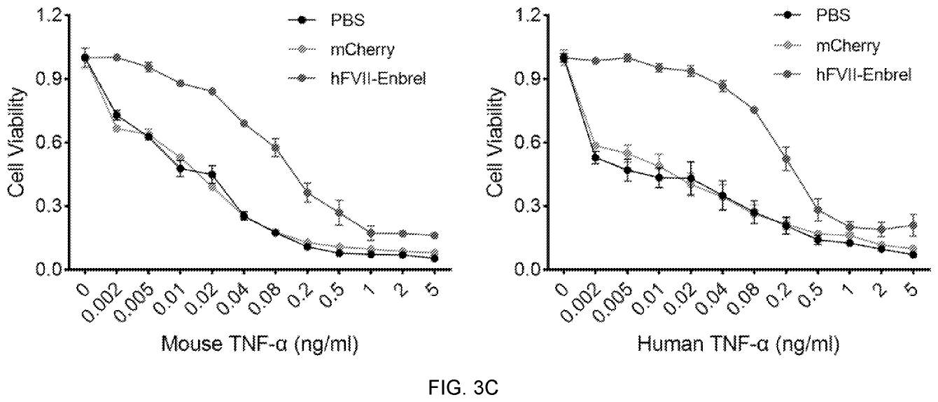

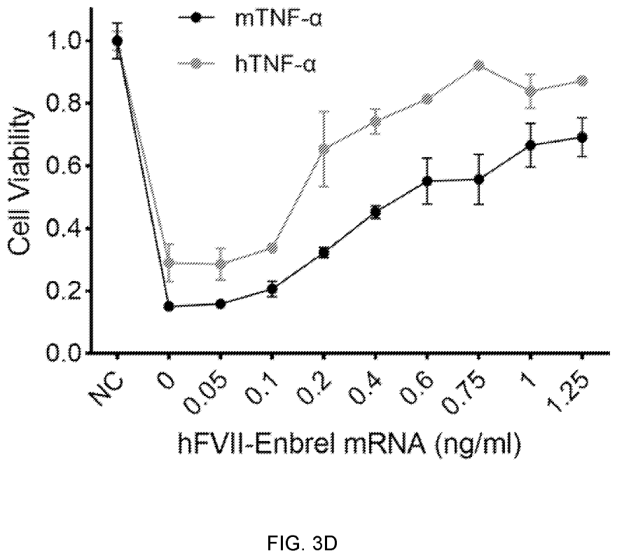

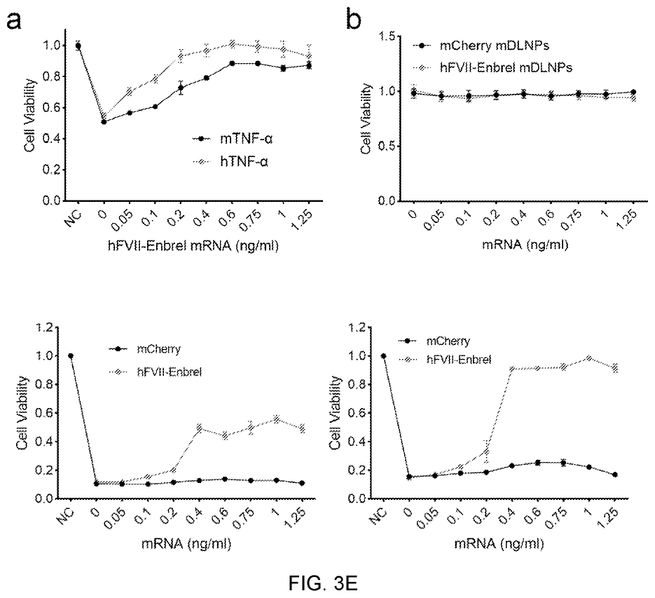

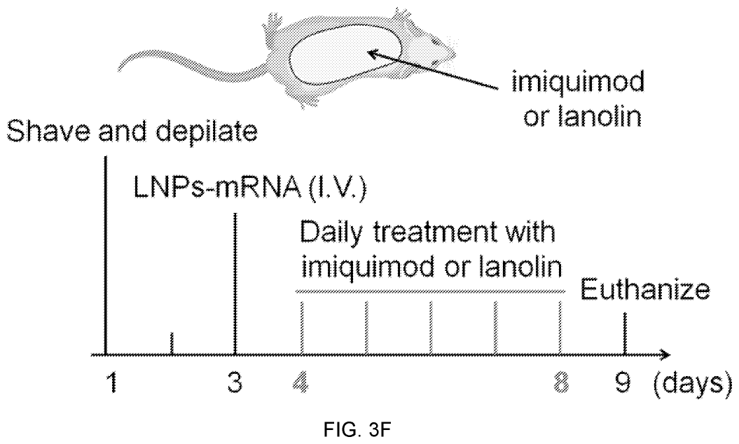

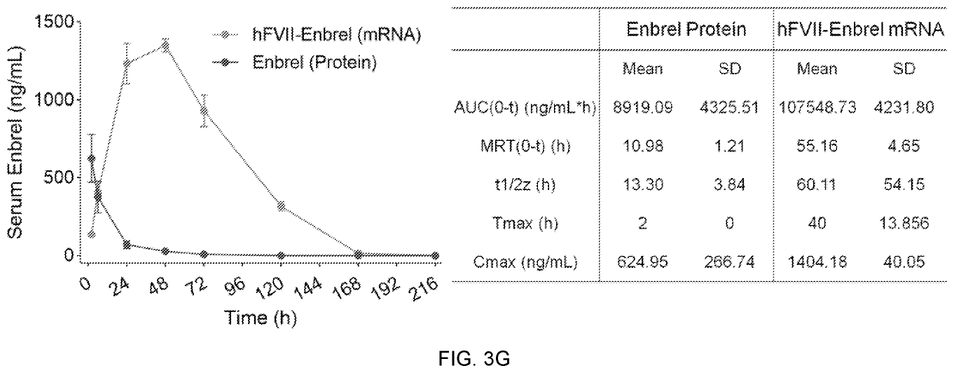

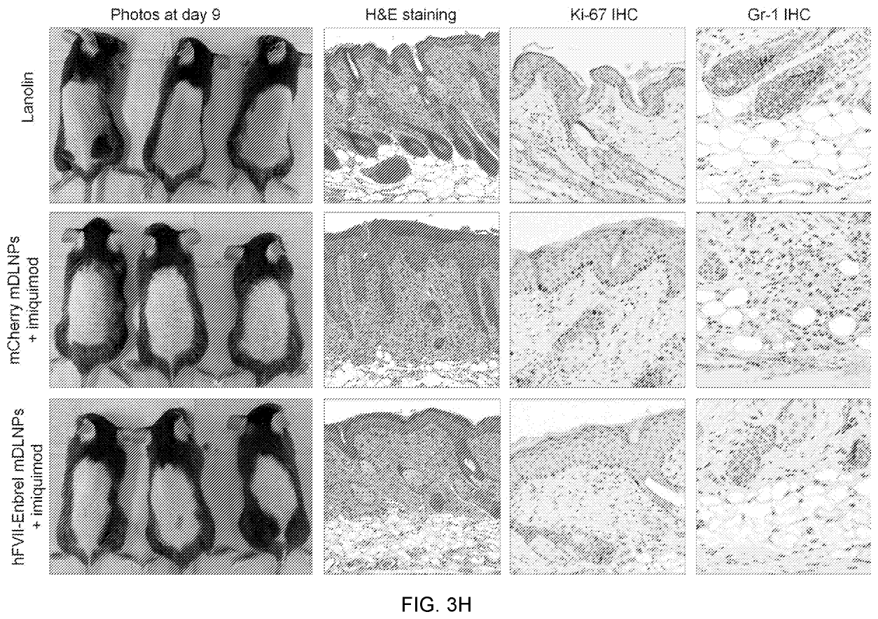

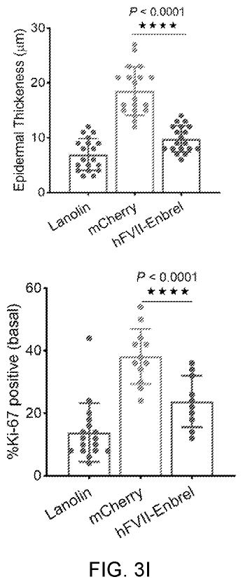

To test the use of the signal peptides in therapeutic context, mRNA encoding hFVII SP followed by the therapeutic synthetic dimeric fusion protein, Enbrel (Etanercept), and encapsulated it into mDLNPs were tested in L929 cells and an imiquimod-induced psoriasis in vivo model (FIG. 3A). L929 cells were first treated with mouse and human sourced TNF-α at doses of 0.001 to 0.1 ng/ml. At concentrations of just 0.02 ng/ml, less than 20% of cells remain viable (FIG. 3B). Next, cells were pre-treated with 80 ng of hFVII-Enbrel mRNA loaded LNPs and subsequently challenged via administration of either mouse or human TNF-α at doses ranging from 0.002 ng/ml to 5 ng/ml 48 h post hFVII-Enbrel mRNA LNP treatment. In both treatment groups, pre-treatment with hFVII-Enbrel mRNA mDLNPs resulted in cell viability remaining significantly higher across TNF-α dose ranges when compared with PBS and mCherry mRNA controls (FIG. 3C). Additionally, L929 cells were pre-treated for 48 h with hFVII Enbrel mRNA mDLNPs at dosages ranging from 0.05 ng/ml to 1.25 ng/ml and then dosed with 0.1 ng/ml of either mouse or human TNF-α. As expected, there was a dose-dependent rise in cell viability with respect to increasing pre-treatment concentrations of hFVII Enbrel mRNA (FIG. 3D). Finally, L929 cells that received 0.1 ng/mL of either mouse or human TNF-α were able to be rescued in a dose-dependent fashion after treatment with medium from cells pre-treated with hFVII Enbrel mRNA mDLNPs at doses of 0.05 ng/ml to 1.25 ng/ml (FIG. 3E upper panels), with viability restoration reaching nearly 100% in the human TNF-α group at an mRNA dose of just 0.4 ng/ml (FIG. 3E, lower panels).

An in vivo imiquimod induced psoriasis model was designed to assess the therapeutic potential of mDLNP-mediated hFVII Enbrel mRNA. Mice were initially shaved and depilated and then separated into three groups: a negative control group receiving no LNPs and a small dose of Lanolin cream on days 4-8; and two experimental groups wherein one group was administered hFVII Enbrel mRNA mDLNPs via IV and the other group was given mCherry mDLNPs via IV, both 3 days after shaving and depilating. In the two experimental groups, the mice were administered imiquimod on days 4-8 to induce a psoriatic-like phenotype. Serum pharmacokinetics of Enbrel were first assessed via blood draws and an Enbrel ELISA following IV injection with 0.5 mg/kg hFVII Enbrel mDLNPs or Enbrel protein. In mice injected with Enbrel protein, peak serum concentration occurred 2 h post injection and quickly diminished thereafter. However, in the group injected with hFVII Enbrel mRNA mDLNPs, serum concentration continued to rise until 48 h post injection and remained detectible until 168 h post injection. hFVII Enbrel mRNA mDLNPs demonstrated >10-fold increase in AUC and >20-fold increase in Tmax (h) when compared with Enbrel protein (107548.73, +/−4321.8 vs. 8919.09+/−4325.51; and 2 h vs. 40 h, respectively) (FIG. 3G). In all mice, H&E staining and Ki-67 and Gr-1 IHC staining was performed for dermal histological cross-sections. In addition to the obvious inflammation depicted in the mouse images, epidermal thickness and Ki-67 positive cells are also objectively elevated in the mCherry imiquimod treated group when compared with the Lanolin control group and the hFVII Enbrel mRNA imiquimod group, thereby suggesting that hFVII Enbrel mRNA mDLNPs are able to provide significant therapeutic benefit in an in vivo mouse psoriasis model (FIG. 3H-I).

### Example 4: Tumor Immunotherapy by hFVII-Anti-PDL1 mRNA

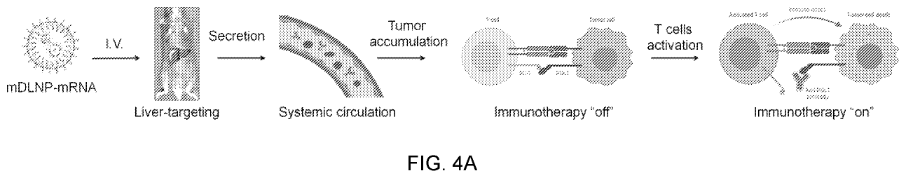

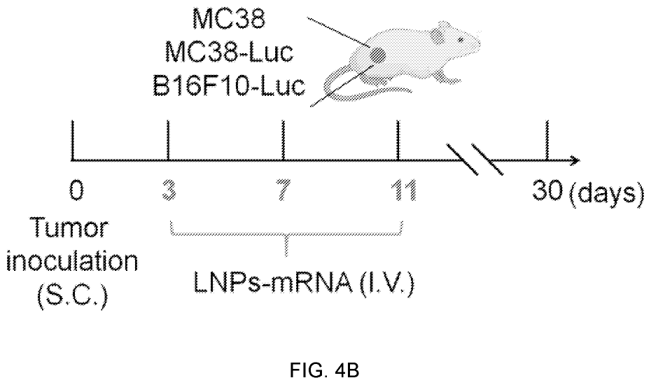

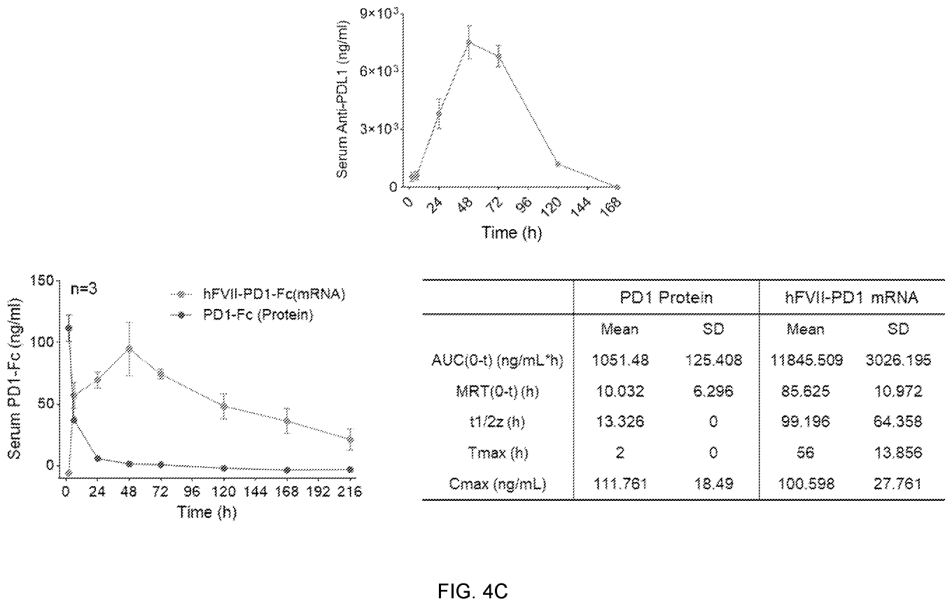

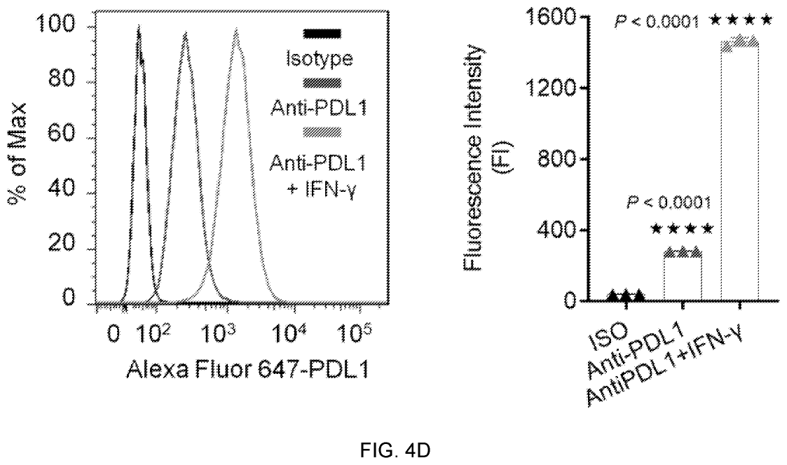

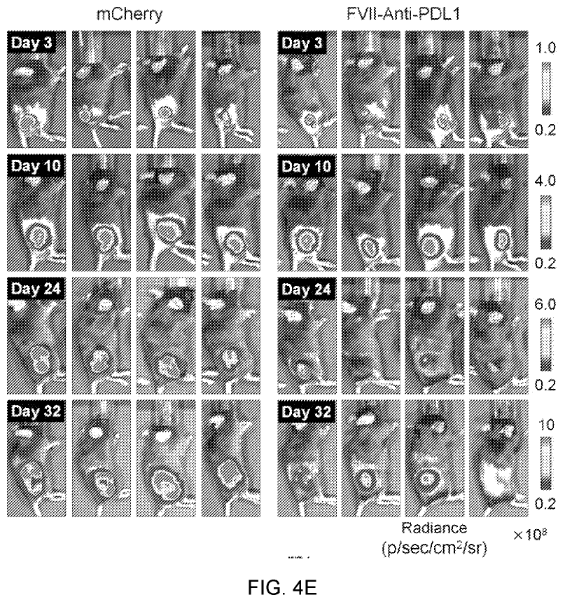

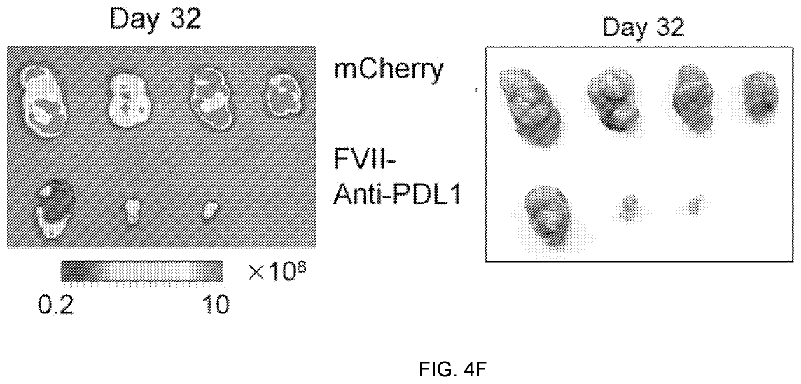

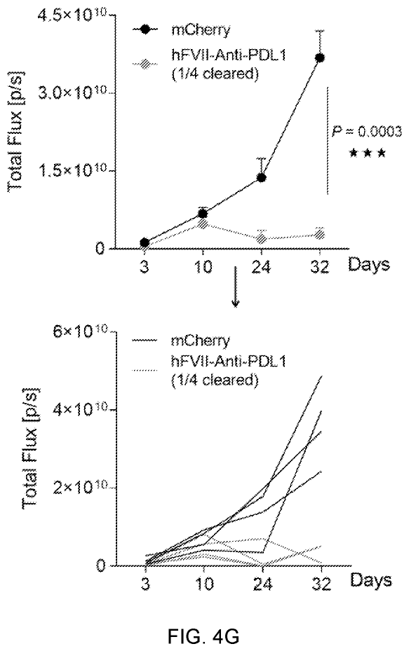

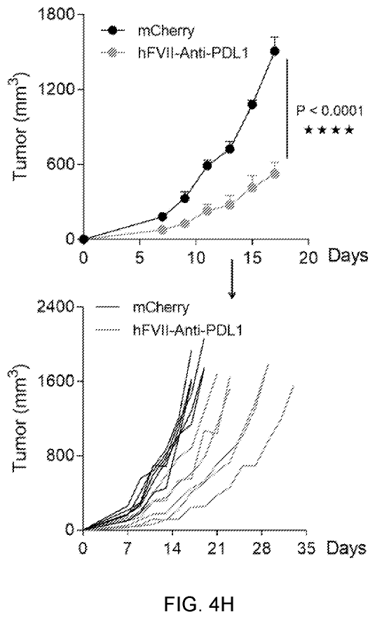

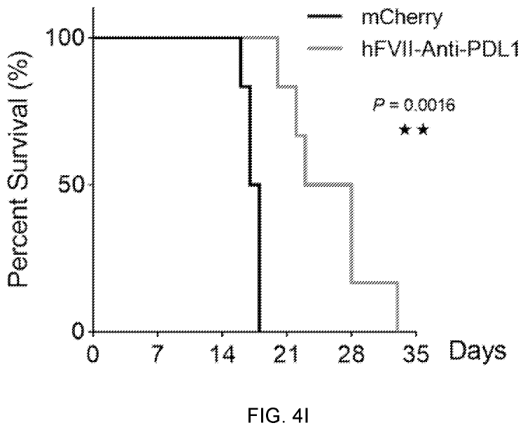

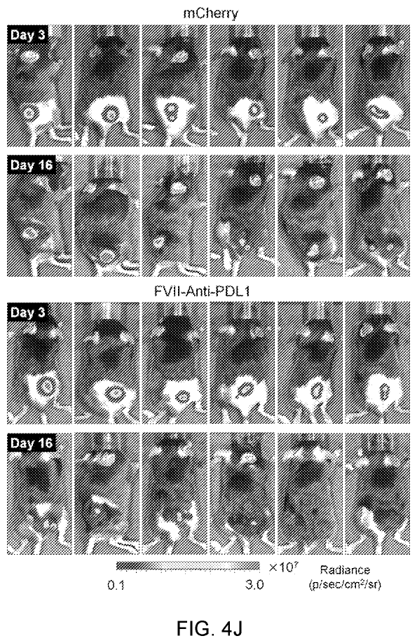

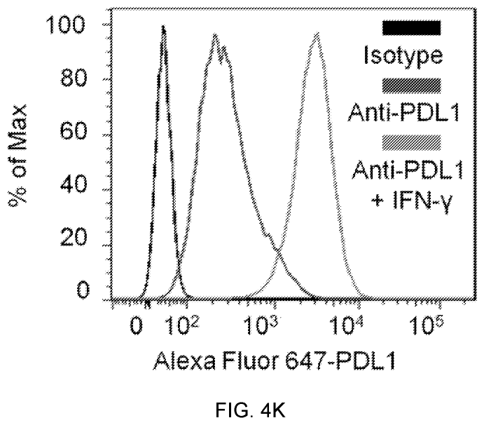

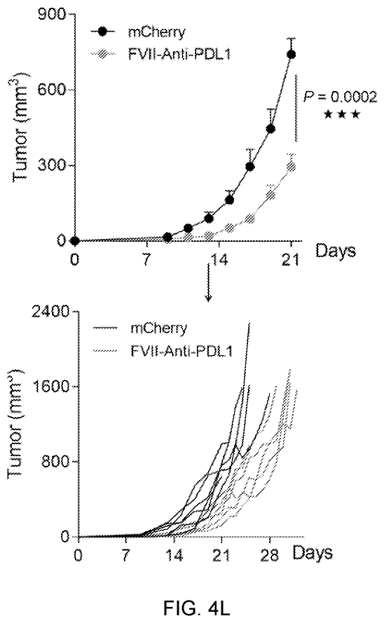

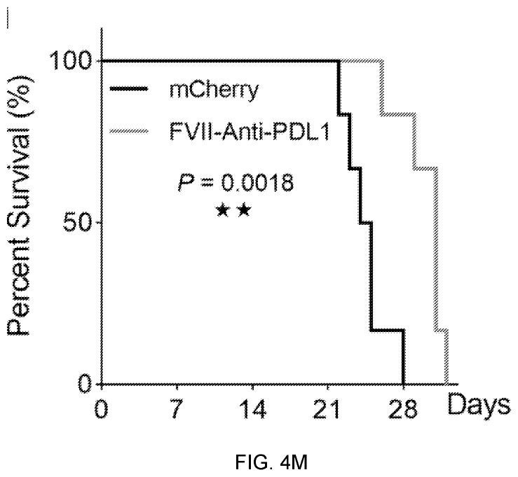

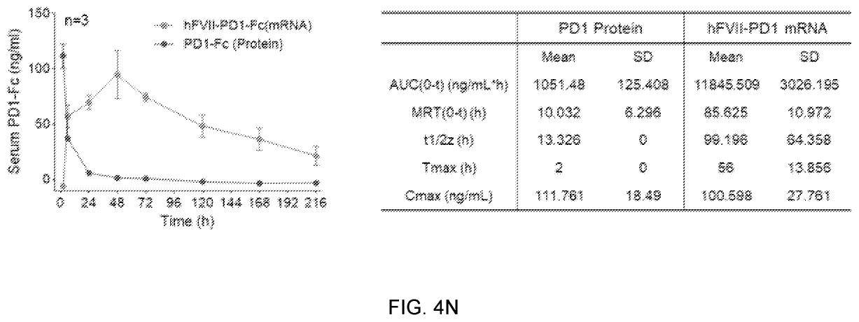

Next, use of the disclosed signal peptide were tested with anti-cancer polypeptides. For this two xenograft tumor models. MC38-Luc cells—an aggressive murine adenocarcinoma cell line containing a luciferase reporter construct, or B16F10-Luc cells—a murine melanoma cell line with a luciferase reporter construct, were injected subcutaneously into the right hind leg of C57BL6 mice and tumors were allowed to grow. To identify anti-cancer therapeutic potential of SP-mRNA LNPs, mice were first inoculated with tumor cells and then injected intravenously on days 3, 7, and 11 post-inoculation with mDLNPs containing either mCherry mRNA or mDLNPs encapsulating mRNA that encoded hFVII SP upstream from anti-PDL1 antibody (FIG. 4A-B).

A pharmacokinetic distribution of serum Anti-PDL1 levels was established with peak serum concentration occurring 48 h post IV injection—essentially mirroring the curve seen previously with hFVII Enbrel mRNA mDLNPs, and PDL1 expression on the surface of MC38 cells was determined using flow cytometry (FIG. 4C-D). Tumor growth was evaluated on days 3, 10, 24, and 32 via IVIS imaging for luminescence. In the group receiving mCherry mDLNPs, tumor development was rapid and a corresponding increase in luminescence, as well as volume at each time point was clearly evident. The tumor mass consumed the entire hind leg of each mouse by day 32. However, in the group treated with hFVII Anti-PDL1 mRNA mDLNPs, tumor growth noticeably declined by day 24, suggesting that treatment with hFVII Anti-PDL1 mRNA mDLNPs was effective at inhibiting tumor progression. As expected, this reduction was paralleled by a decrease in luminescence, significantly slower tumor development, and smaller tumor volume overall following resection on day 32 (FIG. 4E-H). Most notable was the extension in survival mediated by hFVII Anti-PDL1 mRNA mDLNPs. Mice in this treatment group lived nearly 2-fold longer than those in the control group (FIG. 4I). Similarly, mice inoculated with the B16F10-Luc tumors were injected via IV with mDLNPs containing mCherry mRNA or hFVII Anti-PDL1 mRNA. IVIS imaging at days 3 and 16 revealed a decline in tumor mass and luminescence, significantly slower rate of growth, and an extension in overall survival in the hFVII Anti-PDL1 mRNA group when compared with control (FIG. 4J-N).

### Example 5: Design of Novel Signal Peptide Sequences

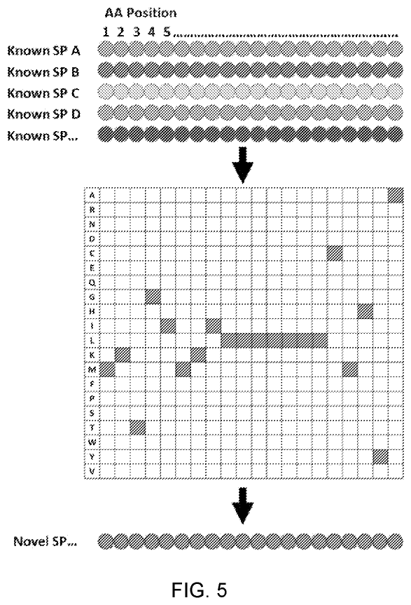

Engineering of novel signal peptide (SP) sequences coding for secretion into specific mRNA sequences was attempted to enable secretion of proteins into circulation that would ordinarily be confined to the intracellular space. To investigate this further, an exhaustive search for known SPs across multiple databases was conducted and combined with current findings into a single master database. From there, known SPs were categorized and a new list generated containing the amino acid sequences for 643 previously identified naturally existing SPs associated with secreted proteins. Due to the heterogeneity in amino acid sequence length, the list of 643 sequences was sorted by length wherein sequences of the same length were grouped accordingly (i.e., SP sequences containing 18 amino acids were all grouped together, SPs 19 amino acids in length were grouped together, etc.). Once the signal sequences were stratified by length, a matrix was created which allowed the identification of the frequency of each amino acid at its respective position along the peptide for all sequences of a particular length (see FIG. 5). After this the most frequent amino acid at each position were picked and strung them together thereby forming a novel amino acid sequence of a specified length. Through this process, 21 novel SP sequences were generated (see Table 2) varying in length from 15 amino acids to 35 amino acids that are not known to exist in nature (termed SP1-SP21).

TABLE 2
List of engineered sequences

| SEQ | | SEQ | |
|---|---|---|---|
| ID | | ID | |
| NOS: | Peptide | NOS: | DNA |
| 9 | MLLLLLLLLLAAVAA | 30 | ATGCTGCTGCTGCTGCTGCTGCTGCT |
| | | | GCTGGCGGCGGTGGCGGCG |
| 10 | MKVLLLLLFLLLLTLA | 31 | ATGAAAGTGCTGCTGCTGCTGCTGTT |
| | | | TCTGCTGCTGCTGACCCTGGCG |
| 11 | MWLLLALLLLLAGAAAA | 32 | ATGTGGCTGCTGCTGGCGCTGCTGC |
| | | | TGCTGCTGGCGGGCGCGGCGGCGG |
| | | | CG |
| 12 | MKLLLLLLLLLLLSGALA | 33 | ATGAAACTGCTGCTGCTGCTGCTGCT |
| | | | GCTGCTGCTGCTGAGCGGCGCGCTG |
| | | | GCG |
| 13 | MRLLLLLLLLLLLLASALA | 34 | ATGCGCCTGCTGCTGCTGCTGCTGCT |
| | | | GCTGCTGCTGCTGCTGGCGAGCGCG |
| | | | CTGGCG |
| 14 | MALLLLLLLLLLLLLALALA | 35 | ATGGCGCTGCTGCTGCTGCTGCTGC |
| | | | TGCTGCTGCTGCTGCTGCTGGCGCT |
| | | | GGCGCTGGCG |
| 15 | MALLALLLLLLLLLLLALAQA | 36 | ATGGCGCTGCTGGCGCTGCTGCTGC |
| | | | TGCTGCTGCTGCTGCTGCTGCTGGC |
| | | | GCTGGCGCAGGCG |
| 16 | MALLRLLLLLLLLLLLLAGVLG | 37 | ATGGCGCTGCTGCGCCTGCTGCTGC |
| | | | TGCTGCTGCTGCTGCTGCTGCTGCTG |
| | | | GCGGGCGTGCTGGGC |
| 17 | MALSFALLLLLLLLLLLSSCSLA | 38 | ATGGCGCTGAGCTTTGCGCTGCTGCT |
| | | | GCTGCTGCTGCTGCTGCTGCTGCTG |
| | | | AGCAGCTGCAGCCTGGCG |
| 18 | MARLALLLLLLLLLLLLLLPAVEA | 39 | ATGGCGCGCCTGGCGCTGCTGCTGC |
| | | | TGCTGCTGCTGCTGCTGCTGCTGCTG |
| | | | CTGCTGCCGGCGGTGGAAGCG |
| 19 | MGSLGLLLLLLLLLLLLLLLGGALA | 40 | ATGGGCAGCCTGGGCCTGCTGCTGC |
| | | | TGCTGCTGCTGCTGCTGCTGCTGCTG |
| | | | CTGCTGCTGGGCGGCGCGCTGGCG |
| 20 | MAAPLRSALLLLALLLLLLLLGEASA | 41 | ATGGCGGCGCCGCTGCGCAGCGCG |
| | | | CTGCTGCTGCTGGCGCTGCTGCTGC |
| | | | TGCTGCTGCTGCTGGGCGAAGCGAG |
| | | | CGCG |
| 21 | MALARLLLLLLLLLLLLLLLLLGGVLA | 42 | ATGGCGCTGGCGCGCCTGCTGCTGC |
| | | | TGCTGCTGCTGCTGCTGCTGCTGCTG |
| | | | CTGCTGCTGCTGCTGGGCGGCGTGC |
| | | | TGGCG |
| 22 | MAPGRTPLLALLLLLLLLLLLLPAAALA | 43 | ATGGCGCCGGGCCGCACCCCGCTGC |
| | | | TGGCGCTGCTGCTGCTGCTGCTGCT |
| | | | GCTGCTGCTGCTGCTGCCGGCGGCG |
| | | | GCGCTGGCG |
| 23 | MPARGLRALLLLLLLLLLLLLLPLRPAQA | 44 | ATGCCGGCGCGCGGCCTGCGCGCG |
| | | | CTGCTGCTGCTGCTGCTGCTGCTGCT |
| | | | GCTGCTGCTGCTGCTGCCGCTGCGC |
| | | | CCGGCGCAGGCG |
| 24 | MAAARSRGARWLKLLLLLLLLLLLPPGALA | 45 | ATGGCGGCGGCGCGCAGCCGCGGC |
| | | | GCGCGCTGGCTGAAACTGCTGCTGC |
| | | | TGCTGCTGCTGCTGCTGCTGCTGCC |
| | | | GCCGGGCGCGCTGGCG |
| 25 | MALASRGSAALALLALLLLLLLALLLAGAAA | 46 | ATGGCGCTGGCGAGCCGCGGCAGC |
| | | | GCGGCGCTGGCGCTGCTGGCGCTGC |
| | | | TGCTGCTGCTGCTGCTGGCGCTGCT |
| | | | GCTGGCGGGCGCGGCGGCG |
| 26 | MRAGPALAVRAALRALLLLLLLGLLLLDGSDA | 47 | ATGCGCGCGGGCCCGGCGCTGGCG |
| | | | GTGCGCGCGGCGCTGCGCGCGCTG |
| | | | CTGCTGCTGCTGCTGCTGGGCCTGC |
| | | | TGCTGCTGGATGGCAGCGATGCG |
| 27 | MAALAPKVSPAAAGVLLLLLLLLLLLLPGGAAA | 48 | ATGGCGGCGCTGGCGCCGAAAGTGA |
| | | | GCCCGGCGGCGGCGGGCGTGCTGC |
| | | | TGCTGCTGCTGCTGCTGCTGCTGCTG |
| | | | CTGCTGCCGGGCGGCGCGGCGGCG |
| 28 | MARATLSAAPSNPRLLRVALLLLLLLAALRRAAG | 49 | ATGGCGCGCGCGACCCTGAGCGCGG |
| | | | CGCCGAGCAACCCGCGCCTGCTGCG |
| | | | CGTGGCGCTGCTGCTGCTGCTGCTG |
| | | | CTGGCGGCGCTGCGCCGCGCGGCG |
| | | | GGC |
| 29 | MAGWRRMAKALPAPAGGPACALLLLLLLLAAGAQA | 50 | ATGGCGGGCTGGCGCCGCATGGCGA |
| | | | AAGCGCTGCCGGCGCCGGGGGCG |
| | | | GCCCGGCGTGCGCGCTGCTGCTGCT |
| | | | GCTGCTGCTGCTGGCGGCGGGCGC |
| | | | GCAGGCG |

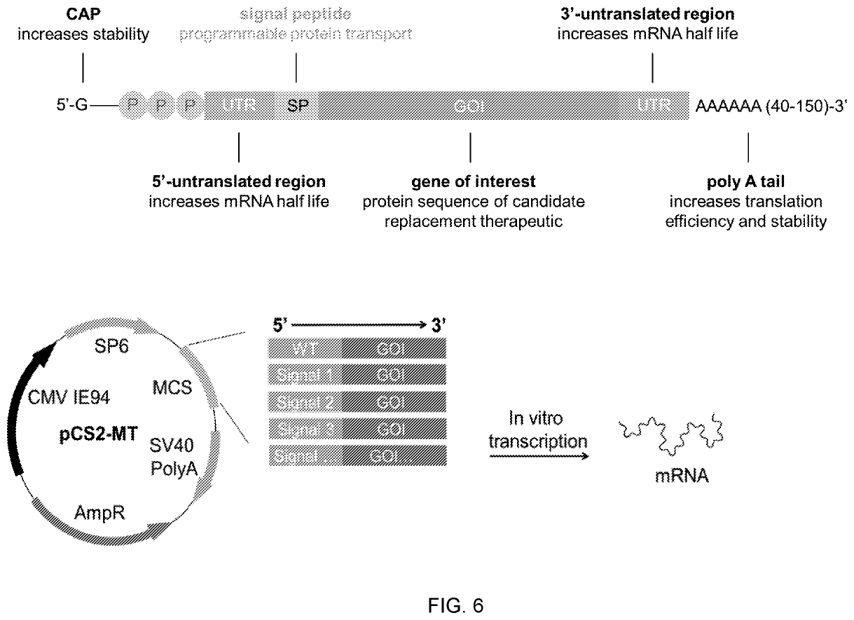

In order to determine whether the 21 novel SP sequences were able to induce secretion, the sequences were incorporated upstream from an amino acid sequence encoding human erythropoietin (SEQ ID NOS. 58: hEPO without its natural signal peptide). However, hEPO is known to contain a SP sequence of its own a pDNA backbone containing hEPO that lacks its endogenous SP (NF-NSP-hEPO) was created. Using a reverse codon generator tool, nucleic acid sequences were synthesized for each of the 21 peptides and subsequently the corresponding oligonucleotides were ordered. Through a series of cloning and PCR reactions, pDNA vectors containing each of the 21 SPs directly upstream from NF-NSP-hEPO were constructed (FIG. 6). To ensure that novel SPs were responsible for any secretion that may occur, a pDNA backbone containing a functional hEPO without its SP was constructed as control. However, the kozak sequence for hEPO is located upstream from hEPO's endogenous SP which was previously removed. Therefore, a kozak sequence was inserted (GCCACCATG) upstream from the SP-truncated hEPO sequence which allowed hEPO mRNA to be translated but removes its secretion capability (NSP-hEPO). With the pDNA backbones in hand, in-vitro transcription (IVT) reactions were performed for each of the novel SP-hEPO sequences as well as the NSP-hEPO sequence to generate mRNA containing an optimized 5′ UTR (SEQ ID NO: 59), kozak sequence, novel signal peptide sequence, hEPO protein sequence, 3′ UTR (SEQ ID NO: 60), and finally an optimized poly-A tail (SEQ ID NO. 61)

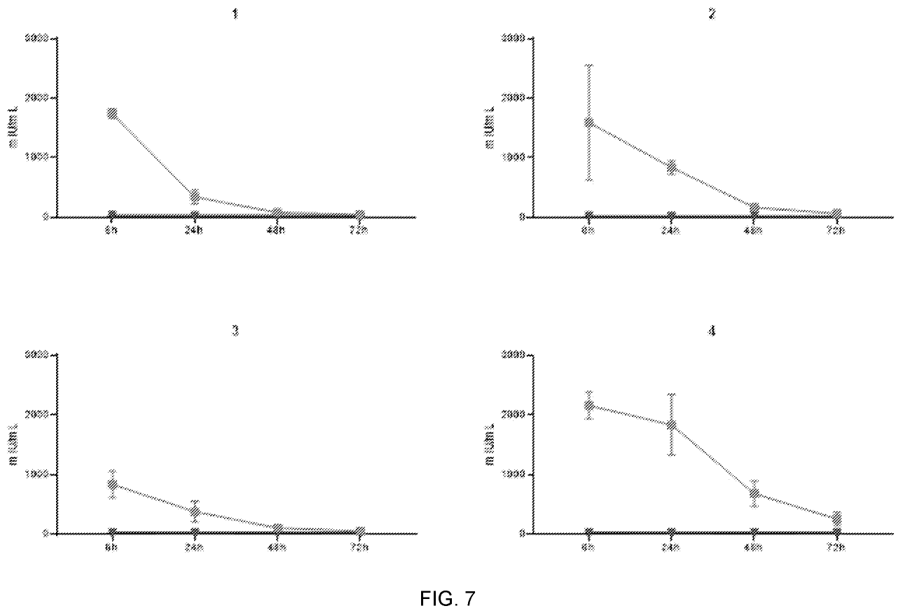

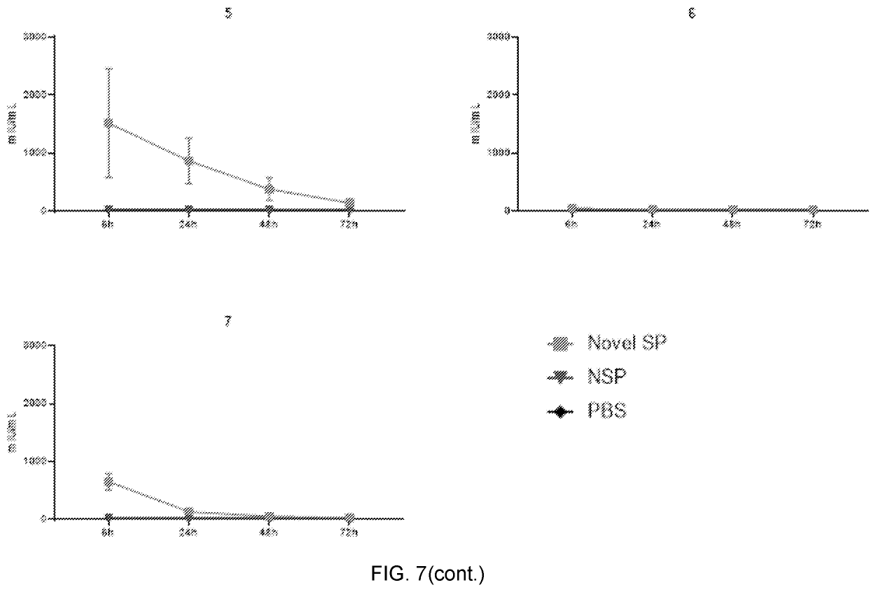

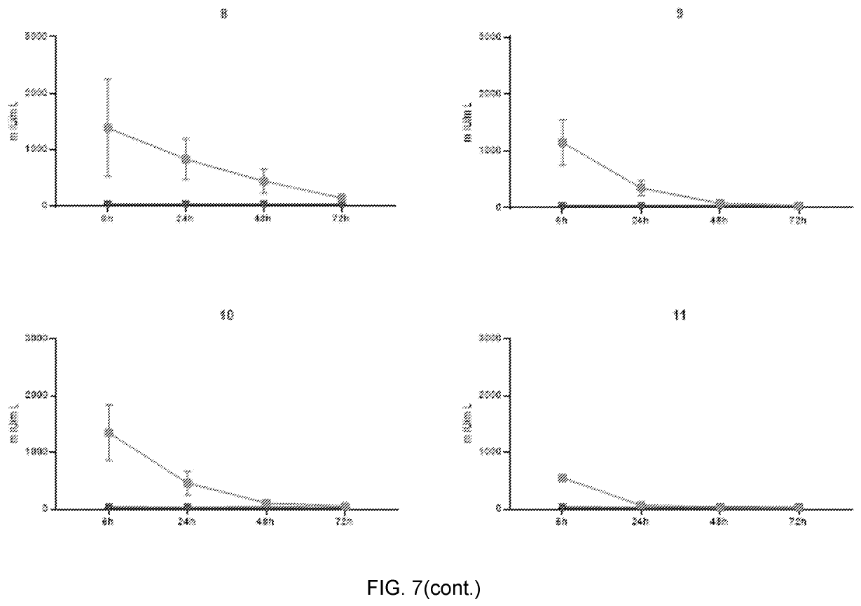

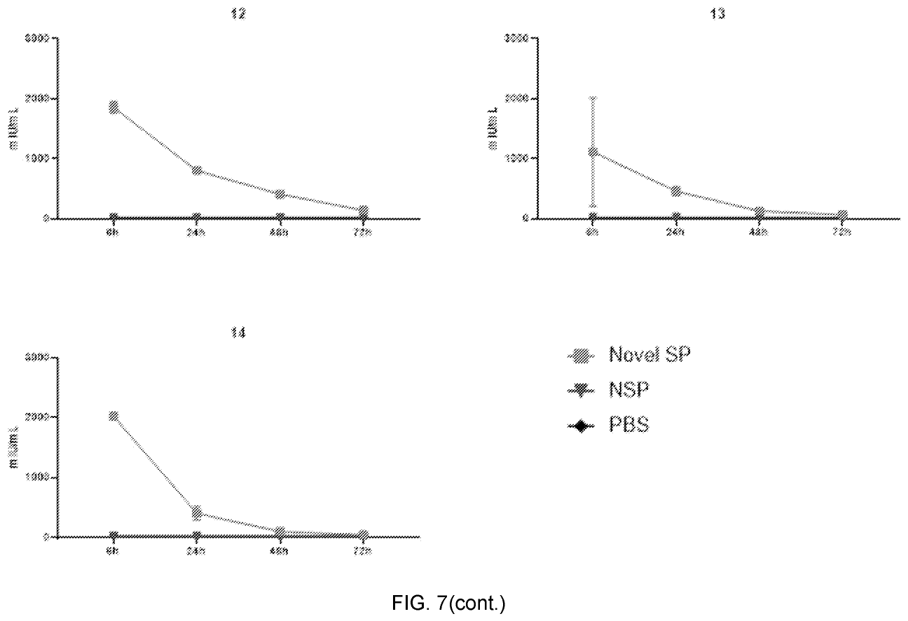

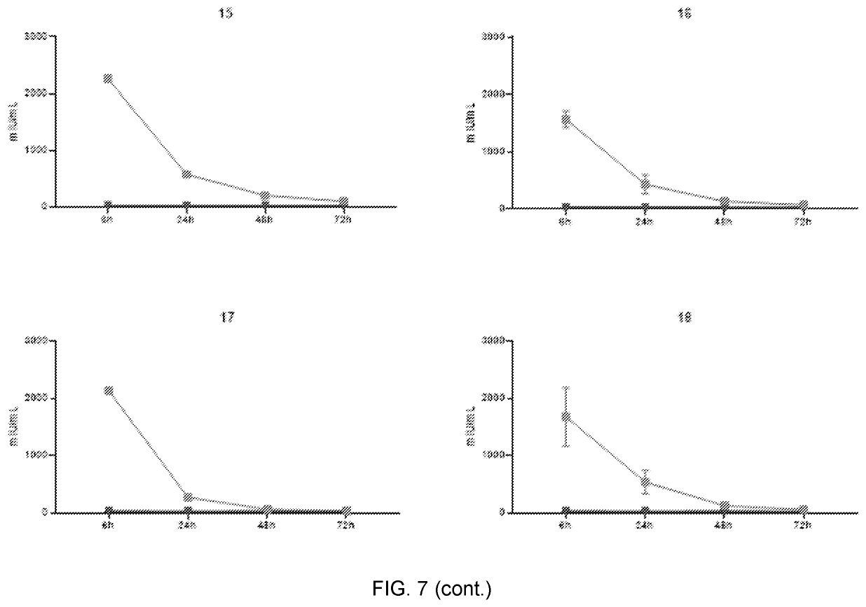

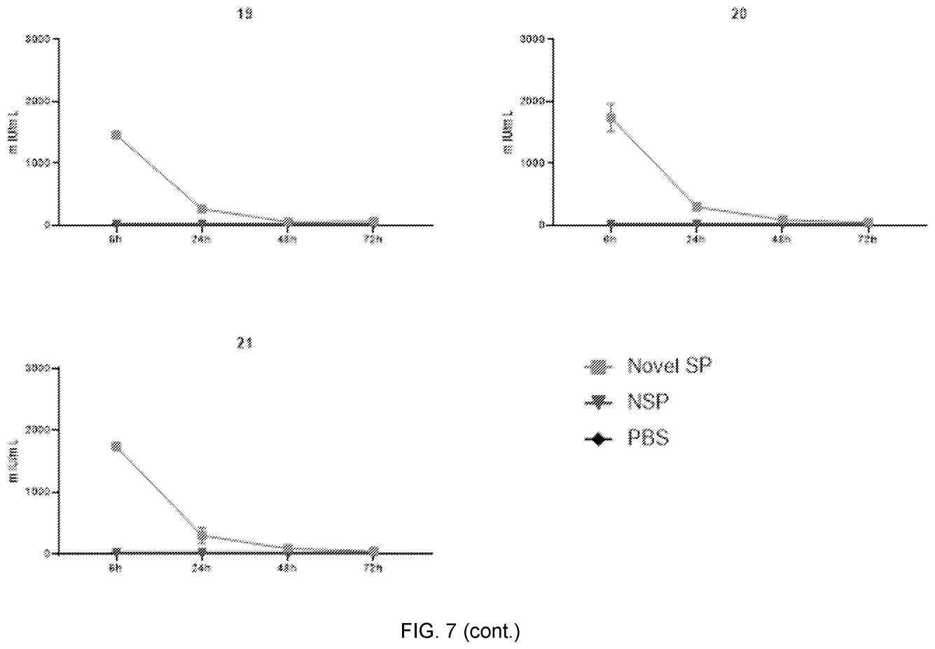

The newly synthesized mRNAs were then encapsulated into iPhos LNPs (9A1-P9/Cholesterol/DODAP/DMG-PEG, 25:30:30:1 mol/mol; 18:1 9A1-P9: nucleic acid, wt/wt) and administered intravenously into mice at a dosage of 0.5 mg/kg. Blood was collected from mice at 6, 24, 48, and 72 h for each construct and analyzed via hEPO enzyme-linked immunosorbent assay (ELISA) to determine serum hEPO concentration mlU/mL (FIG. 7). Successful secretion of SP-hEPO with enhanced differential kinetic profiles (Cₘₐₓ, t_{1/2} and area under the curve (AUC)) with respect to NSP-hEPO control could be seen.

### SEQUENCES:

| SEQ | | | |
|---|---|---|---|
| ID | | | |
| NOS | Name | Type | Protein sequence |
| 1 | hAlb-SP | Protein | MKWVTFISLLFLFSSAYS |
| 2 | hApoB- | Protein | MDPPRPALLALLALPALLLLLLAGARA |
| | SP | | |
| 3 | gLuc-SP | Protein | MGVKVLFALICIAVAEA |
| 4 | hFVII-SP | Protein | MVSQALRLLCLLLGLQGCLA |
| 5 | hAlb-SP | DNA | ATGAAGTGGGTAACCTTTATTTCCCTTCTTTTTCTCTTTAG |
| | | | CTCGGCTTATTCCAGGGGTGTGTTTCGTCGA |
| 6 | hApoB- | DNA | ATGGACCCGCCGAGGCCCGCGCTGCTGGCGCTGCTGGC |
| | SP | | GCTGCCTGCGCTGCTGCTGCTGCTGCTGGCGGGCGCCA |
| | | | GGGCC |
| 7 | gLuc-SP | DNA | ATGGGCGTGAAAGTGCTGTTTGCGCTGATTTGCATTGCG |
| | | | GTGGCGGAAGCG |
| 8 | hFVII-SP | DNA | ATGGTCTCCCAGGCCCTCAGGCTCCTCTGCCTTCTGCTT |
| | | | GGGCTTCAGGGCTGCCTGGCT |
| 9 | | Protein | MLLLLLLLLLAAVAA |
| 10 | | Protein | MKVLLLLLFLLLLTLA |
| 11 | | Protein | MWLLLALLLLLAGAAAA |
| 12 | | Protein | MKLLLLLLLLLLLSGALA |
| 13 | | Protein | MRLLLLLLLLLLLLASALA |
| 14 | | Protein | MALLLLLLLLLLLLLALALA |
| 15 | | Protein | MALLALLLLLLLLLLLALAQA |
| 16 | | Protein | MALLRLLLLLLLLLLLLAGVLG |
| 17 | | Protein | MALSFALLLLLLLLLLLSSCSLA |
| 18 | | Protein | MARLALLLLLLLLLLLLLLPAVEA |
| 19 | | Protein | MGSLGLLLLLLLLLLLLLLLGGALA |
| 20 | | Protein | MAAPLRSALLLLALLLLLLLLGEASA |
| 21 | | Protein | MALARLLLLLLLLLLLLLLLLLGGVLA |
| 22 | | Protein | MAPGRTPLLALLLLLLLLLLLLPAAALA |
| 23 | | Protein | MPARGLRALLLLLLLLLLLLLLPLRPAQA |
| 24 | | Protein | MAAARSRGARWLKLLLLLLLLLLLPPGALA |
| 25 | | Protein | MALASRGSAALALLALLLLLLLALLLAGAAA |
| 26 | | Protein | MRAGPALAVRAALRALLLLLLLGLLLLDGSDA |
| 27 | | Protein | MAALAPKVSPAAAGVLLLLLLLLLLLLPGGAAA |
| 28 | | Protein | MARATLSAAPSNPRLLRVALLLLLLLAALRRAAG |
| 29 | | Protein | MAGWRRMAKALPAPAGGPACALLLLLLLLAAGAQA |
| 30 | | DNA | ATGCTGCTGCTGCTGCTGCTGCTGCTGCTGGCGGCGGT |
| | | | GGCGGCG |
| 31 | | DNA | ATGAAAGTGCTGCTGCTGCTGCTGTTTCTGCTGCTGCTG |
| | | | ACCCTGGCG |
| 32 | | DNA | ATGTGGCTGCTGCTGGCGCTGCTGCTGCTGCTGGCGGG |
| | | | CGCGGCGGCGGCG |
| 33 | | DNA | ATGAAACTGCTGCTGCTGCTGCTGCTGCTGCTGCTGCTG |
| | | | AGCGGCGCGCTGGCG |
| 34 | | DNA | ATGCGCCTGCTGCTGCTGCTGCTGCTGCTGCTGCTGCTG |
| | | | CTGGCGAGCGCGCTGGCG |
| 35 | | DNA | ATGGCGCTGCTGCTGCTGCTGCTGCTGCTGCTGCTGCTG |
| | | | CTGCTGGCGCTGGCGCTGGCG |
| 36 | | DNA | ATGGCGCTGCTGGCGCTGCTGCTGCTGCTGCTGCTGCT |
| | | | GCTGCTGCTGGCGCTGGCGCAGGCG |
| 37 | | DNA | ATGGCGCTGCTGCGCCTGCTGCTGCTGCTGCTGCTGCT |
| | | | GCTGCTGCTGCTGGCGGGCGTGCTGGGC |
| 38 | | DNA | ATGGCGCTGAGCTTTGCGCTGCTGCTGCTGCTGCTGCTG |
| | | | CTGCTGCTGCTGAGCAGCTGCAGCCTGGCG |
| 39 | | DNA | ATGGCGCGCCTGGCGCTGCTGCTGCTGCTGCTGCTGCT |
| | | | GCTGCTGCTGCTGCTGCTGCCGGCGGTGGAAGCG |
| 40 | | DNA | ATGGGCAGCCTGGGCCTGCTGCTGCTGCTGCTGCTGCT |
| | | | GCTGCTGCTGCTGCTGCTGCTGGGCGGCGCGCTGGCG |
| 41 | | DNA | ATGGCGGCGCCGCTGCGCAGCGCGCTGCTGCTGCTGGC |
| | | | GCTGCTGCTGCTGCTGCTGCTGCTGGGCGAAGCGAGCG |
| | | | CG |
| 42 | | DNA | ATGGCGCTGGCGCGCCTGCTGCTGCTGCTGCTGCTGCT |
| | | | GCTGCTGCTGCTGCTGCTGCTGCTGCTGGGCGGCGTGC |
| | | | TGGCG |
| 43 | | DNA | ATGGCGCCGGGCCGCACCCCGCTGCTGGCGCTGCTGCT |
| | | | GCTGCTGCTGCTGCTGCTGCTGCTGCTGCCGGCGGCGG |
| | | | CGCTGGCG |
| 44 | | DNA | ATGCCGGCGCGCGGCCTGCGCGCGCTGCTGCTGCTGCT |
| | | | GCTGCTGCTGCTGCTGCTGCTGCTGCTGCCGCTGCGCC |
| | | | CGGCGCAGGCG |
| 45 | | DNA | ATGGCGGCGGCGCGCAGCCGCGGCGCGCGCTGGCTGA |
| | | | AACTGCTGCTGCTGCTGCTGCTGCTGCTGCTGCTGCCGC |
| | | | CGGGCGCGCTGGCG |
| 46 | | DNA | ATGGCGCTGGCGAGCCGCGGCAGCGCGGCGCTGGCGC |
| | | | TGCTGGCGCTGCTGCTGCTGCTGCTGCTGGCGCTGCTG |
| | | | CTGGCGGGCGCGGCGGCG |
| 47 | | DNA | ATGCGCGCGGGCCCGGCGCTGGCGGTGCGCGCGGCGC |
| | | | TGCGCGCGCTGCTGCTGCTGCTGCTGCTGGGCCTGCTG |
| | | | CTGCTGGATGGCAGCGATGCG |
| 48 | | DNA | ATGGCGGCGCTGGCGCCGAAAGTGAGCCCGGCGGCGG |
| | | | CGGGCGTGCTGCTGCTGCTGCTGCTGCTGCTGCTGCTG |
| | | | CTGCTGCCGGGCGGCGCGGCGGCG |
| 49 | | DNA | ATGGCGCGCGCGACCCTGAGCGCGGCGCCGAGCAACC |
| | | | CGCGCCTGCTGCGCGTGGCGCTGCTGCTGCTGCTGCTG |
| | | | CTGGCGGCGCTGCGCCGCGCGGCGGGC |
| 50 | | DNA | ATGGCGGGCTGGCGCCGCATGGCGAAAGCGCTGCCGG |
| | | | CGCCGGCGGGCGGCCCGGCGTGCGCGCTGCTGCTGCT |
| | | | GCTGCTGCTGCTGGCGGCGGGCGCGCAGGCG |
| 51 | mCherry | DNA | ATGGTGAGCAAGGGCGAGGAGGATAACATGGCCATCATC |
| | | | AAGGAGTTCATGCGCTTCAAGGTGCACATGGAGGGCTCC |
| | | | GTGAACGGCCACGAGTTCGAGATCGAGGGCGAGGGCGA |
| | | | GGGCCGCCCCTACGAGGGCACCCAGACCGCCAAGCTGA |
| | | | AGGTGACCAAGGGTGGCCCCCTGCCCTTCGCCTGGGAC |
| | | | ATCCTGTCCCCTCAGTTCATGTACGGCTCCAAGGCCTAC |
| | | | GTGAAGCACCCCGCCGACATCCCCGACTACTTGAAGCTG |
| | | | TCCTTCCCCGAGGGCTTCAATTGGGAGCGCGTGATGAAC |
| | | | TTCGAGGACGGCGGCGTGGTGACCGTGACCCAGGACTC |
| | | | CTCCCTGCAGGACGGCGAGTTCATCTACAAGGTGAAGCT |
| | | | GCGCGGCACCAACTTCCCCTCCGACGGCCCCGTAATGC |
| | | | AGTGTCGTACCATGGGCTGGGAGGCCTCCACTGAGCGG |
| | | | ATGTACCCCGAGGACGGCGCCCTGAAGGGCGAGATCAA |
| | | | GCAGAGGCTGAAGCTGAAGGACGGCGGCCACTACGACG |
| | | | CTGAGGTCAAGACCACCTACAAGGCCAAGAAGCCCGTGC |
| | | | AGCTGCCCGGCGCCTACAACGTCGACATCAAGTTGGACA |
| | | | TCCTTTCCCACAACGAGGACTACACCATCGTGGAACAGT |
| | | | ACGAACGCGCCGAGGGCCGCCACTCCACCGGCGGCATG |
| | | | GACGAGCTGTACAAGTAA |
| 52 | Enbrel | DNA | TTGCCCGCCCAGGTGGCATTTACACCCTACGCCCCGGAG |
| | | | CCCGGGAGCACATGCCGGCTCAGAGAATACTATGACCAG |
| | | | ACAGCTCAGATGTGCTGCAGCAAATGCTCGCCGGGCCAA |
| | | | CATGCAAAAGTCTTCTGTACCAAGACCTCGGACACCGTG |
| | | | TGTGACTCCTGTGAGGACAGCACATACACCCAGCTCTGG |
| | | | AACTGGGTTCCCGAGTGCTTGAGCTGTGGCTCCCGCTGT |
| | | | AGCTCTGACCAGGTGGAAACTCAAGCCTGCACTCGGGAA |
| | | | CAGAACCGCATCTGCACCTGCAGGCCCGGCTGGTACTG |
| | | | CGCGCTGAGCAAGCAGGAGGGGTGCCGGCTGTGCGCG |
| | | | CCGCTGCGCAAGTGCCGCCCGGGCTTCGGCGTGGCCAG |
| | | | ACCAGGAACTGAAACATCAGACGTGGTGTGCAAGCCCTG |
| | | | TGCCCCGGGGACGTTCTCCAACACGACTTCATCCACGGA |
| | | | TATTTGCAGGCCCCACCAGATCTGTAACGTGGTGGCCAT |
| | | | CCCTGGGAATGCAAGCATGGATGCAGTCTGCACGTCCAC |
| | | | GTCCCCCACCCGGAGTATGGCCCCAGGGGCAGTACACT |
| | | | TACCCCAGCCAGTGTCCACACGATCCCAACACACGCAGC |
| | | | CAACTCCAGAACCCAGCACTGCTCCAAGCACCTCCTTCC |
| | | | TGCTCCCAATGGGCCCCAGCCCCCCAGCTGAAGGGAGC |
| | | | ACTGGCGACGAGCCCAAATCTTGTGACAAAACTCACACA |
| | | | TGCCCACCGTGCCCAGCACCTGAACTCCTGGGGGGACC |
| | | | GTCAGTCTTCCTCTTCCCCCCAAAACCCAAGGACACCCT |
| | | | CATGATCTCCCGGACCCCTGAGGTCACATGCGTGGTGGT |
| | | | GGACGTGAGCCACGAAGACCCTGAGGTCAAGTTCAACTG |
| | | | GTACGTGGACGGCGTGGAGGTGCATAATGCCAAGACAAA |
| | | | GCCGCGGGAGGAGCAGTACAACAGCACGTACCGTGTGG |
| | | | TCAGCGTCCTCACCGTCCTGCACCAGGACTGGCTGAATG |
| | | | GCAAGGAGTACAAGTGCAAGGTCTCCAACAAAGCCCTCC |
| | | | CAGCCCCCATCGAGAAAACCATCTCCAAAGCCAAAGGGC |
| | | | AGCCCCGAGAACCACAGGTGTACACCCTGCCCCCATCCC |
| | | | GGGAGGAGATGACCAAGAACCAGGTCAGCCTGACCTGC |
| | | | CTGGTCAAAGGCTTCTATCCCAGCGACATCGCCGTGGAG |
| | | | TGGGAGAGCAATGGGCAGCCGGAGAACAACTACAAGAC |
| | | | CACGCCTCCCGTGCTGGACTCCGACGGCTCCTTCTTCCT |
| | | | CTATAGCAAGCTCACCGTGGACAAGAGCAGGTGGCAGCA |
| | | | GGGGAACGTCTTCTCATGCTCCGTGATGCATGAGGCTCT |
| | | | GCACAACCACTACACGCAGAAGAGCCTCTCCCTGTCCCC |
| | | | GGGTAAATGA |
| 53 | PD-L1 | DNA | GAGGTCCAGTTGGTAGAGTCTGGGGGGGGCCTCGTGCA |
| | | | GCCCGGTGGAAGCCTTCGCCTGAGTTGTGCAGCCAGTG |
| | | | GCTTCACCTTCTCTGATAGCTGGATTCATTGGGTAAGGCA |
| | | | GGCACCCGGTAAAGGTCTCGAGTGGGTTGCATGGATTTC |
| | | | CCCTTACGGGGGGAGCACTTACTATGCTGATTCCGTTAA |
| | | | AGGCCGCTTTACAATATCTGCAGATACCTCTAAAAACACT |
| | | | GCATACTTGCAAATGAACTCACTGCGAGCTGAAGACACT |
| | | | GCCGTATATTACTGCGCAAGAAGGCACTGGCCCGGTGG |
| | | | CTTTGATTATTGGGGACAAGGAACCCTTGTAACTGTATCT |
| | | | GGCGGGGGAGGAAGCGGTGGAGGTGGGTCAGGCGGTG |
| | | | GAGGTAGCGGAGGGGGTGGCTCTGATATACAGATGACA |
| | | | CAGTCACCTAGTAGCCTGTCTGCTAGTGTTGGGGACCGA |
| | | | GTCACTATAACATGTAGAGCTTCTCAGGATGTCTCAACCG |
| | | | CAGTAGCCTGGTACCAGCAAAAACCTGGTAAGGCCCCCA |
| | | | AACTCCTCATATACTCTGCTTCATTTCTCTATTCTGGTGTG |
| | | | CCAAGCAGATTCAGTGGTAGCGGCAGTGGCACCGACTTC |
| | | | ACCCTGACCATATCTTCCTTGCAGCCTGAGGACTTTGCAA |
| | | | CATACTACTGTCAGCAGTACCTCTATCATCCTGCAACATT |
| | | | CGGACAAGGTACCAAAGTAGAGATCAAAGAGCCCAAATC |
| | | | TTGTGACAAAACTCACACATGCCCACCGTGCCCAGCACC |
| | | | TGAACTCCTGGGGGGACCGTCAGTCTTCCTCTTCCCCCC |
| | | | AAAACCCAAGGACACCCTCATGATCTCCCGGACCCCTGA |
| | | | GGTCACATGCGTGGTGGTGGACGTGAGCCACGAAGACC |
| | | | CTGAGGTCAAGTTCAACTGGTACGTGGACGGCGTGGAG |
| | | | GTGCATAATGCCAAGACAAAGCCGCGGGAGGAGCAGTA |
| | | | CAACAGCACGTACCGTGTGGTCAGCGTCCTCACCGTCCT |
| | | | GCACCAGGACTGGCTGAATGGCAAGGAGTACAAGTGCAA |
| | | | GGTCTCCAACAAAGCCCTCCCAGCCCCCATCGAGAAAAC |
| | | | CATCTCCAAAGCCAAAGGGCAGCCCCGAGAACCACAGGT |
| | | | GTACACCCTGCCCCCATCCCGGGATGAGCTGACCAAGAA |
| | | | CCAGGTCAGCCTGACCTGCCTGGTCAAAGGCTTCTATCC |
| | | | CAGCGACATCGCCGTGGAGTGGGAGAGCAATGGGCAGC |
| | | | CGGAGAACAACTACAAGACCACGCCTCCCGTGCTGGACT |
| | | | CCGACGGCTCCTTCTTCCTCTACAGCAAGCTCACCGTGG |
| | | | ACAAGAGCAGGTGGCAGCAGGGGAACGTCTTCTCATGCT |
| | | | CCGTGATGCATGAGGCTCTGCACAACCACTACACGCAGA |
| | | | AAAGCCTCTCCCTGTCCCCGGGTAAAGGCGGCTCTCACC |
| | | | ACCACCATCACCACTGA |
| 54 | hEPO | DNA | GCCCCCCCCCGGCTGATCTGCGACAGCCGGGTGCTGGA |
| | | | GCGGTACCTGCTGGAGGCCAAGGAGGCCGAGAACATCA |
| | | | CCACCGGCTGCGCCGAGCACTGCAGCCTGAACGAGAAC |
| | | | ATCACCGTGCCCGACACCAAGGTGAACTTCTACGCCTGG |
| | | | AAGCGGATGGAGGTGGGCCAGCAGGCCGTGGAGGTGTG |
| | | | GCAGGGCCTGGCCCTGCTGAGCGAGGCCGTGCTGCGG |
| | | | GGCCAGGCCCTGCTGGTGAACAGCAGCCAGCCCTGGGA |
| | | | GCCCCTGCAGCTGCACGTGGACAAGGCCGTGAGCGGCC |
| | | | TGCGGAGCCTGACCACCCTGCTGCGGGCCCTGGGCGCC |
| | | | CAGAAGGAGGCCATCAGCCCCCCCGACGCCGCCAGCGC |
| | | | CGCCCCCCTGCGGACCATCACCGCCGACACCTTCCGGA |
| | | | AGCTGTTCCGGGTGTACAGCAACTTCCTGCGGGGCAAGC |
| | | | TGAAGCTGTACACCGGCGAGGCCTGCCGGACCGGCGAC |
| | | | CGGTGA |
| 55 | mCherry | Protein | MVSKGEEDNMAIIKEFMRFKVHMEGSVNGHEFEIEGEGEG |
| | | | RPYEGTQTAKLKVTKGGPLPFAWDILSPQFMYGSKAYVKHP |
| | | | ADIPDYLKLSFPEGFNWERVMNFEDGGVVTVTQDSSLQDG |
| | | | EFIYKVKLRGTNFPSDGPVMQCRTMGWEASTERMYPEDGA |
| | | | LKGEIKQRLKLKDGGHYDAEVKTTYKAKKPVQLPGAYNVDI |
| | | | KLDILSHNEDYTIVEQYERAEGRHSTGGMDELYK |
| 56 | Enbrel | Protein | LPAQVAFTPYAPEPGSTCRLREYYDQTAQMCCSKCSPGQH |
| | | | AKVFCTKTSDTVCDSCEDSTYTQLWNWVPECLSCGSRCSS |
| | | | DQVETQACTREQNRICTCRPGWYCALSKQEGCRLCAPLRK |
| | | | CRPGFGVARPGTETSDVVCKPCAPGTFSNTTSSTDICRPH |
| | | | QICNVVAIPGNASMDAVCTSTSPTRSMAPGAVHLPQPVSTR |
| | | | SQHTQPTPEPSTAPSTSFLLPMGPSPPAEGSTGDEPKSCD |
| | | | KTHTCPPCPAPELLGGPSVFLFPPKPKDTLMISRTPEVTCVV |
| | | | VDVSHEDPEVKFNWYVDGVEVHNAKTKPREEQYNSTYRVV |
| | | | SVLTVLHQDWLNGKEYKCKVSNKALPAPIEKTISKAKGQPR |
| | | | EPQVYTLPPSREEMTKNQVSLTCLVKGFYPSDIAVEWESNG |
| | | | QPENNYKTTPPVLDSDGSFFLYSKLTVDKSRWQQGNVFSC |
| | | | SVMHEALHNHYTQKSLSLSPGK |
| 57 | PD-L1 | Protein | EVOLVESGGGLVQPGGSLRLSCAASGFTFSDSWIHWVRQA |
| | | | PGKGLEWVAWISPYGGSTYYADSVKGRFTISADTSKNTAYL |
| | | | QMNSLRAEDTAVYYCARRHWPGGFDYWGQGTLVTVSGG |
| | | | GGSGGGGSGGGGSGGGGSDIQMTQSPSSLSASVGDRVTI |
| | | | TCRASQDVSTAVAWYQQKPGKAPKLLIYSASFLYSGVPSRF |
| | | | SGSGSGTDFTLTISSLQPEDFATYYCQQYLYHPATFGQGTK |
| | | | VEIKEPKSCDKTHTCPPCPAPELLGGPSVFLFPPKPKDTLMI |
| | | | SRTPEVTCVVVDVSHEDPEVKFNWYVDGVEVHNAKTKPRE |
| | | | EQYNSTYRVVSVLTVLHQDWLNGKEYKCKVSNKALPAPIEK |
| | | | TISKAKGQPREPQVYTLPPSRDELTKNQVSLTCLVKGFYPS |
| | | | DIAVEWESNGQPENNYKTTPPVLDSDGSFFLYSKLTVDKSR |
| | | | WQQGNVFSCSVMHEALHNHYTQKSLSLSPGKGGSHHHHH |
| | | | H |
| 58 | hEPO | Protein | APPRLICDSRVLERYLLEAKEAENITTGCAEHCSLNENITVPD |
| | | (no signal | TKVNFYAWKRMEVGQQAVEVWQGLALLSEAVLRGQALLVN |
| | | peptide) | SSQPWEPLQLHVDKAVSGLRSLTTLLRALGAQKEAISPPDA |
| | | | ASAAPLRTITADTFRKLFRVYSNFLRGKLKLYTGEACRTGDR |
| 59 | 5' UTR | DNA | ACATTTGCTTCTGACACAACTGTGTTCACTAGCAACCTCA |
| | | | AACAGACACC |
| 60 | 3'UTR | DNA | GCTCGCTTTCTTGCTGTCCAATTTCTATTAAAGGTTCCTTT |
| | | | GTTCCCTAAGTCCAACTACTAAACTGGGGGATATTATGAA |
| | | | GGGCCTTGAGCATCTGGATTCTGCCTAATAAAAAACATTT |
| | | | ATTTTCATTGC |
| 61 | PolyA | DNA | AAAAAAAAAAAAAAAAAAAAAAAAAAAAAAAAAAAAAAAAA |
| | | | AAAAAAAAAAAAAAAAAAAAAAAAAAAAAAAAAAAAAAAAA |
| | | | AAAAAAAAAAAAAAAAAAAAAAAAAAAAAAAAAAA |
| 62 | NC- | Protein | MEDAKNIKKGPAPFYPLEDGMVSKGEEDNMAIIKEFMRFKV |
| | mCherry | | HMEGSVNGHEFEIEGEGEGRPYEGTQTAKLKVTKGGPLPF |
| | (luciferase- | | AWDILSPQFMYGSKAYVKHPADIPDYLKLSFPEGFNWERVM |
| | mCherry) | | NFEDGGVVTVTQDSSLQDGEFIYKVKLRGTNFPSDGPVMQ |
| | | | CRTMGWEASTERMYPEDGALKGEIKQRLKLKDGGHYDAEV |
| | | | KTTYKAKKPVQLPGAYNVDIKLDILSHNEDYTIVEQYERAEG |
| | | | RHSTGGMDELYK |
| 63 | hAlb- | Protein | MKWVTFISLLFLFSSAYSMVSKGEEDNMAIIKEFMRFKVHME |
| | mCherry | | GSVNGHEFEIEGEGEGRPYEGTQTAKLKVTKGGPLPFAWDI |
| | | | LSPQFMYGSKAYVKHPADIPDYLKLSFPEGFNWERVMNFE |
| | | | DGGVVTVTQDSSLQDGEFIYKVKLRGTNFPSDGPVMQCRT |
| | | | MGWEASTERMYPEDGALKGEIKQRLKLKDGGHYDAEVKTT |
| | | | YKAKKPVQLPGAYNVDIKLDILSHNEDYTIVEQYERAEGRHS |
| | | | TGGMDELYK |
| 64 | hApoB- | Protein | MDPPRPALLALLALPALLLLLLAGARAMVSKGEEDNMAIIKEF |
| | mCherry | | MRFKVHMEGSVNGHEFEIEGEGEGRPYEGTQTAKLKVTKG |
| | | | GPLPFAWDILSPQFMYGSKAYVKHPADIPDYLKLSFPEGFN |
| | | | WERVMNFEDGGVVTVTQDSSLQDGEFIYKVKLRGTNFPSD |
| | | | GPVMQCRTMGWEASTERMYPEDGALKGEIKQRLKLKDGG |
| | | | HYDAEVKTTYKAKKPVQLPGAYNVDIKLDILSHNEDYTIVEQ |
| | | | YERAEGRHSTGGMDELYK |
| 65 | gLuc- | Protein | MGVKVLFALICIAVAEAMVSKGEEDNMAIIKEFMRFKVHMEG |
| | mCherry | | SVNGHEFEIEGEGEGRPYEGTQTAKLKVTKGGPLPFAWDIL |
| | | | SPQFMYGSKAYVKHPADIPDYLKLSFPEGFNWERVMNFED |
| | | | GGVVTVTQDSSLQDGEFIYKVKLRGTNFPSDGPVMQCRTM |
| | | | GWEASTERMYPEDGALKGEIKQRLKLKDGGHYDAEVKTTY |
| | | | KAKKPVQLPGAYNVDIKLDILSHNEDYTIVEQYERAEGRHST |
| | | | GGMDELYK |
| 66 | hFVII- | Protein | MVSQALRLLCLLLGLQGCLAMVSKGEEDNMAIIKEFMRFKV |
| | mCherry | | HMEGSVNGHEFEIEGEGEGRPYEGTQTAKLKVTKGGPLPF |
| | | | AWDILSPQFMYGSKAYVKHPADIPDYLKLSFPEGFNWERVM |
| | | | NFEDGGVVTVTQDSSLQDGEFIYKVKLRGTNFPSDGPVMQ |
| | | | CRTMGWEASTERMYPEDGALKGEIKQRLKLKDGGHYDAEV |
| | | | KTTYKAKKPVQLPGAYNVDIKLDILSHNEDYTIVEQYERAEG |
| | | | RHSTGGMDELYK |

### REFERENCES

Hou, X., Zaks, T., Langer, R. et al. Lipid nanoparticles for mRNA delivery. Nature Reviews Materials 6, 1078-1094 (2021).
Cheng, Q., Wei, T., Farbiak, L., Johnson, L.T., Dilliard, S.A. & Siegwart, D.J. Selective organ targeting (SORT) nanoparticles for tissue-specific mRNA delivery and CRISPR-Cas gene editing. Nature Nanotechnology 15, 313-320 (2020).
Cheng, Q., Wei, T., Jia, Y., Farbiak, L., Zhou, K., Zhang, S., Wei, Y., Zhu, H. & Siegwart, D.J. Dendrimer-based lipid nanoparticles deliver therapeutic FAH mRNA to normalize liver function and extend survival in a mouse model of hepatorenal tyrosinemia type I. Advanced Materials 30, e1805308 (2018).
Liu, S.; Cheng, Q.; Wei, T.; Yu, X.; Johnson, L.T.; Farbiak, L. & Siegwart, D.J. Membrane-destabilizing ionizable phospholipids for organ-selective mRNA delivery and CRISPR-Cas gene editing. Nature Materials 20, 701-710 (2021).

What is claimed is:

1. An engineered signal peptide comprising an amino acid sequence of any one of SEQ. ID. NOS. 9-29 and variant or derivative thereof.

2. The engineered signal peptide of claim 1 fused to a heterologous polypeptide.

3. The engineered signal peptide of claim 2, wherein the heterologous polypeptide is a therapeutic or diagnostic polypeptide.

4. The engineered signal peptide of claim 3, wherein the heterologous polypeptide is any one of an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

5. The engineered signal peptide of claim 2, wherein the heterologous polypeptide is an enzyme, nutraceutical, food additive, taste enhancer, and/or cosmetic.

6. The engineered signal peptide of claim 2, wherein the heterologous polypeptide is a reporter polypeptide selected from a fluorescent protein, LacZ (b-galactosidase), CAT (chloramphenicol acetyltransferase), or luciferase.

7. The engineered signal peptide of claim 6, wherein the fluorescent protein is any one of GFP (green fluorescent protein), BFP (blue fluorescent protein), YFP (yellow fluorescent protein), RFP (red fluorescent protein), mCherry or luciferase.

8. A recombinant polynucleotide sequence comprising a nucleic acid sequence encoding the signal peptide of SEQ. ID. NOS. 9-29.

9. The recombinant polynucleotide sequence of claim 8, wherein the nucleic acid sequence is a DNA sequence.

10. The recombinant polynucleotide sequence of claim 8, wherein the nucleic acid sequence is an RNA sequence.

11. The recombinant polynucleotide sequence of claim 9, comprising a nucleic acid sequence of any one of SEQ. ID. NOS. 30-50 or variant or derivative thereof.

12. The recombinant polynucleotide sequence of claim 10, comprising a ribonucleic acid sequence corresponding to any one of SEQ. ID. NOS. 30-50 or variant or derivative thereof.

13. The recombinant polynucleotide of claim 8, further encoding a heterologous polypeptide in frame with the signal peptide.

14. The recombinant polynucleotide of claim 13, wherein the heterologous polypeptide is a therapeutic or diagnostic polypeptide.

15. The recombinant polynucleotide of claim 14, wherein the heterologous polypeptide is any one anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

16. The recombinant polynucleotide of claim 13, wherein the heterologous polypeptide is an enzyme, nutraceutical, food additive, taste enhancer, and/or cosmetic.

17. The recombinant polynucleotide of claim 13, wherein the heterologous polypeptide is a reporter polypeptide selected from a fluorescent protein, LacZ (b-galactosidase), CAT (chloramphenicol acetyltransferase), or luciferase.

18. The recombinant polynucleotide of claim 13, wherein the heterologous polypeptide is any one of SEQ ID. NOS. 55-58 or functional fragment, derivative or variant thereof.

19. The recombinant polynucleotide of claim 13, wherein the heterologous polypeptide is an anti-PD-L1 antibody, Enbrel, mCherry or hEPO or functional fragment, derivative or variant thereof.

20. A therapeutic composition comprising:
a. a delivery system; and
b. a polynucleotide sequence comprising a nucleic acid sequence encoding:
i. a signal peptide; and
ii. a therapeutic polypeptide.

21. The therapeutic composition of claim 20, wherein the signal peptide comprises an amino acid sequence of any one of SEQ. ID. NOS. 9-29 or variant or derivative thereof.

22. The therapeutic composition of claim 20, wherein the polynucleotide sequence encoding the signal peptide comprises a nucleic acid sequence corresponding to any one of SEQ. ID. NOS. 30-50 or variant or derivative thereof.

23. The therapeutic composition of claim 20, wherein the signal peptide is 10-50 amino acids in length

24. The therapeutic composition of any one of claims 20-23, wherein the delivery system is any one of a polymers, polyplexes, lipids, lipidoids, lipoplexes, liposomes, polymer nanoparticles, nanoparticles, lipid nanoparticles (LNPs), core-shell nanoparticles, solid lipid nanoparticles, metal nanoparticles, self-assembled nucleic acid nanoparticles, hyaluronidase, nanoparticle mimics, ribonucleoproteins, positively charged peptides, small molecule RNA-conjugates, aptamer-RNA chimeras, RNA-fusion protein complexes and any combination thereof.

25. The therapeutic composition of claim 24, wherein the delivery system is a lipid nanoparticle comprising an ionizable amino lipid.

26. The therapeutic composition of claim 25, wherein the lipid nanoparticle further comprises one or more of a phospholipid, cholesterol, or a polymer lipid.

27. The therapeutic composition of any one of claims 24-26, wherein the delivery system comprises any one of an iPhos LNPs, mDLNPs, liver SORT LNP, lung SORT LNP, or spleen SORT LNP.

28. The therapeutic composition of claim any one of claims 20-24, wherein the delivery system is a controlled system selected from a synthetic material depot, polymer depot, lipid depot, controlled release hydrogel depot, or controlled release polymer depot.

29. The therapeutic composition of any one of claims 20-28, further comprising one or more pharmaceutically acceptable excipients.

30. The therapeutic composition of claim 20, wherein the therapeutic polypeptide is any one of an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

31. A method of diagnosis, prophylaxis or treatment comprising, administration to a subject in need thereof, an effective amount of the compositions of any one of claims 20-30.

32. The method of diagnosis, prophylaxis or treatment of claim 31, wherein the administration is through one or more of a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or transdermal route.

33. The method of diagnosis, prophylaxis or treatment of claim 31, wherein the administration is via a controlled system selected from an implant, synthetic material depot, polymer depot, lipid depot, controlled release hydrogel depot, or controlled release polymer depot.

34. The method of diagnosis, prophylaxis or treatment of claim 31, wherein the subject is suspected of having or diagnosed with any one of an auto-immune disorder, cancer, diabetes, cardiovascular diseases, nerve disease, bacterial infection, fungal infection, viral infection, or fibrosis.

35. The method of diagnosis, prophylaxis or treatment of claim 31, wherein the subject is in need of prophylaxis.

36. The method of of diagnosis, prophylaxis or treatment of claim 31, wherein the therapeutic polypeptide of claim 15 is systemically secreted in the subject in need thereof.

37. The method of diagnosis, prophylaxis or treatment of claim 31, wherein the therapeutic polypeptide of claim 21 is directed for expression in any one of the lungs, liver or spleen.

38. The method of claim 31, wherein the subject is a mammal.

39. The method of claim 38, wherein the subject is a human.

40. Use of the compositions of any one of claims 20-30, for treatment of a subject in need thereof.

41. A recombinant polypeptide comprising:
i. a signal peptide corresponding to any one of SEQ ID NOS. 1-4 or variant or derivative thereof; and
ii. a heterologous polypeptide.

42. A recombinant polynucleotide comprising a nucleic acid sequence encoding:
iii. a signal peptide corresponding to any one of SEQ ID NOS. 1-4 or variant or derivative thereof; and
iv. a heterologous polypeptide in frame with the signal peptide.

43. The recombinant polynucleotide of claim 42, wherein the nucleic acid sequence is a DNA sequence.

44. The recombinant polynucleotide of claim 42, wherein the nucleic acid sequence is an RNA sequence.

45. The recombinant polynucleotide sequence of claim 43, comprising a nucleic acid sequence corresponding to any one of SEQ. ID. NOS. 5-8 or variant or derivative thereof.

46. The recombinant polynucleotide sequence of claim 44, comprising a ribonucleic acid sequence corresponding to SEQ. ID. NOS. 5-8 or variant or derivative thereof.

47. The recombinant polynucleotide of claim 42, wherein the heterologous polypeptide is a therapeutic or diagnostic polypeptide.

48. The recombinant polynucleotide of claim 47, wherein the heterologous polypeptide is any one anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

49. The recombinant polynucleotide of claim 42, wherein the heterologous polypeptide is an enzyme, nutraceutical, food additive, taste enhancer, and/or cosmetic.

50. The recombinant polynucleotide of claim 42, wherein the heterologous polypeptide is a reporter polypeptide selected from a fluorescent protein, LacZ (b-galactosidase), CAT (chloramphenicol acetyltransferase), or luciferase.

51. The recombinant polynucleotide of claim 42, wherein the heterologous polypeptide is an anti-PD-L1 antibody, Enbrel, hEPO or functional fragment, derivative or variant thereof.

52. A therapeutic composition comprising:
a. a delivery system; and
b. a polynucleotide sequence comprising a nucleic acid sequence encoding:
v. a signal peptide corresponding to any one of SEQ ID NOS. 1-4 or variant of derivative thereof; and
vi. a therapeutic polypeptide.

53. The therapeutic composition of claim 52, wherein the polynucleotide sequence encoding the signal peptide comprises a nucleic acid sequence of any one of SEQ. ID. NOS. 5-8 or variant or derivative thereof.

54. The therapeutic composition of claim 52, wherein the polynucleotide sequence encoding the signal peptide comprises a ribonucleic acid sequence corresponding to any one of SEQ. ID. NOS. 5-8 or variant or derivative thereof.

55. The therapeutic composition of any one of claims 52-54, wherein the delivery system is any one of a polymers, polyplexes, lipids, lipidoids, lipoplexes, liposomes, polymer nanoparticles, nanoparticles, lipid nanoparticles (LNPs), core-shell nanoparticles, solid lipid nanoparticles, metal nanoparticles, self-assembled nucleic acid nanoparticles, hyaluronidase, nanoparticle mimics, ribonucleoproteins, positively charged peptides, small molecule RNA-conjugates, aptamer-RNA chimeras, RNA-fusion protein complexes and any combination thereof.

56. The therapeutic composition of claim 55, wherein the delivery system is a lipid nanoparticle comprising an ionizable amino lipid.

57. The therapeutic composition of claim 56, wherein the lipid nanoparticle further comprises one or more of a phospholipid, cholesterol, or a polymer lipid.

58. The therapeutic composition of any one of claims 55-57, wherein the delivery system comprises any one of an iPhos LNPs, mDLNPs, liver SORT LNP, lung SORT LNP, or spleen SORT LNP.

59. The therapeutic composition of claim any one of claims 52-54, wherein the delivery system is a controlled system selected from a synthetic material depot, polymer depot, lipid depot, controlled release hydrogel depot, or controlled release polymer depot.

60. The therapeutic composition of any one of claims 52-59, further comprising one or more pharmaceutically acceptable excipients.

61. The therapeutic composition of claim 52, wherein the therapeutic polypeptide is any one of an anti-cancer, anti-inflammatory, immunomodulatory, anti-viral, anti-microbial, anti-fungal, anti-helminthic, hypocholestrolemic, anti-diabetic, anti-fibrotic, analgesics, anesthetics, anti-aging, anti-depressants, neuromodulatory, anti-dermatitis, anti-edema, anti-allergic, anti-hyperkeratolyte, antimycotic, anti-pruritic, cardiovascular therapeutic, chemotherapeutic, hormone, protein replacement therapeutic, polypeptide therapeutic, vaccine, viral vaccine, theragnostic, diagnostic or secreted therapeutic polypeptide.

62. A method of diagnosis, prophylaxis or treatment comprising, administration to a subject in need thereof, an effective amount of the compositions of any one of claims 52-61.

63. The method of diagnosis, prophylaxis or treatment of claim 62, wherein the administration is through one or more of a parenteral, oral, intraadiposal, intraarterial, intraarticular, intracranial, intradermal, intralesional, intramuscular, intranasal, intraocular, intrapericardial, intraperitoneal, intrapleural, intraprostatical, intrarectal, intrathecal, intratracheal, intratumoral, intraumbilical, intravaginal, intravenous, intravascular, intravitreal, liposomal, local, mucosal, parenteral, rectal, subconjunctival, subcutaneous, sublingual, topical, trans buccal, or transdermal route.

64. The method of diagnosis, prophylaxis or treatment of claim 63, wherein the administration is via a controlled system selected from an implant, synthetic material depot, polymer depot, lipid depot, controlled release hydrogel depot, or controlled release polymer depot.

65. The method of diagnosis, prophylaxis or treatment of claim 63, wherein the subject is suspected of having or diagnosed with any one of an auto-immune disorder, cancer, diabetes, cardiovascular diseases, nerve disease, bacterial infection, fungal infection, viral infection, or fibrosis.

66. The method of diagnosis, prophylaxis or treatment of claim 63, wherein the subject is in need of prophylaxis.

67. The method of treatment of claim 63, wherein the therapeutic polypeptide of claim 52 is systemically secreted in the subject in need thereof.

68. The method of diagnosis, prophylaxis or treatment of claim 63, wherein the therapeutic polypeptide of claim 52 is directed for expression in any one of the lungs, liver or spleen.

69. The method of claim 63, wherein the subject is a mammal.

70. The method of claim 63, wherein the subject is a human.

71. Use of the compositions of any one of claims 52-61 for treatment of a subject in need thereof.

72. The use as in claim 71, wherein the subject is suspected of or diagnosed with any one of an auto-immune disorder, cancer, diabetes, or fibrosis.

73. The use as in any one of claim 71 or 72, wherein the subject is a mammal.

74. The use as in any one of claim 71 or 72, wherein the subject is a human.
